(19) United States
(12) Patent Application Publication
(10) Pub. No.: US 2024/0050522 A1
(43) Pub. Date: Feb. 15, 2024

(54) COMPOSITIONS AND METHODS FOR PREVENTING, ATTENUATING, AND TREATING MEDICAL CONDITIONS WITH sHDL NANOPARTICLES

(71) Applicants: The Regents of the University of Michigan, Ann Arbor, MI (US); University of Kentucky Research Foundation, Lexington, KY (US)

(72) Inventors: Anna Schwendeman, Ann Arbor, MI (US); Hongliang He, Ann Arbor, MI (US); Sang Yeop Kim, Ann Arbor, MI (US); Xiang-An Li, Lexington, KY (US)

(21) Appl. No.: 18/266,542

(22) PCT Filed: Dec. 10, 2021
(86) PCT No.: PCT/US2021/062779

§ 371 Date: Jun. 9, 2023

Related U.S. Application Data
(60) Provisional application No. 63/124,403, filed on Dec. 11, 2020.

(51) Int. Cl. A61K 38/17; A61K 9/51; A61K 47/24; A61K 45/06; A61K 31/675; A61K 31/573; A61K 31/4706; A61P 31/00

(52) U.S. Cl. CPC A61K 38/1709; A61K 9/5123; A61K 47/24; A61K 45/06; A61K 31/675; A61K 31/573; A61K 31/4706; A61P 31/00

(57) ABSTRACT

Accordingly, the present invention relates compositions comprising synthetic HDL (sHDL) nanoparticles, methods for synthesizing such sHDL nanoparticles, as well as systems and methods utilizing such sHDL nanoparticles (e.g., in diagnostic and/or therapeutic settings). In particular, the present invention provides compositions comprising sHDL nanoparticles for purposes of preventing, attenuating, and/or treating sepsis and sepsis related disorders in a subject, conditions and symptoms caused by a viral infection (e.g., COVID-19)) in a subject, and conditions and symptoms caused by thrombosis in a subject.

### CROSS REFERENCE TO RELATED APPLICATIONS

The present invention claims the priority benefit of U.S. Provisional Patent Application 63/124,403, filed Dec. 11, 2020, which is incorporated by reference in its entirety.

### STATEMENT REGARDING FEDERALLY SPONSORED RESEARCH OR DEVELOPMENT

This invention was made with government support under GM 113832 awarded by the National Institutes of Health. The government has certain rights in the invention.

### FIELD OF THE INVENTION

The present invention relates compositions comprising synthetic HDL (sHDL) nanoparticles, methods for synthesizing such sHDL nanoparticles, as well as systems and methods utilizing such sHDL nanoparticles (e.g., in diagnostic and/or therapeutic settings). In particular, the present invention provides compositions comprising sHDL nanoparticles for purposes of preventing, attenuating, and/or treating sepsis and sepsis related disorders in a subject, conditions and symptoms caused by a viral infection (e.g., COVID-19)) in a subject, and conditions and symptoms caused by thrombosis in a subject.

### BACKGROUND OF THE INVENTION

Sepsis is a serious disease, and a major cause of death among patients hospitalized with serious illnesses, with a mortality rate of 30%. In spite of great advances in medical technology, sepsis is often caused by infections which occur following surgical operations, and this happens all around the world. In addition, bacterial infection in people with weak immune systems, such as infants and the elderly, may be especially liable to develop sepsis. For example, neonatal sepsis is known to affect 3 in 1,000 mature infants, with a 3- to 4-fold increase in attack rate for immature infants. Upon the onset of sepsis, the treatment thereof generally rests on antibiotics. If bacteria grow too excessively due to the absence of proper treatment, or if bacteria are highly resistant to antibiotics, the sepsis cannot be effectively treated with antibiotics alone.

As of July 2020, there are nearly 14 million total cases of COVID-19 reported worldwide causing nearly 600,000 deaths. The disease causes respiratory illness (like the flu) with symptoms such as a cough, fever, and in more severe cases, difficulty breathing. At present, the treatment is symptomatic, and oxygen therapy represents the major treatment intervention for patients with severe infection. Although several approaches have been proposed such as lopinavir/ritonavir (400/100 mg every 12 hours), hydroxychloroquine (200 mg every 12 hours), remdesivir, dexamethasone, and alpha-interferon (5 million units by aerosol inhalation twice per day), there is no specific antiviral treatment recommended for COVID-19, and no vaccine is currently available. Given the urgency of the COVID-19 outbreak, effective interventions against COVID-19 is a major challenge.

Under disease conditions such as atherosclerosis, excessive platelet reactivity often leads to the formation of arterial thrombi, the predominant cause of myocardial infarction and stroke. While current antiplatelet treatments inhibit platelet activation, their associated bleeding risk has greatly limited their successful clinical application.

Thus, there is a critical need to develop novel therapeutics for preventing, attenuating, and/or treating sepsis and sepsis related disorders in a subject, conditions and symptoms caused by a viral infection (e.g., COVID-19)) in a subject, and conditions and symptoms caused by thrombosis in a subject (e.g., a human subject).

The present invention addresses such needs.

### SUMMARY

HDL is the smallest and densest of the plasma lipoproteins consisting of apolipoprotein A-I (apoA-I) and phospholipids. HDL exerts a number of physiological functions, including cholesterol mobilization via reverse cholesterol transport. Additionally, HDL exerts an array of anti-inflammatory activities through a number of mechanisms. HDL preferentially binds to and neutralizes circulating endotoxin, returning it to the liver for elimination (6, 7). HDL also reduces TLR4 recruitment into lipid raft via cellular membrane cholesterol depletion (8). Furthermore, HDL induces activating transcription factor 3 (ATF3) expression to modulate TLR-induced pro-inflammatory cytokines (9-11).

Profound changes in the concentration and composition of HDL have been established in patients with sepsis (12-15). Multiple studies revealed that a marked decline in serum HDL levels was observed during infection and inflammation (12, 13, 15-17). Patients with sepsis have reductions in HDL-cholesterol (HDL-C) levels of 40-70% compared to healthy subjects and inflammation induced major changes in HDL composition (15, 18, 19). Further, a low level of HDL-C upon initiation of sepsis are associated with an increase in mortality and clinical outcomes (18).

The promising anti-inflammatory properties of HDL and inverse correlation between HDL-C level and mortality in sepsis fueled many nonclinical and clinical investigations assessing the role of HDL in sepsis via administration of reconstituted HDL (rHDL) (20). A majority of these studies utilized HDL protein or peptide, as the efficacy of naked apoA-I, apoA-I mutant, and apoA-I mimetic peptides have been reported (21-27). Nevertheless, the importance of HDL phospholipid composition has been largely neglected despite its role in intracellular signaling and receptor interactions (28). For instance, HDL sequesters LPS within its phospholipid layer to promote LPS neutralization and anti-inflammatory activity (29-31). Moreover, the protective mechanisms of HDL in sepsis remain poorly understood and treatment of sepsis via HDL infusion has yet to gamer significant attention despite the important relationship between HDL and sepsis.

Experiments conducted during the course of developing embodiments for the present invention investigated the impact of phospholipid on the anti-inflammatory activities of HDL. Several studies emphasized the importance of the physical state of phospholipid on HDL that can potentially impact the functionality in cholesterol efflux but not in anti-inflammatory activities. Notably, liquid crystalline unsaturated phospholipids promote more efficient cholesterol acceptor than gel phase saturated phospholipids due to more fluid liquid crystalline lipids are capable of greater exogenous lipid molecule (e.g. cholesterol) insertion in contrast to relatively rigid gel phase phospholipids (32). To this extent, it was hypothesized that HDL with a fluid liquid crystalline lipid phase would also result in enhanced anti-inflammatory activities by accelerating the efflux of exogenous molecules (e.g. LPS and cholesterol) and accessibility to phospholipid.

To investigate such a hypothesis, rHDLs were prepared by complexing apoA-I mimetic peptide, 22A, with different phosphatidylcholines (PC). 22A peptide was chosen as it retains the biological activity of endogenous apolipoprotein A-I and has shown favorable safety and pharmacokinetics in human clinical trials (33, 34). PC is the largest class of phospholipids, comprising 33-45% of total HDL lipid mass (35) and is recognized to contribute to the potent anti-inflammatory effect of HDL (28). Thus, PC was selected as the HDL phospholipid composition with variations in fatty acid chain length and saturation to produce different fluidity of PC phase on rHDL due to distinct phase transition temperature (Tₘ) of each phospholipid (1-palmitoyl-2-oleoyl-phosphatidylcholine, POPC; 1,2-dimyristoyl-sn-glycero-3-phosphatidylcholine, DMPC; 1,2-dipalmitoyl-sn-glycero-3-phosphatidylcholine, DPPC; and 1,2-distearoyl-sn-glycero-3-phosphatidylcholine, DSPC) and examined the impact of fluidity of rHDL on its anti-inflammatory activities against LPS-induced inflammation in vitro and in vivo.

Such experiments demonstrated that changes in phospholipid composition of rHDL significantly enhance the anti-inflammatory activities due to different fluidity of rHDL. Fluid liquid crystalline phase rHDLs, particularly 22A-DMPC, most effectively modulated NF-κB signaling and pro-inflammatory mediators via TLR4 signaling as compared to rigid gel phase rHDL. Interestingly, only 22A-DMPC further reduced TLR4 recruitment into lipid rafts and promoted ATF3 expression which significantly modulated inflammatory activity with rHDL pre-treatment. Accordingly, 22A-DMPC improved mortality and protected organs from inflammatory injury in mice challenged with a lethal dose of LPS.

Experiments conducted during the course of developing embodiments for the present invention utilized a new generation of synthetic HDL (sHDL), made up of a 22 amino acid ApoA1 mimetic peptide (22A) and phospholipids sphingomyelin (SM) and 1,2-dipalmitoyl-sn-glycero-3-phosphocholine (DPPC) (22A/SM-DPPC). The binding of phospholipids to mimetic peptides leads to formation of small nano-discs—sHDL. The interaction between peptides and phospholipids favorably increases the stability of the particles over naked peptides, such as 4F, thereby increasing its circulation half-life from 1-2 hours (naked peptide) to >12 hours (see, M. Khan, N. et al., Circulation, vol. 108, pp. 563-564; J. Miles, et al., Arteriosclerosis Thrombosis and Vascular Biology, vol. 24, pp. E19-E19; B. A. Di Bartolo, et al., Atherosclerosis 217, 395-400 (2011)). Additionally, such a sHDL is composed entirely of synthetic materials, eliminating the caveats associated with using plasma-purified protein.

The pathogenic mechanism of SARS-CoV-2 infection and COVID-19 disease leading to pneumonia and ADRS seems to be particularly complex. The virus binds to the angiotensin-converting enzyme 2 (ACE2) receptor in humans, expressed in the endothelium, kidney, lung and heart. Viral infection leads to “cytokine storm”, the deadly uncontrolled systemic inflammatory response resulting from immune effector cell-medicated release of large quantity of pro-inflammatory cytokines (TNF-α, IFN-γ, IL-1β, IL-6). The proinflammatory cells results in endothelial cell activation, endothelial leakage and multiple organ failure. At the same time, platelet and fibrinogen are activated, leading to formation of multiple blood clots. Blood clots accumulate and form venous thrombosis, which is the main cause of septic shock symptoms such as diffuse intravascular coagulation (DIC). The venous thrombi flow to the lung and block pulmonary blood vessels, resulting in low oxygen blood oxygen, hypoxia, and sudden death. FIG. 15 schematically represents dynamics of COVID-19 pathology and a potential protective mechanism of sHDL infusions in COVID-19 through: 1) modulation of lipid raft composition resulting in reduced levels ACE2 and SARS-COV-2 virus cell entry; 2) inhibition of SARS-COV2 S protein induced NF-kB activation and reduction of proinflammatory cytokine release by immune effector cells; 3) inhibiting endothelial activation and dysfunction; 4) inhibition of platelet aggregation and thrombus formation.

Abundant clinical evidence has established an inverse correlation between high-density lipoprotein (HDL) cholesterol levels and the risk of thrombosis. Those clinical data suggest that raising HDL levels may be an important therapeutic strategy to reduce platelet hyperreactivity and the risk of thrombosis. In addition to its protective role in vascular endothelium, native HDL is reported to promote cholesterol efflux from platelets and change lipid raft organization in the platelet membrane, thus preventing platelet hyperreactivity. By mimicking the composition of native HDL, the present invention provides synthetic HDL (sHDL) infusion for reduction of platelet hyperactivity and prevention of thrombus formation. Indeed, experiments conducted during the course of developing embodiments for the present invention shows that sHDL, consisting of an apolipoprotein mimetic peptide and 1,2-dimyristoyl-sn-glycero-3-phosphocholine, significantly reduced platelet aggregation both in vitro and ex vivo as well as thrombus formation in vivo. Therefore, sHDL infusion provides a safer and more effective antithrombotic strategy to address the current clinical complications of antiplatelet agents by modifying the sHDL composition, understanding the potential mechanisms by which sHDL regulates platelet activity, and investigating the comprehensive in vivo performance of sHDL.

Accordingly, the present invention relates compositions comprising synthetic HDL (sHDL) nanoparticles, methods for synthesizing such sHDL nanoparticles, as well as systems and methods utilizing such sHDL nanoparticles (e.g., in diagnostic and/or therapeutic settings). In particular, the present invention provides compositions comprising sHDL nanoparticles for purposes of preventing, attenuating, and/or treating sepsis and sepsis related disorders in a subject, conditions and symptoms caused by a viral infection (e.g., COVID-19)) in a subject, and conditions and symptoms caused by thrombosis in a subject.

In certain embodiments, the present invention provides compositions comprising a synthetic HDL nanoparticle (sHDL) for preventing, attenuating, and/or treating sepsis and sepsis related disorders in a subject, conditions and symptoms caused by a viral infection (e.g., COVID-19)) in a subject, and conditions and symptoms caused by thrombosis in a subject, wherein the sHDL comprises a mixture of at least one HDL apolipoprotein and at least one lipid component.

In certain embodiments, the present invention provides methods of preventing, attenuating or treating a subject having or at risk for having sepsis (e.g., LPS induced sepsis) or a sepsis related disorder, comprising administering to the subject a composition comprising a sHDL, wherein the sHDL comprises a mixture of at least one HDL apolipoprotein and at least one lipid component. In some embodiments, administration of the composition results in attenuation of inflammatory activity in the subject through, for example, suppression of NF-kB signaling, regulating TLR4 recruitment into lipid rafts, promoting ATF-3 expression, protecting organs from organ failure, and neutralization of LPS. In some embodiments, the sHDL nanoparticle is made up of 22A and SM-DMPC.

In some embodiments, the sepsis related disorder is any condition associated with bacteremia or introduction of lipopolysaccharide into the blood stream or onto an extra-gastrointestinal mucosal surface. In some embodiments, the sepsis related disorder is a condition selected from endotoxin-related shock, endotoxin-related disseminated intravascular coagulation, endotoxin-related anemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leucopenia resulting from Gram-negative infection, hemodynamic shock and endotoxin-related pyresis.

In some embodiments for preventing, attenuating or treating a subject having or at risk for having sepsis (e.g., LPS induced sepsis) or a sepsis related disorder, the composition comprising a sHDL is co-administered with one or more of the following therapeutic agents: alpha-/beta-adrenergic agonists (e.g., norepinephrine, dopamine, dobutamine, epinephrine, vasopressin, phenylephrine), isotonic crystalloids, albumin, antibiotics (e.g., cefotaxime, ticarcillin-clavulanate, piperacillin-tazobactam, imipenem-cilastatin, meropenem, clindamycin, metronidazole, ceftriaxone, ciprofloxacin, cefepime, levofloxacin, vancomycin), and corticosteroids (e.g., hydrocortisone, dexamethasone).

In certain embodiments, the present invention provides methods of preventing, attenuating or treating a subject having or at risk for having conditions and symptoms caused by a viral infection (e.g., COVID-19)), comprising administering to the subject a composition comprising a sHDL, wherein the sHDL comprises a mixture of at least one HDL apolipoprotein and at least one lipid component. In some embodiments, administration of the composition results in, for example, modulation of lipid raft composition resulting in reduced levels ACE2 and SARS-COV-2 virus cell entry; inhibition of SARS-COV2 S protein induced NF-kB activation and reduction of proinflammatory cytokine release by immune effector cells; and inhibiting endothelial activation and dysfunction.

In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having M^{pro} protease activity and/or expression.

In some embodiments, the one or more symptoms related to viral infection includes, but is not limited to, fever, fatigue, dry cough, myalgias, dyspnea, acute respiratory distress syndrome, and pneumonia.

In some embodiments, the present invention provides methods for treating, ameliorating and/or preventing acute respiratory distress syndrome and/or pneumonia in a subject, comprising administering to the subject a composition comprising a sHDL, wherein the sHDL comprises a mixture of at least one HDL apolipoprotein and at least one lipid component. In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments for preventing, attenuating or treating a subject having or at risk for having conditions and symptoms caused by a viral infection (e.g., COVID-19), the composition comprising a sHDL is co-administered with one or more of the following therapeutic agents: remdesivir, dexamethasone, and hydroxychloroquine.

In certain embodiments, the present invention provides methods of preventing, attenuating or treating a subject having or at risk for having conditions and symptoms caused by thrombosis, comprising administering to the subject a composition comprising a sHDL, wherein the sHDL comprises a mixture of at least one HDL apolipoprotein and at least one lipid component. In some embodiments, administration of the composition results in, for example, reduction of platelet activity, prevention of thrombus formation, and reduction of platelet aggregation.

In some embodiments, the conditions and symptoms caused by thrombosis are related to a venous thrombosis. In some embodiments, the conditions and symptoms caused by thrombosis are related to an arterial thrombosis.

In some embodiments, thrombosis is a feature of an underlying disease or condition. Non-limiting examples of such disease or condition include acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, thromboembolic stroke, systemic embolism, ischemic stroke, venous thromboembolism, atrial fibrillation, non-valvular atrial fibrillation, atrial flutter, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the conditions and symptoms caused by thrombosis are selected from the group consisting of embolic stroke, thrombotic stroke, venous thrombosis, deep venous thrombosis, acute coronary syndrome, and myocardial infarction.

In some embodiments for preventing, attenuating or treating a subject having or at risk for having conditions and symptoms caused by thrombosis, the composition comprising a sHDL is co-administered with one or more of the following therapeutic agents: heparin; tPA; anistreplase; streptokinase; urokinase; a coumadin; warfarin; idraparinux; fondaparinux; aspririn; an adenosine diphosphate receptor inhibitor; a phosphodiesterase inhibitor; a glycoprotein IIB/IIA inhibitor; an adenosine reuptake inhibitor; and a thromboxane receptor antagonist.

In such embodiments for preventing, attenuating, and/or treating sepsis and sepsis related disorders in a subject, conditions and symptoms caused by a viral infection (e.g., COVID-19)) in a subject, and conditions and symptoms caused by thrombosis in a subject, the administering to the subject a therapeutically effective amount of a composition comprising a sHDL comprises a continuous infusion of sHDL and/or non-continuous infusions of sHDL.

In some embodiments, the subject is a human being.

In such embodiments, the sHDL is not limited to a particular size. In some embodiments, the average particle size of the sHDL nanoparticle is at or between 6-20 nm. In some embodiments, the average particle size of the sHDL nanoparticle is at or between 7-12 nm.

In some embodiments, the sHDL comprises a mixture of at least one HDL apolipoprotein component and at least one lipid component.

In some embodiments, the molar ratio of the HDL apolipoprotein component to the lipid component is about 2:1 to 200:1.

In some embodiments, the lipid component comprises a combination of one or any combination of sphingomyelin (SM), D-erythrose-sphingomyelin, D-erythrose dihydrosphingomyelin, palmitoylsphingomyelin, lysophospholipids, galactocerebroside, gangliosides, cerebrosides, glycerides, triglycerides, diglycerides, small alkyl chain phospholipids, phosphatidylcholine, egg phosphatidylcholine, soybean phosphatidylcholine, dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), 1,2-distearoyl-sn-glycero-3-phosphatidylcholine (DSPC), distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, ceramides, a phosphatidylserine, dimyristoylphosphatidylserine, dipalmitoylphosphatidylserine, brain phosphatidylserine, brain sphingomyelin, egg sphingomyelin, milk sphingomyelin, palmitoyl sphingomyelin, phytosphingomyelin, dipalmitoylsphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives, lyso-phosphotydyl choline, lyso-sphingomyelin, dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], Lyso phoshphatidic acid, Lyso phosphatidylcholine, OA-NO₂ (nitrated oleic acid 9- and 10-nitro-cis-octedecenolic acids), LNO₂ (nitrated linoleic Acid 9-, 10-, 12- and 13-nitro-cis-octedecadienoic acids), AA-NO₂ (nitrated Arachidonic Acid 5-, 6-, 8-, 9-, 11-, 12-, 14,- and 15-nitro-cis-eicosatetraenoic acids), CLNO₂ (nitrated cholesteryl linoleate cholestaryl-9-, 10-, 12- and 13-nitro-cis-octedecadiencates), fatty acid, omega-3 polyunsaturated fatty acids, hexadecatrienoic acid (HTA; 16:3 (n-3); all-cis-7,10,13-hexadecatrienoic acid), a-Linolenic acid (ALA; 18:3 (n-3); all-cis-9,12,15-octadecatrienoic acid), stearidonic acid (SDA; 18:4 (n-3); all-cis-6,9,12,15-octadecatetraenoic acid), eicosatrienoic acid (ETE; 20:3 (n-3); all-cis-11,14,17-eicosatrienoic acid), eicosatetraenoic acid (ETA; 20:4 (n-3); all-cis-8,11,14,17-eicosatetraenoic acid), eicosapentaenoic acid (EPA; 20:5 (n-3); all-cis-5,8,11,14,17-eicosapentaenoic acid), heneicosapentaenoic acid (HPA; 21:5 (n-3); all-cis-6,9,12,15,18-heneicosapentaenoic acid); docosapentaenoic acid (DPA; clupanodonic acid; 22:5 (n-3); all-cis-7,10,13,16,19-docosapentaenoic acid), docosahexaenoic acid (DHA; 22:6 (n-3); all-cis-4,7,10,13,16,19-docosahexaenoic acid), tetracosapentaenoic acid; 24:5 (n-3); all-cis-9,12,15,18,21-tetracosapentaenoic acid), tetracosahexaenoic acid (Nisinic acid; 24:6 (n-3), all-cis-6,9,12,15,18,21-tetracosahexaenoic acid), sphingosine-1-phosphate analogs, sphingosine-1-phosphate antagonists, sphingosine-1-phosphate agonists, sphingosine-1-phosphate receptor agonists, sphingosine-1-phosphate receptor antagonists, and sphingosine-1-phosphate receptor analogs.

In some embodiments, the lipid component comprises neutral phospholipids, negatively charged phospholipids, positively charged phospholipids, or a combination thereof. In some embodiments, the fatty acid chains on the phospholipids are preferably from 12 to 26 or 16 to 26 carbons in length and can vary in degree of saturation from saturated to mono-unsaturated.

In some embodiments, the HDL apolipoprotein component is selected from the group consisting of apolipoprotein A-I (apo A-I), apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

In some embodiments, the ApoA-I mimetic is described by any of SEQ ID NOs: 1-336 and WDRVKDLATVYVDVLKDSGRDYVSQF (SEQ ID NO: 337), LKLLDNWDSVTSTFSKLREOL (SEQ ID NO: 338), PVTOEFWDNLEKETEGLROEMS (SEQ ID NO: 339), KDLEEVKAKVQ (SEQ ID NO: 340), KDLEEVKAKVO (SEQ ID NO: 341), PYLDDFQKKWQEEMELYRQKVE (SEQ ID NO: 342), PLRAELQEGARQKLHELOEKLS (SEQ ID NO: 343), PLGEEMRDRARAHVDALRTHLA (SEQ ID NO: 344), PYSDELRQRLAARLEALKENGG (SEQ ID NO: 345), ARLAEYHAKATEHLSTLSEKAK (SEQ ID NO: 346), PALEDLROGLL (SEQ ID NO: 347), PVLESFKVSFLSALEEYTKKLN (SEQ ID NO: 348), PVLESFVSFLSALEEYTKKLN (SEQ ID NO: 349), PVLESFKVSFLSALEEYTKKLN (SEQ ID NO: 350), TVLLLTICSLEGALVRRQAKEPCV (SEQ ID NO: 351), QTVTDYGKDLME (SEQ ID NO: 352), KVKSPELOAEAKSYFEKSKE (SEQ ID NO: 353), VLTLALVAVAGARAEVSADOVATV (SEQ ID NO: 354), NNAKEAVEHLOKSELTOOLNAL (SEQ ID NO: 355), LPVLVWLSIVLEGPAPAOGTPDVSS (SEQ ID NO: 356), LPVLVVVLSIVLEGPAPAQGTPDVSS (SEQ ID NO: 357), ALDKLKEFGNTLEDKARELIS (SEQ ID NO: 358), VVALLALLASARASEAEDASLL (SEQ ID NO: 359), HLRKLRKRLLRDADDLQKRLAVYOA (SEQ ID NO: 360), AQAWGERLRARMEEMGSRTRDR (SEQ ID NO: 361), LDEVKEQVAEVRAKLEEQAQ (SEQ ID NO: 362), DWLKAFYDKVAEKLKEAF (SEQ ID NO: 363), DWLKAFYDKVAEKLKEAFPDWAKAAYDKAAEKAKEAA (SEQ ID NO: 364), PVLDLFRELLNELLEALKQKL (SEQ ID NO: 365), PVLDLFRELLNELLEALKQKLA (SEQ ID NO: 366), PVLDLFRELLNELLEALKQKLK (SEQ ID NO: 367), PVLDLFRELLNELLEALKQKLA (SEQ ID NO: 368), PVLDLFRELLNELLEALKKLLK (SEQ ID NO: 369), PVLDLFRELLNELLEALKKLLA (SEQ ID NO: 370), and PLLDLFRELLNELLEALKKLLA (SEQ ID NO: 371).

In some embodiments, the ratio of HDL apolipoprotein component to lipid component is at or between 1:1 to 1:4 wt/wt. In some embodiments, the ratio of HDL apolipoprotein component to lipid component is at or between 1:1.5 to 1:3 wt/wt. In some embodiments, the ratio of HDL apolipoprotein component to lipid component is 1:2 wt/wt.

In some embodiments, the sHDL nanoparticle has less than 5% free lipid component impurity. In some embodiments, the sHDL nanoparticle has less than 20% free HDL apolipoprotein component impurity.

In some embodiments, approximately 25% of the lipid component is cholesterol and/or cholesterol ester. In some embodiments, approximately 10% of the lipid component is cholesterol and/or cholesterol ester. In some embodiments, approximately 5% of the lipid component is cholesterol and/or cholesterol ester. In some embodiments, approximately 1% of the lipid component is cholesterol and/or cholesterol ester.

In some embodiments, the composition comprising sHDL is at least 90%, at least 92.5%, at least 95%, at least 96%, at least 97% or at least 98% pure.

In some embodiments, the composition comprising sHDL is at least 80%, at least 85%, at least 90% or at least 95% homogeneous, as reflected by a single peak in gel permeation chromatography.

In some embodiments, at least 80%, at least 85%, at least 90% or at least 95% of the sHDL nanoparticles range 4 nm to 12 nm in size, 6 nm to 12 nm in size, or 8 nm to 12 nm in size, as measured by GPC or DLS.

In some embodiments, at least 95%, at least 96%, at least 97%, at least 98% or at least 99% of the HDL apolipoprotein component is in complexes.

Additional embodiments will be apparent to persons skilled in the relevant art based on the teachings contained herein.

### BRIEF DESCRIPTION OF THE DRAWINGS

FIG. 1. Characterization of rHDL. A: Transmission electron microscopy image of different sHDL. B: Size distribution profile of different rHDL analyzed by dynamic light scattering. C: Size distribution and purity profile of different rHDL via gel permeation chromatography.

FIG. 2. Cell viability with rHDL treatment. A-C: Viability of Raw 264.7 (A), J774A.1 (B), HEK-Blue hTLR4 (C) cells with incubation of different formulations and concentrations rHDL for 18 h.

FIG. 3. Absorbance profiles of fluorescent-LPS bound to rHDL. Fluorescent-LPS (10 μg/mL) and different formulations of rHDL (1 mg/mL) were mixed and incubated for 1 hour at 37° C. and the rHDL-LPS mixture was analyzed by HPLC with fluorescence detector. Blue dashed area represents the area which rHDL present in UV absorbance.

FIG. 4. Modulation of inflammatory response with different rHDL treatment A: Activation of NF-kB of HEK-BLUE hTLR4 cells with treatment of different formulations and concentrations of rHDL in a presence of LPS (2 ng/mL). B-D: Concentration of TNF-α (B), IL-6 (C), and MCP-1 (D) of macrophages with treatment of different formulations and concentrations of rHDL in a presence of LPS (2 ng/mL). *P<0.05, **P<0.01, ***P<0.001 compared with LPS group; #P<0.05 compared with 22A-DSPC group.

FIG. 5. Disruption of lipid raft content and TLR-4 with rHDL. A: Relative quantification of cholesterol efflux from radio-labeled cholesterol-loaded macrophages with different formulations of rHDL (100 μg/mL) to the non-treated control group. Percentage cholesterol efflux from radio-labeled cholesterol-loaded macrophages was reported by liquid scintillation counting (n=4, mean f SEM). B-C: Relative measurement of lipid raft content of macrophages (B) and TLR4 expression (C) with different formulations of rHDL (100 μg/mL) to the non-treated control group. Percentage of lipid raft content and TLR4 expression was reported by the mean fluorescence intensity from flow cytometry. ns: not significant, *P<0.05, **P<0.01, ***P<0.001 compared with LPS group; #P<0.05 compared with 22A-DSPC group.

FIG. 6. The expression of ATF3 mRNA and protein with rHDL. A: Kinetics of ATF3 mRNA expression in macrophages with different formulations of rHDL (100 μg/mL) (n=9 f SEM). B: ATF3 protein expression in macrophages by immunoblot with different formulations of rHDL (100 μg/mL) followed by 18 h incubation. ***P<0.001.

FIG. 7. Attenuation of inflammatory response with pretreatment of rHDL. A: Activation of NF-kB of HEK-BLUE hTLR4 cells. Cells were stimulated with 2 ng/mL of LPS after washing out the 18 h pre-treatment of different formulations and concentrations. B-D: Concentration of TNF-α (B), IL-6 (C), and MCP-1 (D) of macrophages. Macrophages were initially incubated with different formulations and concentrations of rHDL for 18 h, then rHDL were completely removed and stimulated with LPS (2 ng/mL). *P<0.05, **P<0.01, ***P<0.001.

FIG. 8. Serum cytokine levels post LPS-only or LPS-rHDL mixture administration. A-C: The LPS was incubated in the absence or presence of different formulations of rHDL for 30 min at 37° C. Experimental groups were with LPS-rHDL mixtures at 10 mg/kg of rHDL and 0.05 mg/kg of LPS. Blood was collected at 2 h post-administration and the concentrations of TNF-α (A), IL-6 (B), and MCP-1 (C) were measured (n=5). ns: not significant, ***P<0.001.

FIG. 9. Serum cytokine levels post rHDL administration from endotoxemia model. A-C: Different formulation of rHDL was administered via intravenous injection at 10 mg/kg. Subsequently, LPS was challenged via intraperitoneal injection at 0.05 mg/kg. Blood was collected at 2 h post-administration and the levels of TNF-α (A), IL-6 (B), and MCP-1 (C) were measured (n=10). ns: not significant, *P<0.05, **P<0.01, ***P<0.001.

FIG. 10. The effect of 22A-DMPC treatment on LPS-induced lethality in mice. Mice were first challenged with LPS at 10 mg/kg (i.p.). After the anal temperature had risen 0.5° C. (approximately 15 min), mice were administered PBS or 22A-DMPC at 10 mg/kg (i.v.). Survival was then monitored every 6 h for 96 h. Data show survival proportions (%) (n=10). **P<0.01.

FIG. 11. Effect of treatment with 22A-sHDL on LPS-induced lethality in mice 22A-DMPC Survival rate (%) of mice challenged with LPS of different doses. Mice were injected with 5, 10, or 20 mg/kg of LPS (i.p.) without any treatment with 22A-sHDL (A). Mice were divided into control, LPS (10 mg/kg) only and LPS (10 mg/kg)+22A-DMPC (10 mg/kg) treatment groups. After the anal temperature had risen 0.5° c., mice were either treated with saline or 22A-DMPC (B). Survival was then monitored every 6 hr for 4 days. Data show survival proportions (%) (n=10). **P<0.01.

FIG. 12. Representative histologic images of the liver. A: Negative control group with normal levels of glycogen storage (evident as irregularly vacuolated hepatocytes) and no inflammatory infiltration. B: 22A-DMPC treatment group with the same appearance as control. C: LPS group with diffuse glycogen depletion (no hepatocyte vacuolation) and inflammatory cell infiltration within sinusoids and central venules (arrows). Original magnification 400×. Bars 20 μm.

FIG. 13. Representative histologic images of the lung at low magnification overview (large image) and at higher magnification (insets). A: Normal lung in control showing open alveolar spaces separated by thin, delicate alveolar septae without interstitial expansion. B: Normal lung in the 22A-DMPC treatment group. C: Low level inflammatory cell infiltration evidenced as neutrophil emigration from pulmonary venules (arrowheads) and multifocal expansion of the interstitium by neutrophils and macrophages (arrows) (C). Original magnifications 100× (large image) and 400× (insets). Bars 100 μm (large image) and 20 μm (insets).

FIG. 14. The effect of 22A-DSPC treatment on LPS-induced lethality in mice. Mice were challenged with LPS via at 10 mg/kg (i.p.). After the anal temperature had risen 0.5° C. (approximately 15 min), mice were administered in the absence or presence of 22A-DSPC at 10 mg/kg (i.v.). Survival was then monitored every 6 h for 96 h. Data show survival proportions (%) (n=10). **P<0.01.

FIG. 15. FIG. 15 schematically represents dynamics of COVID-19 pathology and a potential protective mechanism of sHDL infusions in COVID-19 through: 1) modulation of lipid raft composition resulting in reduced levels ACE2 and SARS-COV-2 virus cell entry; 2) inhibition of SARS-COV2 S protein induced NF-kB activation and reduction of proinflammatory cytokine release by immune effector cells; 3) inhibiting endothelial activation and dysfunction; 4) inhibition of platelet aggregation and thrombus formation.

FIG. 16. Serum total cholesterol and HDL cholesterol levels in healthy controls (n=71, 38M, 33F) and COVID-19 patients (n=80, 42M, 38F), ***p<0.001. B. The level of HDL-C in a critically ill COVID-19 patient dropped persistently until the day 9, and recovered slowly until hospital discharge as patient recovered (Xu et al, Lancet, 2020) C Septic patients have a marked decrease in HDL-cholesterol levels on the day of admission to the Univ. of Michigan ICU in patients without sepsis (black, n=75), with sepsis that later survived (blue, n=181) and with sepsis that later expired (red, n=35) (mean f SEM). (D) HDL-C levels over 14 days in septic patients with HDL-C<10 mg/dL at day 0. Statistical significance determined by one-way ANOVA with Tukey's multiple comparison test.

FIG. 17. ETC-642 treatment restores HDL levels, and protects CLP-induced animal death and vascular leakage. B6 mice were subjected to CLP (21G needle, ⅔ ligation). 2h post CLP, the mice were treated with/without 7.5 mg/kg ETC-642 (i.v.). A) HDL-cholesterol concentrations. Plasma was collected 18h post CLP. B) 7d Animal survival (PBS n=21; ETC-642 n=19). C) Body temperature (Sham, n=4; PBS and ETC-642, n=8) D) Plasma IL-6 concentration at 18h post CLP (Sham, n=4; PBS and ETC-642, n=8). E) Evans Blue assay of lung endothelium leakage 24h post CLP. Evans Blue was injected 45 min prior to sacrificing the mice. After perfusion with PBS/EDTA for 20 min, the lungs were excised, weighed, extracted in formamide and measured at A610/740 nm (Sham, n=3; PBS, n=4; sHDL, n=6). Mean±SEM.

FIG. 18. A) Dual recognition of SARS-CoV-2 to ACE-2 receptor and lipid rafts on cell membranes (Fantini, Int J Antimicrob Agents, 2020). B) Depleting lipid rafts reduced the infectivity of SARS-CoV pseudovirus (S) (Lu, Biochem. Biophys. Res. Commun., 2008). C) sHDLs disrupt lipid rafts on cell membranes. D) sHDL treatment reduced TLR-4 in lipid raft (LR) fractions on RAW 264.7 macrophages. (PNS: post-nuclear supernatant; CYT: cytosol; IM: intracellular membrane; PM: plasma membrane.)

FIG. 19. sHDL inhibits platelet aggregation in vitro. Washed human platelets (3×10⁸ platelets/mL) were re-incubated with sHDL for 30 min followed by activation by 0.25 nmol/L thrombin (A) or 1 μg/mL collagen (B); * p<0.05; ** p<0.01 and *** p<0.001. Mice were dosed with sHDL (50 mg/kg) or saline, thrombus (30-50 μm diameter) were induced in the arterioles by a laser ablation system. Images of thrombus formation (C) at the site of injured arterioles were acquired in real-time under 63× water-immersion objective with a Zeiss Axio Examiner Z1 fluorescent microscope equipped with solid laser launch system and high-speed sCMOS camera. Fluorescent intensity over the time course of thrombus formation were analyzed (D).

FIG. 20. Platelets effectively uptake sHDL. A, Isolated human platelets were incubated with DiO-sHDL (50 μg/mL 22A peptide, 2.5 μg/mL DiO) for 30 minutes, and the uptake of DiO-sHDL by human platelets was monitored by fluorescent microscopy. B-C, Platelets were stained by CD41-PE antibody, and whole blood flow cytometry for DiO signal in platelets after 30 minutes of DiO-sHDL injection to mice.

FIG. 21. Phospholipid composition impacts the inhibitory effect of sHDL on platelet activation and aggregation. A-B, Isolated human platelets were incubated with various sHDL (200 μg/mL 22A peptide) for 30 minutes, and platelets were subject to different agonist-induced platelet aggregations. C-D, Whole blood were pretreated with various sHDL (200 μg/mL 22A peptide) for 30 minutes and analyzed for blood coagulation.

FIG. 22. sHDL dose-dependently inhibits platelet activation and aggregation. A-B, Isolated human platelets were incubated with various sHDL (0.05, 0.1, 0.2 and 0.4 mg/mL 22A peptide) for 30 minutes, and platelets were subject to different agonist-induced platelet aggregations. C-D, sHDL dose-dependently attenuates platelet adhesion, aggregation, and thrombus formation under arterial flow conditions. Heparin-anticoagulated whole blood was incubated with sHDL (0.1, 0.2 and 0.4 mg/mL) for 30 minutes followed by perfusion at arterial shear over a collagen-coated surface.

FIG. 23. sHDL naturally homes to newly formed thrombus and effectively inhibits thrombus formation in a laser-induced cremaster arteriole thrombosis model. A, Male mice were pretreated with DiO-sHDL IV at 50 mg/kg of 22A peptide, 2.5 mg/kg of DiO. After 24 hours, Alexa Flour 647 rat-anti mouse CD62P (3 μg) was administered by a jugular vein cannula prior to vascular injury. Multiple independent thrombi were induced in the arterioles (30-50 μm diameter) of each mouse by a laser ablation system. Images of thrombus formation at the site of injured arterioles were acquired in real-time under 63× water-immersion objective with a Zeiss Axio Examiner Z1 fluorescent microscope. Mice were dosed with sHDL (50 mg/kg) or saline, and thrombi (30-50 μm diameter) were induced in the arterioles by a laser ablation system. Images of thrombus formation (B) at the site of injured arterioles and fluorescent intensity over the time course of thrombus formation were analyzed (C).

FIG. 24A-F: Septic patients have lower HDL levels, which is associated with poor survival. At time of intake (Day 0), there is a significant difference between plasma concentrations of HDL-C(A) and ApoA-1 (B) in expired sepsis patients versus non-sepsis controls. Patients entering the ICU with HDL-C levels <10 mg/dL had a higher incidence of death than those with HDL-C levels >10 mg/dL (C); those with ApoA-1 levels <30 mg/mL also had a higher incidence of death than those with ApoA-1>30 mg/mL (D); HDL-C(E) and ApoA-1 (F) levels over 14 days in septic patients with HDL-C<10 mg/dL at day 0; statistical significance determined by one-way ANOVA with Tukey's multiple comparison test.

FIG. 25A-D: sHDL preparation and characterization. Schematic of sHDL preparation procedure (A); Transmission electron microscopy (TEM) image shows nanosized, discoidal sHDL particles (B); gel permeation chromatography (GPC) profile of sHDL indicates >99% purity (C); Size distribution of sHDL particles is 10.43±3.283 nm with a PDI of 0.112 as determined by dynamic light scattering (DLS) (D).

FIG. 26A-F: sHDL inhibits LPS/TNF-α-induced endothelial cell activation and inflammatory cytokine production. Human umbilical vein endothelial cells (HUVEC) were treated with LPS in the presence of sHDL at the concentrations indicated in the figure. Fold change in gene expression of VCAM-1 (A), ICAM-1 (B), E-selectin (C), and eNOS (D) were determined by RT-qPCR and normalizing to endogenous GAPDH expression. Concentrations of cytokines IL-6 (E) and IL-8 (F) in the supernatant were measured by ELISA.

FIG. 27: sHDL particle retention time analyzed by gel permeation chromatography (GPC). sHDL complexes elutes at approximately 7 min and profile of sHDL indicates >98% purity.

FIG. 28: sHDL particle size distribution analyzed by dynamic light scattering (DLS). sHDL complexes displays diameters ranging from 8-10 nm illustrating that diameters of sHDL complexes have size distributions equivalent to that of native HDL.

FIG. 29A-I: sHDL complexes inhibit LPS-induced NF-κB activation in a lipid component and concentration dependent. HEK-Blue system was used to analyze neutralization of the LPS-induced inflammatory response. HEK-Blue cells were co-stimulated with 0.01, 0.03, and 0.1 mg/ml sHDL and 2 ng/ml LPS and incubated for 18 h. The activation of NF-κB reporter was quantified by measuring absorption at 650 nm (A-H). sHDL at 0.03 mg/ml were treated in presence of 2 ng/ml LPS and the activation of NF-κB reporter was quantified by measuring absorption at 650 nm (I).

FIG. 30: sHDL complexes enhance cholesterol efflux in a lipid component and concentration dependent. RAW 264.7 macrophages were labeled for 24 hours in growth medium containing 1 μCi of [³H] cholesterol/mL. The cells were then treated with 0.01 or 0.03 mg/ml sHDL complexes. Radioactive counts in media and cell fractions were measured by liquid scintillation counting.

### Definitions

To facilitate an understanding of the present invention, a number of terms and phrases are defined below:

As used herein, the term “we” or “our” refers to the inventors of the current patent application.

As used herein, the terms, “sepsis”, “sepsis related disorder”, LPS related disorder”, “condition associated with endotoxin”, “endotoxin associated disorder”, “endotoxin-related disorder”, or similar terms, describes any condition associated with LPS, e.g., a condition associated with bacteremia or introduction of lipopolysaccharide into the blood stream or onto an extra-gastrointestinal mucosal surface (e.g., the lung). Such disorders include, but are not limited to, endotoxin-related shock, endotoxin-related disseminated intravascular coagulation, endotoxin-related anemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leucopenia resulting from Gram-negative infection, hemodynamic shock and endotoxin-related pyresis.

The term “Gram-negative bacteria” is recognized in the art, and refers generally to bacteria that do not retain Gram stain (e.g., the deposition of a colored complex between crystal violet and iodine). In an exemplary Gram stain, cells are first fixed to a slide by heat and stained with a basic dye (e.g., crystal violet), which is taken up by all bacteria (i.e., both Gram-negative and Gram-positive). The slides are then treated with an iodine-KI mixture to fix the stain, washed with acetone or alcohol, and finally counterstained with a paler dye of different color (e.g., safranin). Gram-positive organisms retain the initial violet stain, while Gram-negative organisms are decolorized by the organic solvent and hence show the counterstain. Exemplary Gram-negative bacteria and cell lines include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Citrobacter spp., Chlamydia spp., Brucella spp., Pseudomonas spp., Helicobacter spp. and Vibrio spp.

The term, “viable non-toxic Gram-negative bacteria” refers to a viable Gram-negative bacterial strain comprising an outer membrane substantially free of LPS.

As used herein, the term “thrombosis” refers to the formation of a blood clot inside a blood vessel, obstructing the flow of blood through the circulatory system. In some embodiments, the thrombosis is “venous thrombosis” which is a blood clot that forms within a vein. In some embodiments, the thrombosis is “arterial thrombosis” which is a blood clot that forms within an artery.

As used here, the term “lipids” refer to fatty substances that are insoluble in water and include fats, oils, waxes, and related compounds. They may be either made in the blood (endogenous) or ingested in the diet (exogenous). Lipids are essential for normal body function and whether produced from an exogenous or endogenous source, they must be transported and then released for use by the cells. The production, transportation and release of lipids for use by the cells is referred to as lipid metabolism. While there are several classes of lipids, two major classes are cholesterol and triglycerides. Cholesterol may be ingested in the diet and manufactured by the cells of most organs and tissues in the body, primarily in the liver. Cholesterol can be found in its free form or, more often, combined with fatty acids as what is called cholesterol esters.

As used herein the term, “lipoproteins” refer to spherical compounds that are structured so that water-insoluble lipids are contained in a partially water-soluble shell. Depending on the type of lipoprotein, the contents include varying amounts of free and esterified cholesterol, triglycerides and apoproteins or apolipoproteins. There are five major types of lipoproteins, which differ in function and in their lipid and apoprotein content and are classified according to increasing density: (i) chylomicrons and chylomicron remnants, (ii) very low density lipoproteins (“VLDL”), (iii) intermediate-density lipoproteins (“IDL”), (iv) low-density lipoproteins (“LDL”), and (v) high-density lipoproteins (“HDL”). Cholesterol circulates in the bloodstream as particles associated with lipoproteins.

As used herein, the term “HDL” or “high density lipoprotein” refers to high-density lipoprotein. HDL comprises a complex of lipids and proteins in approximately equal amounts that functions as a transporter of cholesterol in the blood. HDL is mainly synthesized in and secreted from the liver and epithelial cells of the small intestine. Immediately after secretion, HDL is in a form of a discoidal particle containing apolipoprotein A-I (also called apoA-I) and phospholipid as its major constituents, and also called nascent HDL. This nascent HDL receives, in blood, free cholesterol from cell membranes of peripheral cells or produced in the hydrolysis course of other lipoproteins, and forms mature spherical HDL while holding, at its hydrophobic center, cholesterol ester converted from said cholesterol by the action of LCAT (lecithin cholesterol acyltransferase). HDL plays an extremely important role in a lipid metabolism process called “reverse cholesterol transport”, which takes, in blood, cholesterol out of peripheral tissues and transports it to the liver. High levels of HDL are associated with a decreased risk of atherosclerosis and coronary heart disease (CHD) as the reverse cholesterol transport is considered one of the major mechanisms for HDL's prophylactic action on atherosclerosis.

As used herein, the terms “synthetic HDL,” “sHDL,” “reconstituted HDL”, or “rHDL” refer to a particle structurally analogous to native HDL, composed of a lipid or lipids in association with at least one of the proteins of HDL, preferably Apo A-I or a mimetic thereof, and which exhibits all of the known physiological functions of HDL. Typically, the components of sHDL may be derived from blood, or produced by recombinant technology.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” or “therapeutic agent” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “solvent” refers to a medium in which a reaction is conducted. Solvents may be liquid but are not limited to liquid form. Solvent categories include but are not limited to nonpolar, polar, protic, and aprotic.

### DETAILED DESCRIPTION OF THE INVENTION

Experiments conducted during the course of developing embodiments for the present invention investigated the impact of phospholipid on the anti-inflammatory activities of HDL. Several studies emphasized the importance of the physical state of phospholipid on HDL that can potentially impact the functionality in cholesterol efflux but not in anti-inflammatory activities. Notably, liquid crystalline unsaturated phospholipids promote more efficient cholesterol acceptor than gel phase saturated phospholipids due to more fluid liquid crystalline lipids are capable of greater exogenous lipid molecule (e.g. cholesterol) insertion in contrast to relatively rigid gel phase phospholipids (32). To this extent, it was hypothesized that HDL with a fluid liquid crystalline lipid phase would also result in enhanced anti-inflammatory activities by accelerating the efflux of exogenous molecules (e.g. LPS and cholesterol) and accessibility to phospholipid.

To investigate such a hypothesis, rHDLs were prepared by complexing apoA-I mimetic peptide, 22A, with different phosphatidylcholines (PC). 22A peptide was chosen as it retains the biological activity of endogenous apolipoprotein A-I and has shown favorable safety and pharmacokinetics in human clinical trials (33, 34). PC is the largest class of phospholipids, comprising 33-45% of total HDL lipid mass (35) and is recognized to contribute to the potent anti-inflammatory effect of HDL (28). Thus, PC was selected as the HDL phospholipid composition with variations in fatty acid chain length and saturation to produce different fluidity of PC phase on rHDL due to distinct phase transition temperature (Tₘ) of each phospholipid (1-palmitoyl-2-oleoyl-phosphatidylcholine, POPC; 1,2-dimyristoyl-sn-glycero-3-phosphatidylcholine, DMPC; 1,2-dipalmitoyl-sn-glycero-3-phosphatidylcholine, DPPC; and 1,2-distearoyl-sn-glycero-3-phosphatidylcholine, DSPC) and examined the impact of fluidity of rHDL on its anti-inflammatory activities against LPS-induced inflammation in vitro and in vivo.

Such experiments demonstrated that changes in phospholipid composition of rHDL significantly enhance the anti-inflammatory activities due to different fluidity of rHDL. Fluid liquid crystalline phase rHDLs, particularly 22A-DMPC, most effectively modulated NF-κB signaling and pro-inflammatory mediators via TLR4 signaling as compared to rigid gel phase rHDL. Interestingly, only 22A-DMPC further reduced TLR4 recruitment into lipid rafts and promoted ATF3 expression which significantly modulated inflammatory activity with rHDL pre-treatment. Accordingly, 22A-DMPC improved mortality and protected organs from inflammatory injury in mice challenged with a lethal dose of LPS.

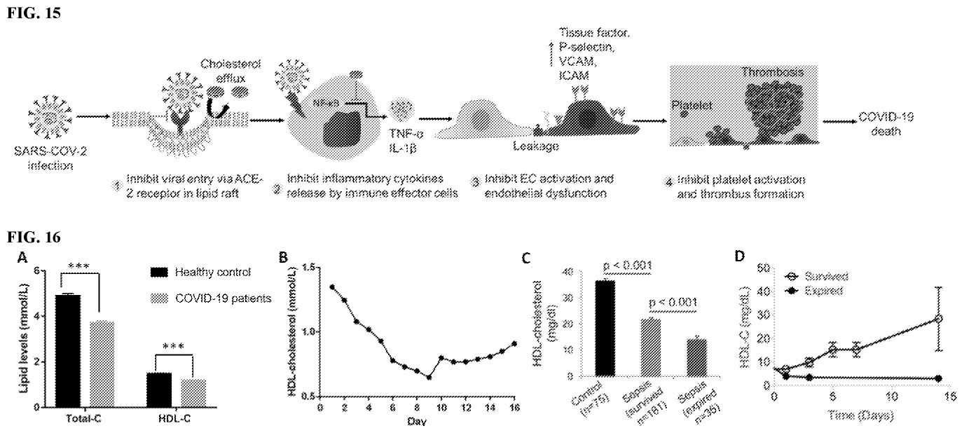

The pathogenic mechanism of SARS-CoV-2 infection and COVID-19 disease leading to pneumonia and ADRS seems to be particularly complex. The virus binds to the angiotensin-converting enzyme 2 (ACE2) receptor in humans, expressed in the endothelium, kidney, lung and heart. Viral infection leads to “cytokine storm”, the deadly uncontrolled systemic inflammatory response resulting from immune effector cell-medicated release of large quantity of pro-inflammatory cytokines (TNF-α, IFN-γ, IL-1β, IL-6). The proinflammatory cells results in endothelial cell activation, endothelial leakage and multiple organ failure. At the same time, platelet and fibrinogen are activated, leading to formation of multiple blood clots. Blood clots accumulate and form venous thrombosis, which is the main cause of septic shock symptoms such as diffuse intravascular coagulation (DIC). The venous thrombi flow to the lung and block pulmonary blood vessels, resulting in low oxygen blood oxygen, hypoxia, and sudden death. FIG. 15 schematically represents dynamics of COVID-19 pathology and a potential protective mechanism of sHDL infusions in COVID-19 through: 1) modulation of lipid raft composition resulting in reduced levels ACE2 and SARS-COV-2 virus cell entry; 2) inhibition of SARS-COV2 S protein induced NF-kB activation and reduction of proinflammatory cytokine release by immune effector cells; 3) inhibiting endothelial activation and dysfunction; 4) inhibition of platelet aggregation and thrombus formation.

Abundant clinical evidence has established an inverse correlation between high-density lipoprotein (HDL) cholesterol levels and the risk of thrombosis. Those clinical data suggest that raising HDL levels may be an important therapeutic strategy to reduce platelet hyperreactivity and the risk of thrombosis. In addition to its protective role in vascular endothelium, native HDL is reported to promote cholesterol efflux from platelets and change lipid raft organization in the platelet membrane, thus preventing platelet hyperreactivity. By mimicking the composition of native HDL, the present invention provides synthetic HDL (sHDL) infusion for reduction of platelet hyperactivity and prevention of thrombus formation. Indeed, experiments conducted during the course of developing embodiments for the present invention shows that sHDL, consisting of an apolipoprotein mimetic peptide and 1,2-dimyristoyl-sn-glycero-3-phosphocholine, significantly reduced platelet aggregation both in vitro and ex vivo as well as thrombus formation in vivo. Therefore, sHDL infusion provides a safer and more effective antithrombotic strategy to address the current clinical complications of antiplatelet agents by modifying the sHDL composition, understanding the potential mechanisms by which sHDL regulates platelet activity, and investigating the comprehensive in vivo performance of sHDL.

Accordingly, the present invention relates compositions comprising synthetic HDL (sHDL) nanoparticles, methods for synthesizing such sHDL nanoparticles, as well as systems and methods utilizing such sHDL nanoparticles (e.g., in diagnostic and/or therapeutic settings). In particular, the present invention provides compositions comprising sHDL nanoparticles for purposes of preventing, attenuating, and/or treating sepsis and sepsis related disorders in a subject, conditions and symptoms caused by a viral infection (e.g., COVID-19)) in a subject, and conditions and symptoms caused by thrombosis in a subject.

As noted, the sHDL nanoparticles of the present invention are useful in treating sepsis and sepsis related disorders.

Examples of sepsis related disorders include, any condition associated with bacteremia or introduction of lipopolysaccharide into the blood stream or onto an extra-gastrointestinal mucosal surface (e.g., the lung). Such disorders include, but are not limited to, endotoxin-related shock, endotoxin-related disseminated intravascular coagulation, endotoxin-related anemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leucopenia resulting from Gram-negative infection, hemodynamic shock and endotoxin-related pyresis.

Examples of a viral infection include any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having M^{pro} protease activity and/or expression. In some embodiments, the viral infection is a SARS-CoV-2 related viral infection (e.g., COVID-19).

In some embodiments, the conditions and symptoms caused by thrombosis are related to a venous thrombosis. In some embodiments, the conditions and symptoms caused by thrombosis are related to an arterial thrombosis.

In some embodiments, thrombosis is a feature of an underlying disease or condition. Non-limiting examples of such disease or condition include acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, thromboembolic stroke, systemic embolism, ischemic stroke, venous thromboembolism, atrial fibrillation, non-valvular atrial fibrillation, atrial flutter, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the conditions and symptoms caused by thrombosis are selected from the group consisting of embolic stroke, thrombotic stroke, venous thrombosis, deep venous thrombosis, acute coronary syndrome, and myocardial infarction.

The present invention is not limited to a particular method or technique for preventing, attenuating, and/or treating sepsis and sepsis related disorders in a subject, conditions and symptoms caused by a viral infection (e.g., COVID-19) in a subject, and conditions and symptoms caused by thrombosis in a subject.

In some embodiments, the methods involve administering to a subject (e.g., a human subject suffering from or such a condition) a therapeutically effective amount of a composition comprising a sHDL nanoparticle as described herein.

In some embodiments, the administering to the subject a therapeutically effective amount of a composition comprising a sHDL comprises a continuous infusion of sHDL and/or non-continuous infusions of sHDL.

In some embodiments involving preventing, attenuating, and/or treating sepsis and sepsis related disorders in a subject, administration of the sHDL nanoparticle results in attenuation of inflammatory activity in the subject through, for example, suppression of NF-kB signaling, regulating TLR4 recruitment into lipid rafts, promoting ATF-3 expression, protecting organs from organ failure, and neutralization of LPS.

In some embodiments involving preventing, attenuating, and/or treating conditions and symptoms caused by a viral infection (e.g., COVID-19), administration of the sHDL nanoparticle results in modulation of lipid raft composition resulting in reduced levels ACE2 and SARS-COV-2 virus cell entry; inhibition of SARS-COV2 S protein induced NF-kB activation and reduction of proinflammatory cytokine release by immune effector cells; and inhibiting endothelial activation and dysfunction.

In some embodiments involving preventing, attenuating, and/or treating conditions and symptoms caused by thrombosis, administration of the sHDL nanoparticle results reduction of platelet activity, prevention of thrombus formation, and reduction of platelet aggregation.

The present invention also includes methods involving co-administration of the sHDL nanoparticles as described herein with one or more additional active agents. Indeed, it is a further aspect of this invention to provide methods for enhancing prior art therapies and/or pharmaceutical compositions by co-administering the sHDL nanoparticles of this invention. In co-administration procedures, the agents may be administered concurrently or sequentially. In some embodiments, the sHDL nanoparticles described herein are administered prior to the other active agent(s).

The agent or agents to be co-administered depends on the type of condition being treated. The additional agents to be co-administered can be any of the well-known agents in the art, including, but not limited to, those that are currently in clinical use.

For example, when the condition being treated is sepsis or a sepsis related disorder, the additional agent includes, but are not limited to, alpha-/beta-adrenergic agonists (e.g., norepinephrine, dopamine, dobutamine, epinephrine, vasopressin, phenylephrine), isotonic crystalloids, albumin, antibiotics (e.g., cefotaxime, ticarcillin-clavulanate, piperacillin-tazobactam, imipenem-cilastatin, meropenem, clindamycin, metronidazole, ceftriaxone, ciprofloxacin, cefepime, levofloxacin, vancomycin), and corticosteroids (e.g., hydrocortisone, dexamethasone).

For example, when the condition being treated is conditions and symptoms caused by a viral infection (e.g., COVID-19), the additional agent includes, but are not limited to, remdesivir, dexamethasone, and hydroxychloroquine.

For example, when the condition being treated is conditions and symptoms caused by thrombosis, the additional agent includes, but are not limited to, heparin; tPA; anistreplase; streptokinase; urokinase; a coumadin; warfarin; idraparinux; fondaparinux; aspririn; an adenosine diphosphate receptor inhibitor; a phosphodiesterase inhibitor; a glycoprotein IIB/IIA inhibitor; an adenosine reuptake inhibitor; and a thromboxane receptor antagonist.

The present invention is not limited to specific types or kinds of sHDL nanoparticles for purposes of preventing, attenuating, and/or treating sepsis and sepsis related disorders in a subject.

In some embodiments, the average particle size of the sHDL nanoparticle is between 6-20 nm. In some embodiments, the average particle size of the sHDL nanoparticle is between 7-12 nm. In some embodiments, the ratio of HDL apolipoprotein to phospholipid is at or between 1:1 to 1:4 wt/wt.

Generally, sHDL nanoparticles are composed of a mixture of HDL apolipoprotein and an amphipathic lipid.

Examples of suitable apolipoproteins include, but are not limited to, preproapolipoprotein forms of ApoA-I, ApoA-II, ApoA-IV, ApoA-V and ApoE; pro- and mature forms of human ApoA-I, ApoA-II, ApoA-IV, and ApoE; and active polymorphic forms, isoforms, variants and mutants as well as truncated forms, the most common of which are ApoA-IM (ApoA-IM) and ApoA-IP (ApoA-IP). Apolipoproteins mutants containing cysteine residues are also known, and can also be used (see, e.g., U.S. 2003/0181372). The apolipoproteins may be in the form of monomers or dimers, which may be homodimers or heterodimers. For example, homo- and heterodimers (where feasible) of pro- and mature ApoA-I (Duverger et al., 1996, Arterioscler. Thromb. Vasc. Biol. 16(12):1424-29), ApoA-IM (Franceschini et al., 1985, J. Biol. Chem. 260:1632-35), ApoA-IP (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), ApoE (McLean et al., 1983, J. Biol. Chem. 258(14):8993-9000), ApoJ and ApoH may be used. The apolipoproteins may include residues corresponding to elements that facilitate their isolation, such as His tags, or other elements designed for other purposes, so long as the apolipoprotein retains some biological activity when included in a complex.

Such apolipoproteins can be purified from animal sources (and in particular from human sources) or produced recombinantly as is well-known in the art, see, e.g., Chung et al., 1980, J. Lipid Res. 21(3):284-91; Cheung et al., 1987, J. Lipid Res. 28(8):913-29 (see, also, U.S. Pat. Nos. 5,059,528, 5,128,318, 6,617,134, and U.S. Publication Nos. 20002/0156007, 2004/0067873, 2004/0077541, and 2004/0266660).

Non-limiting examples of peptides and peptide analogs that correspond to apolipoproteins, as well as agonists that mimic the activity of ApoA-I, ApoA-I_{M}, ApoA-II, ApoA-IV, and ApoE, that are suitable for use as apolipoproteins in the charged complexes and compositions described herein are disclosed in U.S. Pat. Nos. 6,004,925, 6,037,323 and 6,046,166 (issued to Dasseux et al.), U.S. Pat. No. 5,840,688 (issued to Tso), U.S. publications 2004/0266671, 2004/0254120, 2003/0171277 and 2003/0045460 (to Fogelman), and U.S. publication 2003/0087819 (to Bielicki), the disclosures of which are incorporated herein by reference in their entireties. These peptides and peptide analogues can be composed of L-amino acid or D-amino acids or mixture of L- and D-amino acids. They may also include one or more non-peptide or amide linkages, such as one or more well-known peptide/amide isosteres. Such “peptide and/or peptide mimetic” apolipoproteins can be synthesized or manufactured using any technique for peptide synthesis known in the art, including, e.g., the techniques described in U.S. Pat. Nos. 6,004,925, 6,037,323 and 6,046,166.

In some embodiments, HDL apolipoproteins include, for example apolipoprotein A-I (apo A-I), apolipoprotein A-II (apo A-II), apolipoprotein A4 (apo A4), apolipoprotein Cs (apo Cs), apolipoprotein M (apo M), and apolipoprotein E (apo E). Preferably, the carrier particles are composed of Apo A-I or Apo A-II, however the use of other lipoproteins including apolipoprotein A4, apolipoprotein Cs or apolipoprotein E may be used alone or in combination to formulate carrier particle mixtures for delivery of therapeutic agents. In some embodiments, the HDL apolipoprotein is selected from preproapoliprotein, preproApoA-I, proApoA-I, ApoA-I, preproApoA-II, proApoA-II, ApoA-II, apolipoprotein A-II xxx (apo A-II-xxx), preproApoA-IV, proApoA-IV, ApoA-IV, ApoA-V, preproApoE, proApoE, ApoE, preproApoA-lMilano, proApoA-IMilano ApoA-lMilano preproApoA-IParis, proApoA-IParis, and ApoA-IParis and peptide mimetics of these proteins mixtures thereof. In some embodiments, mimetics of such HDL apolipoproteins are used.

ApoA-I is synthesized by the liver and small intestine as preproapolipoprotein which is secreted as a proprotein that is rapidly cleaved to generate a mature polypeptide having 243 amino acid residues. ApoA-I consists mainly of 6 to 8 different 22 amino acid repeats spaced by a linker moiety which is often proline, and in some cases consists of a stretch made up of several residues. ApoA-I forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL₃ and HDL₂). Most HDL in the circulating population contain both ApoA-I and ApoA-II (the second major HDL protein). However, the fraction of HDL containing only ApoA-I (referred to herein as the AI-HDL fraction) is more effective in reverse cholesterol transport.

In some embodiments, ApoA-I agonists or mimetics are provided. In some embodiments, such ApoA-I mimetics are capable of forming amphipathic α-helices that mimic the activity of ApoA-I, and have specific activities approaching or exceeding that of the native molecule. In some, the ApoA-I mimetics are peptides or peptide analogues that: form amphipathic helices (in the presence of lipids), bind lipids, form pre-β-like or HDL-like complexes, activate lecithin:cholesterol acyltransferase (LCAT), increase serum levels of HDL fractions, and promote cholesterol efflux.

The present invention is not limited to use of a particular ApoA-I mimetic. In some embodiments, any of the ApoA-I mimetics described in Srinivasa, et al., 2014 Curr. Opinion Lipidology Vol. 25(4): 304-308 are utilized. In some embodiments, any of the ApoA-I mimetics described in U.S. Patent Application Publication Nos. 20110046056 and 20130231459 are utilized.

In some embodiments, the “22A” ApoA-I mimetic is used (PVLDLFRELLNELLEALKQKLK) (SEQ ID NO: 4) (see, e.g., U.S. Pat. No. 7,566,695). In some embodiments, any of the following ApoA-I mimetics shown in Table 1 as described in U.S. Pat. No. 7,566,695 are utilized:

TABLE 1
ApoA-I mimetics

| SEQ ID NO: | AMINO ACID SEQUENCE |
|---|---|
| (SEQ ID NO: 1) | PVLDLFRELLNELLEZLKQKLK |
| (SEQ ID NO: 2) | GVLDLFRELLNELLEALKQKLKK |
| (SEQ ID NO: 3) | PVLDLFRELLNELLEWLKQKLK |
| (SEQ ID NO: 4) | PVLDLFRELLNELLEALKQKLK |
| (SEQ ID NO: 5) | pVLDLFRELLNELLEALKQKLKK |
| (SEQ ID NO: 6) | PVLDLFRELLNEXLEALKQKLK |
| (SEQ ID NO: 7) | PVLDLFKELLNELLEALKQKLK |
| (SEQ ID NO: 8) | PVLDLFRELLNEGLEALKQKLK |
| (SEQ ID NO: 9) | PVLDLFRELGNELLEALKQKLK |
| (SEQ ID NO: 10) | PVLDLFRELLNELLEAZKQKLK |
| (SEQ ID NO: 11) | PVLDLFKELLQELLEALKQKLK |
| (SEQ ID NO: 12) | PVLDLFRELLNELLEAGKQKLK |
| (SEQ ID NO: 13) | GVLDLFRELLNEGLEALKQKLK |
| (SEQ ID NO: 14) | PVLDLFRELLNELLEALOQOLO |
| (SEQ ID NO: 15) | PVLDLFRELWNELLEALKQKLK |
| (SEQ ID NO: 16) | PVLDLLRELLNELLEALKQKLK |
| (SEQ ID NO: 17) | PVLELFKELLQELLEALKQKLK |
| (SEQ ID NO: 18) | GVLDLFRELLNELLEALKQKLK |
| (SEQ ID NO: 19) | pVLDLFRELLNEGLEALKQKLK |
| (SEQ ID NO: 20) | PVLDLFREGLNELLEALKQKLK |
| (SEQ ID NO: 21) | pVLDLFRELLNELLEALKQKLK |
| (SEQ ID NO: 22) | PVLDLFRELLNELLEGLKQKLK |
| (SEQ ID NO: 23) | PLLELFKELLQELLEALKQKLK |
| (SEQ ID NO: 24) | PVLDLFRELLNELLEALQKKLK |
| (SEQ ID NO: 25) | PVLDFFRELLNEXLEALKQKLK |
| (SEQ ID NO: 26) | PVLDLFRELLNELLELLKQKLK |
| (SEQ ID NO: 27) | PVLDLFRELLNELZEALKQKLK |
| (SEQ ID NO: 28) | PVLDLFRELLNELWEALKQKLK |
| (SEQ ID NO: 29) | AVLDLFRELLNELLEALKQKLK |
| (SEQ ID NO: 30) | PVLDLPRELLNELLEALKQKLK¹ |
| (SEQ ID NO: 31) | PVLDLFLELLNEXLEALKQKLK |
| (SEQ ID NO: 32) | XVLDLFRELLNELLEALKQKLK |
| (SEQ ID NO: 33) | PVLDLFREKLNELLEALKQKLK |
| (SEQ ID NO: 34) | PVLDZFRELLNELLEALKQKLK |
| (SEQ ID NO: 35) | PVLDWFRELLNELLEALKQKLK |
| (SEQ ID NO: 36) | PLLELLKELLQELLEALKQKLK |
| (SEQ ID NO: 37) | PVLDLFREWLNELLEALKQKLK |
| (SEQ ID NO: 38) | PVLDLFRELLNEXLEAWKQKLK |
| (SEQ ID NO: 39) | PVLDLFRELLEELLKALKKKLK |
| (SEQ ID NO: 40) | PVLDLFNELLRELLEALQKKLK |
| (SEQ ID NO: 41) | PVLDLWRELLNEXLEALKQKLK |
| (SEQ ID NO: 42) | PVLDEFREKLNEXWEALKQKLK |
| (SEQ ID NO: 43) | PVLDEFREKLWEXLEALKQKLK |
| (SEQ ID NO: 44) | pvldefreklneXlealkqklk |
| (SEQ ID NO: 45) | PVLDEFREKLNEXLEALKQKLK |
| (SEQ ID NO: 46) | PVLDLFREKLNEXLEALKQKLK |
| (SEQ ID NO: 47) | ~VLDLFRELLNEGLEALKQKLK |
| (SEQ ID NO: 48) | pvLDLFRELLNELLEALKQKLK |
| (SEQ ID NO: 49) | PVLDLFRNLLEKLLEALEQKLK |
| (SEQ ID NO: 50) | PVLDLFRELLWEXLEALKQKLK |
| (SEQ ID NO: 51) | PVLDLFWELLNEXLEALKQKLK |
| (SEQ ID NO: 52) | PVWDEFREKLNEXLEALKQKLK |
| (SEQ ID NO: 53) | WVLDLFRELLNELLEALKQKLK |
| (SEQ ID NO: 54) | PVLDLFRELLNEWLEALKQKLK |
| (SEQ ID NO: 55) | P~~~LFRELLNELLEALKQKLK |
| (SEQ ID NO: 56) | PVLDLFRELLNELLEALKQKKK |
| (SEQ ID NO: 57) | PVLDLFRNLLEELLKALEQKLK |
| (SEQ ID NO: 58) | PVLDEFREKLNEXLEALKQKL~ |
| (SEQ ID NO: 59) | LVLDLFRELLNELLEALKQKLK |
| (SEQ ID NO: 60) | PVLDLFRELLNELLEALKQ~~~ |
| (SEQ ID NO: 61) | PVLDEFRWKLNEXLEALKQKLK |
| (SEQ ID NO: 62) | PVLDEWREKLNEXLEALKQKLK |
| (SEQ ID NO: 63) | PVLDFFREKLNEXLEALKQKLK |
| (SEQ ID NO: 64) | PWLDEFREKLNEXLEALKQKLK |
| (SEQ ID NO: 65) | ~VLDEFREKLNEXLEALKQKLK |
| (SEQ ID NO: 66) | PVLDLFRNLLEELLEALQKKLK |
| (SEQ ID NO: 67) | ~VLDLFRELLNELLEALKQKLK |
| (SEQ ID NO: 68) | PVLDEFRELLKEXLEALKQKLK |
| (SEQ ID NO: 69) | PVLDEFRKKLNEXLEALKQKLK |
| (SEQ ID NO: 70) | PVLDEFRELLYEXLEALKQKLK |
| (SEQ ID NO: 71) | PVLDEFREKLNELXEALKQKLK |
| (SEQ ID NO: 72) | PVLDLFRELLNEXLWALKQKLK |
| (SEQ ID NO: 73) | PVLDEFWEKLNEXLEALKQKLK |
| (SEQ ID NO: 74) | PVLDKFREKLNEXLEALKQKLK |
| (SEQ ID NO: 75) | PVLDEFREKLNEELEALKQKLK |
| (SEQ ID NO: 76) | PVLDEFRELLFEXLEALKQKLK |
| (SEQ ID NO: 77) | PVLDEFREKLNKXLEALKQKLK |
| (SEQ ID NO: 78) | PVLDEFRDKLNEXLEALKQKLK |
| (SEQ ID NO: 79) | PVLDEFRELLNELLEALKQKLK |
| (SEQ ID NO: 80) | PVLDLFERLLNELLEALQKKLK |
| (SEQ ID NO: 81) | PVLDEFREKLNWXLEALKQKLK |
| (SEQ ID NO: 82) | ~~LDEFREKLNEXLEALKQKLK |
| (SEQ ID NO: 83) | PVLDEFREKLNEXLEALWQKLK |
| (SEQ ID NO: 84) | PVLDEFREKLNELLEALKQKLK |
| (SEQ ID NO: 85) | P~LDLFRELLNELLEALKQKLK |
| (SEQ ID NO: 86) | PVLELFERLLDELLNALQKKLK |
| (SEQ ID NO: 87) | pllellkellqellealkqklk |
| (SEQ ID NO: 88) | PVLDKFRELLNEXLEALKQKLK |
| (SEQ ID NO: 89) | PVLDEFREKLNEXLWALKQKLK |
| (SEQ ID NO: 90) | ~~~DEFREKLNEXLEALKQKLK |
| (SEQ ID NO: 91) | PVLDEFRELLNEXLEALKQKLK |
| (SEQ ID NO: 92) | PVLDEFRELYNEXLEALKQKLK |
| (SEQ ID NO: 93) | PVLDEFREKLNEXLKALKQKLK |
| (SEQ ID NO: 94) | PVLDEFREKLNEALEALKQKLK |
| (SEQ ID NO: 95) | PVLDLFRELLNLXLEALKQKLK |
| (SEQ ID NO: 96) | pvldlfrellneXlealkqklk |
| (SEQ ID NO: 97) | PVLDLFRELLNELLE~~~~~~~ |
| (SEQ ID NO: 98) | PVLDLFRELLNEELEALKQKLK |
| (SEQ ID NO: 99) | KLKQKLAELLENLLERFLDLVP |
| (SEQ ID NO: 100) | pvldlfrellnellealkqklk |
| (SEQ ID NO: 101) | PVLDLFRELLNWXLEALKQKLK |
| (SEQ ID NO: 102) | PVLDLFRELLNLXLEALKEKLK |
| (SEQ ID NO: 103) | PVLDEFRELLNEELEALKQKLK |
| (SEQ ID NO: 104) | P~~~~~~~LLNELLEALKQKLK |
| (SEQ ID NO: 105) | PAADAFREAANEAAEAAKQKAK |
| (SEQ ID NO: 106) | PVLDLFREKLNEELEALKQKLK |
| (SEQ ID NO: 107) | klkqklaellenllerfldlvp |
| (SEQ ID NO: 108) | PVLDLFRWLLNEXLEALKQKLK |
| (SEQ ID NO: 109) | PVLDEFREKLNERLEALKQKLK |
| (SEQ ID NO: 110) | PVLDEFREKLNEXXEALKQKLK |
| (SEQ ID NO: 111) | PVLDEFREKLWEXWEALKQKLK |
| (SEQ ID NO: 112) | PVLDEFREKLNEXSEALKQKLK |
| (SEQ ID NO: 113) | PVLDEFREKLNEPLEALKQKLK |
| (SEQ ID NO: 114) | PVLDEFREKLNEXMEALKQKLK |
| (SEQ ID NO: 115) | PKLDEFREKLNEXLEALKQKLK |
| (SEQ ID NO: 116) | PHLDEFREKLNEXLEALKQKLK |
| (SEQ ID NO: 117) | PELDEFREKLNEXLEALKQKLK |
| (SEQ ID NO: 118) | PVLDEFREKLNEXLEALEQKLK |
| (SEQ ID NO: 119) | PVLDEFREKLNEELEAXKQKLK |
| (SEQ ID NO: 120) | PVLDEFREKLNEELEXLKQKLK |
| (SEQ ID NO: 121) | PVLDEFREKLNEELEALWQKLK |
| (SEQ ID NO: 122) | PVLDEFREKLNEELEWLKQKLK |
| (SEQ ID NO: 123) | QVLDLFRELLNELLEALKQKLK |
| (SEQ ID NO: 124) | PVLDLFOELLNELLEALOQOLO |
| (SEQ ID NO: 125) | NVLDLFRELLNELLEALKQKLK |
| (SEQ ID NO: 126) | PVLDLFRELLNELGEALKQKLK |
| (SEQ ID NO: 127) | PVLDLFRELLNELLELLKQKLK |
| (SEQ ID NO: 128) | PVLDLFRELLNELLEFLKQKLK |
| (SEQ ID NO: 129) | PVLELFNDLLRELLEALQKKLK |
| (SEQ ID NO: 130) | PVLELFNDLLRELLEALKQKLK |
| (SEQ ID NO: 131) | PVLELFKELLNELLDALRQKLK |
| (SEQ ID NO: 132) | PVLDLFRELLENLLEALQKKLK |
| (SEQ ID NO: 133) | PVLELFERLLEDLLQALNKKLK |
| (SEQ ID NO: 134) | PVLELFERLLEDLLKALNOKLK |
| (SEQ ID NO: 135) | DVLDLFRELLNELLEALKQKLK |
| (SEQ ID NO: 136) | PALELFKDLLQELLEALKQKLK |
| (SEQ ID NO: 137) | PVLDLFRELLNEGLEAZKQKLK |
| (SEQ ID NO: 138) | PVLDLFRELLNEGLEWLKQKLK |
| (SEQ ID NO: 139) | PVLDLFRELWNEGLEALKQKLK |
| (SEQ ID NO: 140) | PVLDLFRELLNEGLEALOQOLO |
| (SEQ ID NO: 141) | PVLDFFRELLNEGLEALKQKLK |
| (SEQ ID NO: 142) | PVLELFRELLNEGLEALKQKLK |
| (SEQ ID NO: 143) | PVLDLFRELLNEGLEALKQKLK* |
| (SEQ ID NO: 144) | pVLELFENLLERLLDALQKKLK |
| (SEQ ID NO: 145) | GVLELFENLLERLLDALQKKLK |
| (SEQ ID NO: 146) | PVLELFENLLERLLDALQKKLK |
| (SEQ ID NO: 147) | PVLELFENLLERLFDALQKKLK |
| (SEQ ID NO: 148) | PVLELFENLLERLGDALQKKLK |
| (SEQ ID NO: 149) | PVLELFENLWERLLDALQKKLK |
| (SEQ ID NO: 150) | PLLELFENLLERLLDALQKKLK |
| (SEQ ID NO: 151) | PVLELFENLGERLLDALQKKLK |
| (SEQ ID NO: 152) | PVFELFENLLERLLDALQKKLK |
| (SEQ ID NO: 153) | AVLELFENLLERLLDALQKKLK |
| (SEQ ID NO: 154) | PVLELFENLLERGLDALQKKLK |
| (SEQ ID NO: 155) | PVLELFLNLWERLLDALQKKLK |
| (SEQ ID NO: 156) | PVLELFLNLLERLLDALQKKLK |
| (SEQ ID NO: 157) | PVLEFFENLLERLLDALQKKLK |
| (SEQ ID NO: 158) | PVLELFLNLLERLLDWLQKKLK |
| (SEQ ID NO: 159) | PVLDLFENLLERLLDALQKKLK |
| (SEQ ID NO: 160) | PVLELFENLLERLLDWLQKKLK |
| (SEQ ID NO: 161) | PVLELFENLLERLLEALQKKLK |
| (SEQ ID NO: 162) | PVLELFENWLERLLDALQKKLK |
| (SEQ ID NO: 163) | PVLELFENLLERLWDALQKKLK |
| (SEQ ID NO: 164) | PVLELFENLLERLLDAWQKKLK |
| (SEQ ID NO: 165) | PVLELFENLLERLLDLLQKKLK |
| (SEQ ID NO: 166) | PVLELFLNLLEKLLDALQKKLK |
| (SEQ ID NO: 167) | PVLELFENGLERLLDALQKKLK |
| (SEQ ID NO: 168) | PVLELFEQLLEKLLDALQKKLK |
| (SEQ ID NO: 169) | PVLELFENLLEKLLDALQKKLK |
| (SEQ ID NO: 170) | PVLELFENLLEOLLDALQOOLO |
| (SEQ ID NO: 171) | PVLELFENLLEKLLDLLQKKLK |
| (SEQ ID NO: 172) | PVLELFLNLLERLGDALQKKLK |
| (SEQ ID NO: 173) | PVLDLFDNLLDRLLDLLNKKLK |
| (SEQ ID NO: 174) | pvlelfenllerlldalqkklk |
| (SEQ ID NO: 175) | PVLELFENLLERLLELLNKKLK |
| (SEQ ID NO: 176) | PVLELWENLLERLLDALQKKLK |
| (SEQ ID NO: 177) | GVLELFLNLLERLLDALQKKLK |
| (SEQ ID NO: 178) | PVLELFDNLLEKLLEALQKKLR |
| (SEQ ID NO: 179) | PVLELFDNLLERLLDALQKKLK |
| (SEQ ID NO: 180) | PVLELFDNLLDKLLDALQKKLR |
| (SEQ ID NO: 181) | PVLELFENLLERWLDALQKKLK |
| (SEQ ID NO: 182) | PVLELFENLLEKLLEALQKKLK |
| (SEQ ID NO: 183) | PLLELFENLLEKLLDALQKKLK |
| (SEQ ID NO: 184) | PVLELFLNLLERLLDAWQKKLK |
| (SEQ ID NO: 185) | PVLELFENLLERLLDALQOOLO |
| (SEQ ID NO: 186) | PVLELFEQLLERLLDALQKKLK |
| (SEQ ID NO: 187) | PVLELFENLLERLLDALNKKLK |
| (SEQ ID NO: 188) | PVLELFENLLDRLLDALQKKLK |
| (SEQ ID NO: 189) | DVLELFENLLERLLDALQKKLK |
| (SEQ ID NO: 190) | PVLEFWDNLLDKLLDALQKKLR |
| (SEQ ID NO: 191) | PVLDLLRELLEELKQKLK* |
| (SEQ ID NO: 192) | PVLDLFKELLEELKQKLK* |
| (SEQ ID NO: 193) | PVLDLFRELLEELKQKLK* |
| (SEQ ID NO: 194) | PVLELFRELLEELKQKLK* |
| (SEQ ID NO: 195) | PVLELFKELLEELKQKLK* |
| (SEQ ID NO: 196) | PVLDLFRELLEELKNKLK* |
| (SEQ ID NO: 197) | PLLDLFRELLEELKQKLK* |
| (SEQ ID NO: 198) | GVLDLFRELLEELKQKLK* |
| (SEQ ID NO: 199) | PVLDLFRELWEELKQKLK* |
| (SEQ ID NO: 200) | NVLDLFRELLEELKQKLK* |
| (SEQ ID NO: 201) | PLLDLFKELLEELKQKLK* |
| (SEQ ID NO: 202) | PALELFKDLLEELRQKLR* |
| (SEQ ID NO: 203) | AVLDLFRELLEELKQKLK* |
| (SEQ ID NO: 204) | PVLDFFRELLEELKQKLK* |
| (SEQ ID NO: 205) | PVLDLFREWLEELKQKLK* |
| (SEQ ID NO: 206) | PLLELLKELLEELKQKLK* |
| (SEQ ID NO: 207) | PVLELLKELLEELKQKLK* |
| (SEQ ID NO: 208) | PALELFKDLLEELRQRLK* |
| (SEQ ID NO: 209) | PVLDLFRELLNELLQKLK |
| (SEQ ID NO: 210) | PVLDLFRELLEELKQKLK |
| (SEQ ID NO: 211) | PVLDLFRELLEELOQOLO* |
| (SEQ ID NO: 212) | PVLDLFOELLEELOQOLK* |
| (SEQ ID NO: 213) | PALELFKDLLEEFRQRLK* |
| (SEQ ID NO: 214) | pVLDLFRELLEELKQKLK* |
| (SEQ ID NO: 215) | PVLDLFRELLEEWKQKLK* |
| (SEQ ID NO: 216) | PVLELFKELLEELKQKLK |
| (SEQ ID NO: 217) | PVLDLFRELLELLKQKLK |
| (SEQ ID NO: 218) | PVLDLFRELLNELLQKLK* |
| (SEQ ID NO: 219) | PVLDLFRELLNELWQKLK |
| (SEQ ID NO: 220) | PVLDLFRELLEELQKKLK |
| (SEQ ID NO: 221) | DVLDLFRELLEELKQKLK* |
| (SEQ ID NO: 222) | PVLDAFRELLEALLQLKK |
| (SEQ ID NO: 223) | PVLDAFRELLEALAQLKK |
| (SEQ ID NO: 224) | PVLDLFREGWEELKQKLK |
| (SEQ ID NO: 225) | PVLDAFRELAEALAQLKK |
| (SEQ ID NO: 226) | PVLDAFRELGEALLQLKK |
| (SEQ ID NO: 227) | PVLDLFRELGEELKQKLK* |
| (SEQ ID NO: 228) | PVLDLFREGLEELKQKLK* |
| (SEQ ID NO: 229) | PVLDLFRELLEEGKQKLK* |
| (SEQ ID NO: 230) | PVLELFERLLEDLQKKLK |
| (SEQ ID NO: 231) | PVLDLFRELLEKLEQKLK |
| (SEQ ID NO: 232) | PLLELFKELLEELKQKLK* |
| (SEQ ID NO: 233) | LDDLLQKWAEAFNQLLKK |
| (SEQ ID NO: 234) | EWLKAFYEKVLEKLKELF* |
| (SEQ ID NO: 235) | EWLEAFYKKVLEKLKELF* |
| (SEQ ID NO: 236) | DWLKAFYDKVAEKLKEAF* |
| (SEQ ID NO: 237) | DWFKAFYDKVFEKFKEFF |
| (SEQ ID NO: 238) | GIKKFLGSIWKFIKAFVG |
| (SEQ ID NO: 239) | DWFKAFYDKVAEKFKEAF |
| (SEQ ID NO: 240) | DWLKAFYDKVAEKLKEAF |
| (SEQ ID NO: 241) | DWLKAFYDKVFEKFKEFF |
| (SEQ ID NO: 242) | EWLEAFYKKVLEKLKELP |
| (SEQ ID NO: 243) | DWFKAFYDKFFEKFKEFF |
| (SEQ ID NO: 244) | EWLKAFYEKVLEKLKELF |
| (SEQ ID NO: 245) | EWLKAEYEKVEEKLKELF* |
| (SEQ ID NO: 246) | EWLKAEYEKVLEKLKELF* |
| (SEQ ID NO: 247) | EWLKAFYKKVLEKLKELF* |
| (SEQ ID NO: 248) | PVLDLFRELLEQKLK* |
| (SEQ ID NO: 249) | PVLDLFRELLEELKQK* |
| (SEQ ID NO: 250) | PVLDLFRELLEKLKQK* |
| (SEQ ID NO: 251) | PVLDLFRELLEKLQK* |
| (SEQ ID NO: 252) | PVLDLFRELLEALKQK* |
| (SEQ ID NO: 253) | PVLDLFENLLERLKQK* |
| (SEQ ID NO: 254) | PVLDLFRELLNELKQK* |

*indicates peptides that are N-terminal acetylated and C-terminal amidated; indicates peptides that are N-terminal dansylated; sp indicates peptides that exhibited solubility problems under the experimental conditions; X is Aib; Z is Nal; O is Orn; He (%) designates percent helicity; mics designates micelles; and ~ indicates deleted amino acids.

In some embodiments, an ApoA-I mimetic having the following sequence as described in U.S. Pat. No. 6,743,778 is utilized: Asp Trp Leu Lys Ala Phe Tyr Asp Lys Val Ala Glu Lys Leu Lys Glu Ala Phe (SEQ ID NO: 256).

In some embodiments, any of the following ApoA-I mimetics shown in Table 2 as described in U.S. Patent Application Publication No. 2003/0171277 are utilized:

TABLE 2

| SEQ ID NO: | AMINO ACID SEQUENCE |
|---|---|
| (SEQ ID NO: 256) | D-W-L-K-A-F-Y-D-K-V-A-E-K-L-K-E-A-F |
| (SEQ ID NO: 257) | Ac-D-W-L-K-A-F-Y-D-K-V-A-E-K-L-K-E-A-F-NH₂ |
| (SEQ ID NO: 258) | Ac-D-W-F-K-A-F-Y-D-K-V-A-E-K-L-K-E-A-F-NH₂ |
| (SEQ ID NO: 259) | Ac-D-W-L-K-A-F-Y-D-K-V-A-E-K-F-K-E-A-F-NH₂ |
| (SEQ ID NO: 260) | Ac-D-W-F-K-A-F-Y-D-K-V-A-E-K-F-K-E-A-F-NH₂ |
| (SEQ ID NO: 261) | Ac-D-W-L-K-A-F-Y-D-K-V-F-E-K-F-K-E-F-F-NH₂ |
| (SEQ ID NO: 262) | Ac-D-W-L-K-A-F-Y-D-K-F-F-E-K-F-K-E-F-F-NH₂ |
| (SEQ ID NO: 263) | Ac-D-W-F-K-A-F-Y-D-K-F-F-E-K-F-K-E-F-F-NH₂ |
| (SEQ ID NO: 264) | Ac-D-W-L-K-A-F-Y-D-K-V-A-E-K-L-K-E-F-F-NH₂ |
| (SEQ ID NO: 265) | Ac-D-W-L-K-A-F-Y-D-K-V-F-E-K-F-K-E-A-F-NH₂ |
| (SEQ ID NO: 266) | Ac-D-W-L-K-A-F-Y-D-K-V-F-E-K-L-K-E-F-F-NH₂ |
| (SEQ ID NO: 267) | Ac-D-W-L-K-A-F-Y-D-K-V-A-E-K-F-K-E-F-F-NH₂ |
| (SEQ ID NO: 268) | Ac-D-W-L-K-A-F-Y-D-K-V-F-E-K-F-K-E-F-F-NH₂ |
| (SEQ ID NO: 269) | Ac-E-W-L-K-L-F-Y-E-K-V-L-E-K-F-K-E-A-F-NH₂ |
| (SEQ ID NO: 270) | Ac-E-W-L-K-A-F-Y-D-K-V-A-E-K-F-K-E-A-F-NH₂ |
| (SEQ ID NO: 271) | Ac-E-W-L-K-A-F-Y-D-K-V-A-E-K-L-K-E-F-F-NH₂ |
| (SEQ ID NO: 272) | Ac-E-W-L-K-A-F-Y-D-K-V-F-E-K-F-K-E-A-F-NH₂ |
| (SEQ ID NO: 273) | Ac-E-W-L-K-A-F-Y-D-K-V-F-E-K-L-K-E-F-F-NH₂ |
| (SEQ ID NO: 274) | Ac-E-W-L-K-A-F-Y-D-K-V-A-E-K-F-K-E-F-F-NH₂ |
| (SEQ ID NO: 275) | Ac-E-W-L-K-A-F-Y-D-K-V-F-E-K-F-K-E-F-F-NH₂ |
| (SEQ ID NO: 276) | AC-A-F-Y-D-K-V-A-E-K-L-K-E-A-F-NH₂ |
| (SEQ ID NO: 277) | Ac-A-F-Y-D-K-V-A-E-K-F-K-E-A-F-NH₂ |
| (SEQ ID NO: 278) | Ac-A-F-Y-D-K-V-A-E-K-F-K-E-A-F-NH₂ |
| (SEQ ID NO: 279) | Ac-A-F-Y-D-K-F-F-E-K-F-K-E-F-F-NH₂ |
| (SEQ ID NO: 280) | Ac-A-F-Y-D-K-F-F-E-K-F-K-E-F-F-NH₂ |
| (SEQ ID NO: 281) | Ac-A-F-Y-D-K-V-A-E-K-F-K-E-A-F-NH₂ |
| (SEQ ID NO: 282) | Ac-A-F-Y-D-K-V-A-E-K-L-K-E-F-F-NH₂ |
| (SEQ ID NO: 283) | Ac-A-F-Y-D-K-V-F-E-K-F-K-E-A-F-NH₂ |
| (SEQ ID NO: 284) | Ac-A-F-Y-D-K-V-F-E-K-L-K-E-F-F-NH₂ |
| (SEQ ID NO: 285) | Ac-A-F-Y-D-K-V-A-E-K-F-K-E-F-F-NH₂ |
| (SEQ ID NO: 286) | Ac-K-A-F-Y-D-K-V-F-E-K-F-K-E-F-NH₂ |
| (SEQ ID NO: 287) | AC-L-F-Y-E-K-V-L-E-K-F-K-E-A-F-NH₂ |
| (SEQ ID NO: 288) | Ac-A-F-Y-D-K-V-A-E-K-F-K-E-A-F-NH₂ |
| (SEQ ID NO: 289) | Ac-A-F-Y-D-K-V-A-E-K-L-K-E-F-F-NH₂ |
| (SEQ ID NO: 290) | Ac-A-F-Y-D-K-V-F-E-K-F-K-E-A-F-NH₂ |
| (SEQ ID NO: 291) | Ac-A-F-Y-D-K-V-F-E-K-L-K-E-F-F-NH₂ |
| (SEQ ID NO: 292) | Ac-A-F-Y-D-K-V-A-E-K-F-K-E-F-F-NH₂ |
| (SEQ ID NO: 293) | Ac-A-F-Y-D-K-V-F-E-K-F-K-E-F-F-NH₂ |
| (SEQ ID NO: 294) | Ac-D-W-L-K-A-L-Y-D-K-V-A-E-K-L-K-E-A-L-NH₂ |
| (SEQ ID NO: 295) | Ac-D-W-F-K-A-F-Y-E-K-V-A-E-K-L-K-E-F-F-NH₂ |
| (SEQ ID NO: 296) | Ac-D-W-F-K-A-F-Y-E-K-F-F-E-K-F-K-E-F-F-NH₂ |
| (SEQ ID NO: 297) | Ac-E-W-L-K-A-L-Y-E-K-V-A-E-K-L-K-E-A-L-NH₂ |
| (SEQ ID NO: 298) | Ac-E-W-L-K-A-F-Y-E-K-V-A-E-K-L-K-E-A-F-NH₂ |
| (SEQ ID NO: 299) | Ac-E-W-F-K-A-F-Y-E-K-V-A-E-K-L-K-E-F-F-NH₂ |
| (SEQ ID NO: 300) | Ac-E-W-L-K-A-F-Y-E-K-V-F-E-K-F-K-E-F-F-NH₂ |
| (SEQ ID NO: 301) | Ac-E-W-L-K-A-F-Y-E-K-F-F-E-K-F-K-E-F-F-NH₂ |
| (SEQ ID NO: 302) | Ac-E-W-F-K-A-F-Y-E-K-F-F-E-K-F-K-E-F-F-NH₂ |
| (SEQ ID NO: 303) | Ac-D-F-L-K-A-W-Y-D-K-V-A-E-K-L-K-E-A-W-NH₂ |
| (SEQ ID NO: 304) | Ac-E-F-L-K-A-W-Y-E-K-V-A-E-K-L-K-E-A-W-NH₂ |
| (SEQ ID NO: 305) | Ac-D-F-W-K-A-W-Y-D-K-V-A-E-K-L-K-E-W-W-NH₂ |
| (SEQ ID NO: 306) | Ac-E-F-W-K-A-W-Y-E-K-V-A-E-K-L-K-E-W-W-NH₂ |
| (SEQ ID NO: 307) | Ac-D-K-L-K-A-F-Y-D-K-V-F-E-W-A-K-E-A-F-NH₂ |
| (SEQ ID NO: 308) | Ac-D-K-W-K-A-V-Y-D-K-F-A-E-A-F-K-E-F-L-NH₂ |
| (SEQ ID NO: 309) | Ac-E-K-L-K-A-F-Y-E-K-V-F-E-W-A-K-E-A-F-NH₂ |
| (SEQ ID NO: 310) | Ac-E-K-W-K-A-V-Y-E-K-F-A-E-A-F-K-E-F-L-NH₂ |
| (SEQ ID NO: 311) | Ac-D-W-L-K-A-F-V-D-K-F-A-E-K-F-K-E-A-Y-NH₂ |
| (SEQ ID NO: 312) | Ac-E-K-W-K-A-V-Y-E-K-F-A-E-A-F-K-E-F-L-NH₂ |
| (SEQ ID NO: 313) | Ac-D-W-L-K-A-F-V-Y-D-K-V-F-K-L-K-E-F-F-NH₂ |
| (SEQ ID NO: 314) | Ac-E-W-L-K-A-F-V-Y-E-K-V-F-K-L-K-E-F-F-NH₂ |
| (SEQ ID NO: 315) | Ac-D-W-L-R-A-F-Y-D-K-V-A-E-K-L-K-E-A-F-NH₂ |
| (SEQ ID NO: 316) | Ac-E-W-L-R-A-F-Y-E-K-V-A-E-K-L-K-E-A-F-NH₂ |
| (SEQ ID NO: 317) | Ac-D-W-L-K-A-F-Y-D-R-V-A-E-K-L-K-E-A-F-NH₂ |
| (SEQ ID NO: 318) | Ac-E-W-L-K-A-F-Y-E-R-V-A-E-K-L-K-E-A-F-NH₂ |
| (SEQ ID NO: 319) | Ac-D-W-L-K-A-F-Y-D-K-V-A-E-R-L-K-E-A-F-NH₂ |
| (SEQ ID NO: 320) | Ac-E-W-L-K-A-F-Y-E-K-V-A-E-R-L-K-E-A-F-NH₂ |
| (SEQ ID NO: 321) | Ac-D-W-L-K-A-F-Y-D-K-V-A-E-K-L-R-E-A-F-NH₂ |
| (SEQ ID NO: 322) | Ac-E-W-L-K-A-F-Y-E-K-V-A-E-K-L-R-E-A-F-NH₂ |
| (SEQ ID NO: 323) | Ac-D-W-L-K-A-F-Y-D-R-V-A-E-R-L-K-E-A-F-NH₂ |
| (SEQ ID NO: 324) | Ac-E-W-L-K-A-F-Y-E-R-V-A-E-R-L-K-E-A-F-NH₂ |
| (SEQ ID NO: 325) | Ac-D-W-L-R-A-F-Y-D-K-V-A-E-K-L-R-E-A-F-NH₂ |
| (SEQ ID NO: 326) | Ac-E-W-L-R-A-F-Y-E-K-V-A-E-K-L-R-E-A-F-NH₂ |
| (SEQ ID NO: 327) | Ac-D-W-L-R-A-F-Y-D-R-V-A-E-K-L-K-E-A-F-NH₂ |
| (SEQ ID NO: 328) | Ac-E-W-L-R-A-F-Y-E-R-V-A-E-K-L-K-E-A-F-NH₂ |
| (SEQ ID NO: 329) | Ac-D-W-L-K-A-F-Y-D-K-V-A-E-R-L-R-E-A-F-NH₂ |
| (SEQ ID NO: 330) | Ac-E-W-L-K-A-F-Y-E-K-V-A-E-R-L-R-E-A-F-NH₂ |
| (SEQ ID NO: 331) | Ac-D-W-L-R-A-F-Y-D-K-V-A-E-R-L-K-E-A-F-NH₂ |
| (SEQ ID NO: 332) | Ac-E-W-L-R-A-F-Y-E-K-V-A-E-R-L-K-E-A-F-NH₂ |

In some embodiments, an ApoA-I mimetic having the following sequence as described in U.S. Patent Application Publication No. 2006/0069030 is utilized: F-A-E-K-F-K-E-A-V-K-D-Y-F-A-K-F-W-D (SEQ ID NO: 333).

In some embodiments, an ApoA-I mimetic having the following sequence as described in U.S. Patent Application Publication No. 2009/0081293 is utilized:

(SEQ ID NO: 334)
DWFKAFYDKVAEKFKEAF;
(SEQ ID NO: 335)
DWLKAFYDKVAEKLKEAF;
(SEQ ID NO: 336)
PALEDLRQGLLPVLESFKVFLSALEEYTKKLNTQ.

In some embodiments, any of the following ApoA-I mimetics having any of the following amino acid sequences are utilized: WDRVKDLATVYVDVLKDSGRDYVSQF (SEQ ID NO: 337), LKLLDNWDSVTSTFSKLREOL (SEQ ID NO: 338), PVTOEFWDNLEKETEGLROEMS (SEQ ID NO: 339). KDLEEVKAKVQ (SEQ ID NO: 340), KDLEEVKAKVO (SEQ ID NO: 341). PYLDDFQKKWQEEMELYRQKVE (SEQ ID NO: 342), PLRAELQEGARQKLHELOEKLS (SEQ ID NO: 343), PLGEEMRDRARAHVDALRTHLA (SEQ ID NO: 344), PYSDELRQRLAARLEALKENGG (SEQ ID NO: 345), ARLAEYHAKATEHLSTLSEKAK (SEQ ID NO: 346), PALEDLROGLL (SEQ ID NO: 347), PVLESFKVSFLSALEEYTKKLN (SEQ ID NO: 348), PVLESFVSFLSALEEYTKKLN (SEQ ID NO: 349), PVLESFKVSFLSALEEYTKKLN (SEQ ID NO: 350), TVLLLTICSLEGALVRRQAKEPCV (SEQ ID NO: 351), QTVTDYGKDLME (SEQ ID NO: 352), KVKSPELOAEAKSYFEKSKE (SEQ ID NO: 353), VLTLALVAVAGARAEVSADOVATV (SEQ ID NO: 354), NNAKEAVEHLOKSELTOOLNAL (SEQ ID NO: 355), LPVLVWLSIVLEGPAPAOGTPDVSS (SEQ ID NO: 356), LPVLVVVLSIVLEGPAPAQGTPDVSS (SEQ ID NO: 357), ALDKLKEFGNTLEDKARELIS (SEQ ID NO: 358), VVALLALLASARASEAEDASLL (SEQ ID NO: 359), HLRKLRKRLLRDADDLQKRLAVYOA (SEQ ID NO: 360), AQAWGERLRARMEEMGSRTRDR (SEQ ID NO: 361), LDEVKEQVAEVRAKLEEQAQ (SEQ ID NO: 362), DWLKAFYDKVAEKLKEAF (SEQ ID NO: 363), DWLKAFYDKVAEKLKEAFPDWAKAAYDKAAEKAKEAA (SEQ ID NO: 364), PVLDLFRELLNELLEALKQKL (SEQ ID NO: 365), PVLDLFRELLNELLEALKQKLA (SEQ ID NO: 366), PVLDLFRELLNELLEALKQKLK (SEQ ID NO: 367), PVLDLFRELLNELLEALKQKLA (SEQ ID NO: 368), PVLDLFRELLNELLEALKKLLK (SEQ ID NO: 369), PVLDLFRELLNELLEALKKLLA (SEQ ID NO: 370), and PLLDLFRELLNELLEALKKLLA (SEQ ID NO: 371).

In some embodiments, amphipathic lipids include, for example, any type or combination of at least one HDL apolipoprotein component and at least one lipid component.

Examples of phospholipids which may be used in the sHDL nanoparticles include but are not limited to sphingomyelin (SM), a phosphatidylinositol, a phosphatidylserine, a phosphatidylglycerol, a phosphatidic acid, sphingosyne-1-phosphate, ceramides, lyso-phosphotydyl choline, lyso-sphingomyelin, dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), 1,2-distearoyl-sn-glycero-3-phosphatidylcholine (DSPC), dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], phosphatidylcholine, phosphatidylinositol, phosphatidylethanolamine, and combinations thereof.

In some embodiments, exemplary phospholipids include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, egg sphingomyelin, milk sphingomyelin, palmitoyl sphingomyelin, phytosphingomyelin, dipalmitoylsphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives. Phospholipid components including SM and palmitoylsphingomyelin can optionally include small quantities of any type of lipid, including but not limited to lysophospholipids, sphingomyelins other than palmitoylsphingomyelin, galactocerebroside, gangliosides, cerebrosides, glycerides, triglycerides, and cholesterol and its derivatives.

In some embodiments, the sHDL nanoparticles have a molar ratio of phospholipid/HDL apolipoprotein from 2 to 250 (e.g., 10 to 200, 20 to 100, 20 to 50, 30 to 40).

In some embodiments, the ratio of HDL apolipoprotein to phospholipid is at or between 1:1 to 1:4 wt/wt. In some embodiments, the ratio of HDL apolipoprotein to phospholipid is at or between 1:1.5 to 1:3 wt/wt. In some embodiments, the ratio of HDL apolipoprotein to phospholipid is 1:2 wt/wt. In some embodiments, the sHDL nanoparticle has less than 5% free phospholipid impurity. In some embodiments, the sHDL nanoparticle has less than 20% free HDL apolipoprotein impurity.

In some embodiments, the ratio of HDL apolipoprotein to phospholipid is at or between 1:1.5 to 1:3 wt/wt. In some embodiments, the ratio of HDL apolipoprotein to phospholipid is 1:2 wt/wt. In some embodiments, the sHDL nanoparticle has less than 5% free phospholipid impurity. In some embodiments, the sHDL nanoparticle has less than 20% free HDL apolipoprotein impurity.

In some embodiments, amphipathic lipids include, for example, any lipid molecule which has both a hydrophobic and a hydrophilic moiety.

In some embodiments, the amphipathic lipids include lipid components having a neutral phospholipid and a charged phospholipid.

As used herein, “charged phospholipids” are phospholipids that have a net charge at physiological pH. The charged phospholipid may comprise a single type of charged phospholipid, or a mixture of two or more different, typically like-charged, phospholipids. In some embodiments, the charged phospholipids are negatively charged glycerophospholipids.

The identity(ies) of the charged phospholipids(s) are not critical for success. Specific examples of suitable negatively charged phospholipids include, but are not limited to, phosphatidylgycerol, phospatidylinositol, phosphatidylserine, phosphatidylglycerol and phosphatidic acid. In some embodiments, the negatively charged phospholipid comprises one or more of phosphatidylinositol, phosphatidylserine, phosphatidylglycerol and/or phosphatidic acid.

As used herein, “neutral phospholipids” are phospholipids that have a net charge of about zero at physiological pH. In many embodiments, neutral phospholipids are zwitterions, although other types of net neutral phospholipids are known and may be used. The neutral phospholipid comprises one or both of the lecithin and/or sphingomyelin (SM), and may optionally include other neutral phospholipids. In some embodiments, the neutral phospholipid comprises lecithin, but not SM. In other embodiments, the neutral phospholipid comprises SM, but not lecithin. In still other embodiments, the neutral phospholipid comprises both lecithin and SM. All of these specific exemplary embodiments can include neutral phospholipids in addition to the lecithin and/or SM, but in many embodiments do not include such additional neutral phospholipids.

The SM may be derived from virtually any source. For example, the SM may be obtained from milk, egg or brain. SM analogues or derivatives may also be used. Non-limiting examples of useful SM analogues and derivatives include, but are not limited to, palmitoylsphingomyelin, stearoylsphingomyelin, D-erythro-N-16:0-sphingomyelin and its dihydro isomer, D-erythro-N-16:0-dihydro-sphingomyelin.

Sphingomyelins isolated from natural sources may be artificially enriched in one particular saturated or unsaturated acyl chain. For example, milk sphingomyelin (Avanti Phospholipid, Alabaster, Ala.) is characterized by long saturated acyl chains (i.e., acylchains having 20 or more carbon atoms). In contrast, egg sphingomyelin is characterized by short saturated acyl chains (i.e., acyl chains having fewer than 20 carbon atoms). For example, whereas only about 20% of milk sphingomyelin comprises C16:0 (16 carbon, saturated) acyl chains, about 80% of egg sphingomyelin comprises C16:0 acyl chains. Using solvent extraction, the composition of milk sphingomyelin can be enriched to have an acyl chain composition comparable to that of egg sphingomyelin, or vice versa.

The SM may be semi-synthetic such that it has particular acyl chains. For example, milk sphingomyelin can be first purified from milk, then one particular acyl chain, e.g., the C16:0 acyl chain, can be cleaved and replaced by another acyl chain. The SM can also be entirely synthesized, by e.g., large-scale synthesis (see, e.g., U.S. Pat. No. 5,220,043; Weis, 1999, Chem. Phys. Lipids 102(1-2):3-12).

The lengths and saturation levels of the acyl chains comprising a semi-synthetic or a synthetic SM can be selectively varied. The acyl chains can be saturated or unsaturated, and can contain from about 6 to about 24 carbon atoms. Each chain may contain the same number of carbon atoms or, alternatively each chain may contain different numbers of carbon atoms. In some embodiments, the semi-synthetic or synthetic SM comprises mixed acyl chains such that one chain is saturated and one chain is unsaturated. In such mixed acyl chain SMs, the chain lengths can be the same or different. In other embodiments, the acyl chains of the semi-synthetic or synthetic SM are either both saturated or both unsaturated. Again, the chains may contain the same or different numbers of carbon atoms. In some embodiments, both acyl chains comprising the semi-synthetic or synthetic SM are identical. In a specific embodiment, the chains correspond to the acyl chains of a naturally-occurring fatty acid, such as for example oleic, palmitic or stearic acid. In another specific embodiment, both acyl chains are saturated and contain from 6 to 24 carbon atoms.

The identity of the lecithin used is not critical for success. Also, like the SM, the lecithin can be derived or isolated from natural sources, or it can be obtained synthetically. Examples of suitable lecithins isolated from natural sources include, but are not limited to, egg phosphatidylcholine and soybean phosphatidylcholine. Additional non-limiting examples of suitable lecithins include, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, 1-palmitoyl-2-oleoylphosphatidylcholine, 1-oleoyl-2-palmitylphosphatidylcholine, dioleoylphosphatidylcholine and the ether derivatives or analogs thereof.

Lecithins derived or isolated from natural sources can be enriched to include specified acyl chains. In embodiments employing semi-synthetic or synthetic lecithins, the identity(ies) of the acyl chains can be selectively varied, as discussed above in connection with SM. In some embodiments of the charged complexes described herein, both acyl chains on the lecithin are identical. In some embodiments of charged lipoprotein complexes that include both SM and lecithin, the acyl chains of the SM and lecithin are all identical. In a specific embodiment, the acyl chains correspond to the acyl chains of myristitic, palmitic, oleic or stearic acid.

The negatively charged phospholipids can be derived from natural sources or prepared by chemical synthesis. In embodiments employing synthetic negatively charged phospholipids, the identities of the acyl chains can be selectively varied, as discussed above in connection with SM. In some embodiments of the charged lipoprotein complexes described herein, both acyl chains on the negatively charged phospholipids are identical. In some embodiments of the ternary and quaternary charged lipoprotein complexes described herein, the acyl chains on the SM, the lecithin and the negatively charged phospholipids are all identical. In a specific embodiment, the charged phospholipid(s), and/or SM all have C16:0 or C16:1 acyl chains. In another specific embodiment, the acyl chains of the charged phospholipid(s), lecithin and/or SM correspond to the acyl chain of palmitic acid. In yet another specific embodiment, the acyl chains of the charged phospholipid(s), lecithin and/or SM correspond to the acyl chain of oleic acid.

The total amount of negatively charged phospholipids(s) comprising the charged complexes can vary. Typically, the lipid component will comprise from about 0.2 to 10 wt % negatively charged phospholipids(s). In some embodiments, the lipid component comprises about 0.2 to 1 wt %, 02. to 2 wt %, 02. to 3 wt %, 0.2 to 4 wt %, 0.2 to 5 wt %, 0.2 to 6 wt %, 0.2 to 7 wt %, 0.2 to 8 wt % or 0.2 to 9 wt % total negatively charged phospholipids(s). In some embodiments, the lipid component comprises about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 wt % total negatively charged phospholipid(s), and/or a range including any of these values as endpoints. In some embodiments, the lipid component comprises from about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 wt % total negatively charged phospholipid(s) up to about 4, 5, 6, 7, 8, 9 or 10 wt % total negatively charged phospholipid(s).

It is expected that the inclusion of negatively charged phospholipids in the charged lipoprotein complexes described herein will provide the complexes with greater stability (in solution) and longer product shelf-life compared to conventional complexes. In addition, the use of negatively charged phospholipids is expected to minimize particle aggregation (e.g., by charge repulsion), thereby effectively increasing the number of available complexes present in a given dosage regime, and aid the targeting of the complex for recognition by the liver and not the kidney.

In addition to the neutral and charged phospholipids(s), the lipid component may optionally include additional lipids. Virtually any type of lipids may be used, including, but not limited to, lysophospholipids, galactocerebroside, gangliosides, cerebrosides, glycerides, triglycerides, and cholesterol and its derivatives.

In some embodiments, the lipid component further includes signaling lipids such as LPA (Lyso phoshphatidic acid): mixture of saturated (16:0, 18:0) and unsaturated (16:1, 18:1, 18:2, 20:4) species, and/or LPC (Lyso phosphatidylcholine): mixture of different species such as 16:0 (40%), 18:2 (20%), 18:1/18:0 (10-15%) and 20:4 (10%).

In some embodiments, the lipid component further includes nitrated fatty acids such as OA-NO₂ (nitrated oleic acid 9- and 10-nitro-cis-octedecenolic acids), LNO₂ (nitrated linoleic Acid 9-, 10-, 12- and 13-nitro-cis-octedecadienoic acids), AA-NO₂ (nitrated Arachidonic Acid 5-, 6-, 8-, 9-, 11-, 12-, 14,- and 15-nitro-cis-eicosatetraenoic acids), and CLNO₂ (nitrated cholesteryl linoleate cholestaryl-9-, 10-, 12- and 13-nitro-cis-octedecadiencates).

In some embodiments, the lipid component further includes fatty acids such as omega-3 polyunsaturated fatty acids including but not limited to hexadecatrienoic acid (HTA; 16:3 (n-3); all-cis-7,10,13-hexadecatrienoic acid), a-Linolenic acid (ALA; 18:3 (n-3); all-cis-9,12,15-octadecatrienoic acid), stearidonic acid (SDA; 18:4 (n-3); all-cis-6,9,12,15-octadecatetraenoic acid), eicosatrienoic acid (ETE; 20:3 (n-3); all-cis-11,14,17-eicosatrienoic acid), eicosatetraenoic acid (ETA; 20:4 (n-3); all-cis-8,11,14,17-eicosatetraenoic acid), eicosapentaenoic acid (EPA; 20:5 (n-3); all-cis-5,8,11,14,17-eicosapentaenoic acid), heneicosapentaenoic acid (HPA; 21:5 (n-3); all-cis-6,9,12,15,18-heneicosapentaenoic acid); docosapentaenoic acid (DPA; clupanodonic acid; 22:5 (n-3); all-cis-7,10,13,16,19-docosapentaenoic acid), docosahexaenoic acid (DHA; 22:6 (n-3); all-cis-4,7,10,13,16,19-docosahexaenoic acid), tetracosapentaenoic acid; 24:5 (n-3); all-cis-9,12,15,18,21-tetracosapentaenoic acid), and tetracosahexaenoic acid (Nisinic acid; 24:6 (n-3); all-cis-6,9,12,15,18,21-tetracosahexaenoic acid).

When included, such optional lipids will typically comprise less than about 50 wt % of the lipid component, although in some instances more optional lipids could be included. In some embodiments, the lipid component of the charged lipoprotein complexes does not include optional lipids.

The complexes may also optionally include other proteins, such as, for example, paraoxonase (PON) or LCAT, antioxidants, cyclodextrins and/or other materials that help trap cholesterol in the core or the surface of the complex. The complex can optionally be pegylated (e.g., covered with polyethylene glycol or other polymer) to increase circulation half-life.

The molar ratio of the lipid component to the apolipoprotein fraction of the charged lipoprotein complexes described herein can vary, and will depend upon, among other factors, the identity(ies) of the apolipoprotein comprising the apolipoprotein fraction, the identities and quantities of the charged phospholipids comprising the lipid component, and the desired size of the charged lipoprotein complex. Because the biological activity of apolipoproteins such as ApoA-I are thought to be mediated by the amphipathic helices comprising the apolipoprotein, it is convenient to express the apolipoprotein fraction of the lipid:apolipoprotein molar ratio using ApoA-I protein equivalents. It is generally accepted that ApoA-I contains 6-10 amphipathic helices, depending upon the method used to calculate the helices. Other apolipoproteins can be expressed in terms of ApoA-I equivalents based upon the number of amphipathic helices they contain. For example, ApoA-I_{M}, which typically exists as a disulfide-bridged dimer, can be expressed as 2 ApoA-I equivalents, because each molecule of ApoA-IM contains twice as many amphipathic helices as a molecule of ApoA-I. Conversely, a peptide apolipoprotein that contains a single amphipathic helix can be expressed as a 1/10-1/6 ApoA-I equivalent, because each molecule contains 1/10-1/6 as many amphipathic helices as a molecule of ApoA-I. In general, the lipid:ApoA-I equivalent molar ratio of the charged lipoprotein complexes (defined herein as “Rᵢ”) will range from about 2:1 to 100:1. In some embodiments, the Rᵢ is about 50:1. Ratios in weight can be obtained using a MW of approximately 650-800 for phospholipids.

The size of the charged lipoprotein complex can be controlled by varying the Rᵢ. That is, the smaller the Rᵢ, the smaller the disk. For example, large discoidal disks will typically have an Rᵢ in the range of about 200:1 to 100:1, whereas small discoidal disks will typically have an Rᵢ in the range of about 100:1 to 30:1.

In some specific embodiments, the charged lipoprotein complexes are large discoidal disks that contain 2-4 ApoA-I equivalents (e.g., 2-4 molecules of ApoA-I, 1-2 molecules of ApoA-I_{M} dimer or 6-10 single-helix peptide molecules), 1 molecule of charged phospholipid and 400 molecules of total neutral phospholipid. In other specific embodiments, the charged lipoprotein complexes are small discoidal disks that contain 2-4 ApoA-I equivalents, 1 molecule of charged phospholipid and 200 molecules of total neutral phospholipids.

The various apolipoprotein and/or phospholipids molecules comprising the charged lipoprotein complexes may be labeled with any art-known detectable marker, including stable isotopes (e.g., ¹¹C, ⁵N, ²H, etc.); radioactive isotopes (e.g., ¹⁴C, ³H, ¹²⁵I, etc.); fluorophores; chemiluminescers; or enzymatic markers.

In some embodiments, the lipid fraction comprises sphingosine-1-phosphate agonists, analogs, and antagonists. Examples of sphingosine-1-phosphate agonists, analogs, and antagonists include, but are not limited to, such molecules recited in U.S. Pat. Nos. 9,271,992, 9,181,331, 8,871,202, 8,802,692, 8,791,102, 8,614,103, 8,536,339, 8,524,917, 8,404,863, 8,278,324, 8,273,776, 8,263,767, 8,222,245, 8,217,027, 8,168,795, 8,143,291, 8,097,644, 8,067,549, and 8,049,037. In some embodiments, the lipid component comprises sphingosine-1-phosphate receptor agonists, sphingosine-1-phosphate receptor antagonists, and sphingosine-1-phosphate receptor analogs.

The present invention is not limited to a particular manner of generating sHDL nanoparticles.

In some embodiments, the sHDL nanoparticles encapsulate agents useful for determining the location of administered particles. Agents useful for this purpose include fluorescent tags, radionuclides and contrast agents.

Suitable imaging agents include, but are not limited to, fluorescent molecules such as those described by Molecular Probes (Handbook of fluorescent probes and research products), such as Rhodamine, fluorescein, Texas red, Acridine Orange, Alexa Fluor (various), Allophycocyanin, 7-aminoactinomycin D, BOBO-1, BODIPY (various), Calcien, Calcium Crimson, Calcium green, Calcium Orange, 6-carboxyrhodamine 6G, Cascade blue, Cascade yellow, DAPI, DiA, DID, Dil, DiO, DiR, ELF 97, Eosin, ER Tracker Blue-White, EthD-1, Ethidium bromide, Fluo-3, Fluo4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1, YOYO-3. In some embodiments, ceramides are provided as imaging agents. In some embodiments, SIP agonists are provided as imaging agents.

Additionally, radionuclides can be used as imaging agents. Suitable radionuclides include, but are not limited to radioactive species of Fe(III), Fe(II), Cu(II), Mg(II), Ca(II), and Zn(II) Indium, Gallium and Technetium. Other suitable contrast agents include metal ions generally used for chelation in paramagnetic T1-type MIR contrast agents, and include di- and tri-valent cations such as copper, chromium, iron, gadolinium, manganese, erbium, europium, dysprosium and holmium. Metal ions that can be chelated and used for radionuclide imaging, include, but are not limited to metals such as gallium, germanium, cobalt, calcium, indium, iridium, rubidium, yttrium, ruthenium, yttrium, technetium, rhenium, platinum, thallium and samarium. Additionally, metal ions known to be useful in neutron-capture radiation therapy include boron and other metals with large nuclear cross-sections. Also suitable are metal ions useful in ultrasound contrast, and X-ray contrast compositions.

Examples of other suitable contrast agents include gases or gas emitting compounds, which are radioopaque.

In some embodiments, the sHDL nanoparticles encapsulate a targeting agent. In some embodiments, targeting agents are used to assist in delivery of the sHDL nanoparticles to desired body regions. Examples of targeting agents include, but are not limited to, an antibody, receptor ligand, hormone, vitamin, and antigen, however, the present invention is not limited by the nature of the targeting agent. In some embodiments, the antibody is specific for a disease-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

In some embodiments, the sHDL nanoparticles of the present invention may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (SCIMED LIFE SYSTEMS, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

In some embodiments, the present invention also provides kits comprising sHDL nanoparticles as described herein. In some embodiments, the kits comprise one or more of the reagents and tools necessary to generate sHDL nanoparticles, and methods of using such sHDL nanoparticles.

The sHDL nanoparticles of the present invention may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the sHDL nanoparticle population and are important in the production quality control for eventual use in in vivo applications.

In some embodiments, gel permeation chromatography (GPC), which can separate sHDL nanoparticles from liposomes and free ApoA-I mimetic peptide, is used to analyze the sHDL nanoparticles. In some embodiments, the size distribution and zeta-potential is determined by dynamic light scattering (DLS) using, for example, a Malven Nanosizer instrument.

Where clinical applications are contemplated, in some embodiments of the present invention, the sHDL nanoparticles are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight sHDL nanoparticle formulation may be administered using one or more of the routes described herein.

In preferred embodiments, the sHDL nanoparticles are used in conjunction with appropriate salts and buffers to render delivery of the compositions in a stable manner to allow for uptake by target cells. Buffers also are employed when the sHDL nanoparticles are introduced into a patient. Aqueous compositions comprise an effective amount of the sHDL nanoparticles to cells dispersed in a pharmaceutically acceptable carrier or aqueous medium.

Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active sHDL nanoparticles may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active sHDL nanoparticles in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, sHDL nanoparticles are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. The sHDL nanoparticles also may be formulated as inhalants.

### EXPERIMENTAL

The following examples are provided in order to demonstrate and further illustrate certain preferred embodiments and aspects of the present invention and are not to be construed as limiting the scope thereof.

### Example 1

This example demonstrates that reconstituted HDL phospholipid compositions influence the protection against lipopolysaccharide-induced inflammation.

### Animals and Reagents

7-9 week old male and female C57BL/6 mice were purchased from Jackson Laboratories. All protocols were approved by the Institutional Animal Care & Use Committee (IACUC) at the University of Michigan, Ann Arbor.

22A (PVLDLFRELLNELLEALKQKLK (SEQ ID NO: 4)) peptide was synthesized by GenScript (Piscataway, NJ) and purity was approximately 85% as determined by HPLC. 1-palmitoyl-2-oleoyl-glycero-3phosphocholine (POPC), 1,2-dimyristoyl-sn-glycero-3-phosphocholine (DMPC), 1,2-dipalmitoyl-sn-glycero-3-phosphocholine (DPPC), and 1,2-distearoyl-sn-glycero-3-phosphocholine (DSPC) were purchased from Nippon Oil and Fat (Osaka, Japan). LPS conjugated with Alexa Fluor™ 488 was purchased from Thermo Fisher Scientific (Waltham, MA). All LPS (from E. coli 0111:B4) were purchased from Sigma Aldrich (St. Louis, MO). LPS purified by ion-exchange chromatography (L3024) was used throughout the entire experiment, except for the survival study in which LPS purified by phenol extraction (L2630) was used. Alexa Fluor 488-conjugated anti-mouse TLR4 (Clone: UT41) and Alexa Fluor 488-conjugated cholera toxin subunit B (CT-B) were obtained from Thermo Fisher Scientific (Waltham, MA). ATF-3 Antibody (C-19) (sc-188, 1:800 dilution) was purchased from Santa Cruz Biotechnology (Dallas, TX). GAPDH (D16H11) XP® Rabbit Monoclonal Antibody (5174, 1:4000 dilution) and Anti-rabbit IgG, HRP-linked Antibody (7074, 1:5000 dilution) were purchased from Cell Signaling Technologies (Danvers, MA).

### Cell Culture

RAW 264.7 and J774A.1 macrophages were cultured in Dulbecco's Modified Eagle Medium (DMEM) containing 10% fetal bovine serum (FBS), 1% Penicillin-Streptomycin (10,000 U/mL), and 100 μg/mL Normocin™. HEK-Blue™ hTLR4, which stably expresses CD14, MD2, NF-κB reporter and human TLR4, was purchased from InvivoGen (San Diego, CA) and grown in DMEM containing 10% FBS. HEK-Blue hTLR4 express the secreted embryonic alkaline phosphatase (SEAP) reporter gene under the control of the NF-κB promotor, which enables the quantification of cell activation by measuring SEAP activity in medium containing specific enzyme substrates. All cell lines were cultured at 37° C. in a humidified 5% C02 incubator.

Preparation of rHDL

rHDL were prepared via co-lyophilization procedure that were previously developed (36). Briefly, 22A (PVLDLFRELLNELLEALKQKLK (SEQ ID NO: 4)) and phosphatidylcholines (POPC, DMPC, DPPC, or DSPC) were mixed at 1:2 weight ratio in acetic acid. The resulting solution was then flash frozen in liquid nitrogen and placed on a freeze-dryer for at least 2 days to remove organic solvents. The lyophilized powder was rehydrated with phosphate buffered saline (PBS) and thermal-cycled above and below the transition temperature of each phospholipid to facilitate peptide-lipid binding. Finally, the pH of rHDL solutions was adjusted to 7.4 using NaOH and filtered with 0.2 μm sterile filter. All rHDL concentrations are expressed in terms of 22A peptide concentration.

Characterization of rHDL

The quality of the resulting rHDL was analyzed by the following analytical techniques. The purity of rHDL was determined by gel permeation chromatography (GPC), with UV detection at 220 nm, using a Tosoh TSKgel G3000SWx1 column (King of Prussia, PA). The particle size of rHDL was determined by dynamic light scattering (DLS) a on Malvern Zetasizer Nano ZSP (Westborough, MA) and the volume intensity average values were reported. The morphology of rHDL was assessed by transmission electron microscopy (TEM). rHDL samples were loaded on a carbon film-coated 400 mesh copper grid from Electron Microscopy Sciences (Hatfield, PA) that were negatively stained with 1% (w/v) uranyl formate and dried before TEM observation. All specimens were imaged with 100 kV Morgagni TEM equipped with a Gatan Orius CCD. Transition temperature (Tₘ) of rHDL were analyzed by two state modeling using TA Nano Differential Scanning Calorimetry (DSC) (New Castle, DE).

Analysis of Fluorescent-LPS Binding to rHDL

LPS conjugated with Alexa Fluor™ 488 (10 μg/mL) was pre-incubated for 1 h at 37° C., then mixed with different formulations of rHDL (1 mg/mL) and incubated for 1 hour at 37° C. Samples were centrifugated at 15000 rpm for 10 min. 25 μL of samples were injected into Shimazu Nexera-I LC 2040d Plus system connected with RF-20A prominence fluorescence detector (Kyoto, Japan) and separated with a Tosoh Bioscience TSKgel G3000W×l (7.8 mm×30 cm, 5 μm). PBS (pH 7.4) was chosen for the mobile phase with a flow rate of 0.5 mL/min. The signal was detected at 220 nm and at an excitation wavelength of 495 nm and an emission wavelength of 519 nm.

### Analysis of LPS-Induced NF-κB Expression

The HEK-Blue cell system from InvivoGen was used to analyze the neutralization of the LPS-induced inflammatory response. HEK-Blue hTLR4 cells stably express reporter-linked human TLR4, CD14, MD2, and NF-κB that are designed for studying the stimulation of human TLR4. Briefly, HEK-Blue hTLR4 cells were cultured in DMEM containing 10% low endotoxin FBS and selective antibiotics according to the manufacturer's instructions. Growth medium was discarded, and cells were resuspended in the HEK-Blue Detection medium. Cells were seeded at 25,000 cells per well. Cells were treated with various formulations of rHDL was added at a peptide concentration of 10, 30, or 100 μg/mL in a presence or an absence of 2 ng/ml of LPS. The cells were then incubated for 18 h. LPS binding to TLR4 results in the induction of NF-κB reporter expression, causing the HEK-Blue detection medium to turn blue. The blue color was quantified by measuring absorption at 650 nm using a SpectraMax M3 plate reader from Molecular Devices (San Jose, CA).

### Analysis of Pro-Inflammatory Mediators In Vitro

RAW 264.7 cells were plated in 96-well microplate at a density of 5×10⁴ cells/well and incubated until reaching 80% confluency. Cells were washed with PBS and different formulations of rHDL were added at peptide concentrations of 10, 30, or 100 μg/mL for 18 h followed by stimulation with LPS. To quantify the concentration of inflammatory cytokines including TNF-α, IL-6, and MCP-1, samples were prepared BD Cytometric Bead Array Mouse Inflammation Kit (San Jose, CA) per manufacturer's instruction. Then, prepared samples were analyzed with flow cytometry, Beckman Coulter CytoFLEX (Brea, CA).

### Cellular Cholesterol Efflux Analysis

RAW 264.7 were plated in 24-well plate at a density of 1×10⁵ cells/well and incubated for 24 h. Cells were washed with PBS once and labeled with 1 μCi of [³H] cholesterol/mL for 24 h in the growth medium. Cells were then washed with PBS and different formulations of rHDL were added at peptide concentrations of 10, 30, or 100 μg/mL in DMEM containing 0.2 mg/mL of fatty acid-free bovine serum albumin (BSA). After 18 h of incubation, media were collected, and cells lysed in 0.5 mL of 0.1% SDS and 0.1 N NaOH. Radioactive counts in media and cell fractions were measured by liquid scintillation counting using Perkin Elmer Tri-Carb 2910TR (Waltham, MA) and percent cholesterol efflux was reported by dividing the media count by the sum of the media and cell counts.

### Analysis of Lipid Raft and TLR4 Recruitment

J774A.1 were plated in 24-well microplate at a density of 5×10⁴ cells/well and incubated until reaching 80% confluency. Cells were treated with either PBS or different formulations of rHDL at a peptide concentration of 100 μg/mL for 18 h. Control group was treated with 10 mM methyl-β-cyclodextrin for 30 min. Cells were washed with ice-cold PBS containing 2% FBS. Then, cells were incubated with 8 μg/mL Alexa Fluor 594-conjugated CT-B for 15 minutes and 2 μg/mL Alexa Fluor 488-conjugated anti-TLR4 for 30 minutes to label lipid raft and TLR4, respectively. Labeled cells were washed with ice-cold PBS containing 2% FBS and the percentage distribution of lipid raft and TLR4 were reported with the mean fluorescence intensity determined by MoFlo Astrios from Beckman Coulter.

RNA isolation and RT-PCR

RAW 264.7 cells were plated in a 6-well microplate at a density of 4×10⁵ cells/well and incubated until reaching 80% confluency. Cells were then washed with PBS and different formulations of rHDL were added at peptide concentrations of 100 μg/mL for 1, 2, or 4 h. Cells were lysed and RNA was isolated using GeneJET RNA purification kit from Thermo Fisher Scientific. Approximately 1 μg of extracted RNA from each sample was transcribed to cDNA using SuperScript III First-Strand Synthesis System from Invitrogen. cDNA amplification was measured by quantitative real-time PCR on a StepOnePlus™ real-time PCR System from Applied Biosystems (Waltham, MA). TaqMan assays from Applied Biosystems were used to measure the following: Gapdh Mm99999915_gl; Atf3 Mm00476033_ml. Gene expression was determined using the ΔΔCt method using Gapdh as the housekeeping control.

### Cell Lysis and Immunoblotting:

RAW 264.7 cells were plated in 6-well microplates at 5×10⁵ cells/well and incubated until reaching 80% confluency. Cells were then washed with PBS and different formulations of rHDL were added at peptide concentrations of 100 μg/mL for 18 h. Cells were washed with ice-cold 1×PBS twice and lysed on ice for 30 min with 1×RIPA buffer (25 mM Tris-HCl pH 7.6, 150 mM NaCl, 1% NP-40, 1% sodium deoxycholate, 0.1% SDS) supplemented with cOmplete™ EDTA-free protease inhibitor cocktail and PhosSTOP from Roche (Indianapolis, IN). Lysates were clarified by centrifugation at 12,000 RPM for 10 min at 4° C. and measured protein concentration by BCA assay. An equal amount of protein per sample was loaded on 4-15% pre-casted SDS-PAGE gels from Bio-Rad (Hercules, CA) with Tris/glycine/SDS buffer and proteins were transferred onto PVDF membranes. Membranes were blocked in 5% (wt/vol) BSA in Tris-buffered saline with Tween-20 (TBS-T) for 1 h at room temperature and incubated 18 h at 4° C. with specific primary antibodies diluted in BSA. Membranes were then washed with TBS-T, incubated with secondary antibodies for 1 h, and washed with TBS-T. Images were acquired on Protein Simple FluorChem M imaging system (San Jose, CA).

Analysis of NF-κB Expression and Pro-Inflammatory Mediators from rHDL Pre-Treatment

Depending on the experiments, either HEK-Blue cells or RAW 264.7 cells were plated in 96-well microplate at a density of 25,000 or 5×10⁴ cells/well, respectively, and incubated until reaching 80% confluency. Cells were washed with PBS and different formulations of rHDL were added at peptide concentrations of 10, 30, or 100 μg/mL for 18 h. After 18 h incubation, rHDL were completely removed and cells were washed with PBS. Cells were then challenged with LPS (2 ng/mL) for 18 h again. The quantification of NF-κB expression and pro-inflammatory cytokines were obtained as described previously.

Co-Incubation of rHDL and LPS In Vivo

Female C57BL/6 mice were randomly assigned to six groups; Vehicle (PBS control group), LPS, 22A-POPC, 22A-DMPC, 22A-DPPC, and 22A-DSPC, containing five mice each. Different formulations of rHDL were pre-incubated with LPS for 30 minutes at 37° C. prior to injection. The mixtures were then administered via intraperitoneal injection (i.p.) with a final concentration of 10 mg/kg of rHDL and 0.05 mg/kg of LPS. All blood samples were collected from the jugular vein in heparinized BD centrifuge tubes (Franklin Lakes, NJ) at 2 h post-administration. Serum samples were separated immediately by centrifugation at 14,000 rpm for 10 minutes at 4° C. and stored at −80° C. until further analysis.

Effect of rHDL Infusion on the Endotoxemia Mice

Female C57BL/6 mice were randomly assigned to six groups; Vehicle, LPS, 22A-POPC, 22A-DMPC, 22A-DPPC, and 22A-DSPC, containing ten mice each. Different formulations of rHDL were administered at a dose of 10 mg/kg via intravenous injection (i.v.). Subsequently, LPS (0.05 mg/kg, i.p.) was administered. Vehicle group was dosed with PBS (i.v.) then PBS (i.p.). The LPS control group was dosed with PBS (i.v.) followed by LPS (0.05 mg/kg, i.p.). All blood samples were collected from the jugular vein in heparinized BD centrifuge tubes (Franklin Lakes, NJ) at 2 h post-LPS challenge. Serum samples were separated immediately by centrifugation at 14,000 rpm for 10 minutes at 4° C. and stored at −80° C. until further analysis.

### In Vivo Analysis of Pro-Inflammatory Mediators

The concentrations of inflammatory mediators, TNF-α, IL-6, and MCP-1 in the serum of LPS co-incubation study were quantified using eBioscience Ready-Set-Go ELISA (San Diego, CA) per manufacturer's instruction. The concentration of inflammatory mediators of TNF-α, IL-6, and MCP-1 in the serum of endotoxemia model was quantified using BD Cytometric Bead Array Mouse Inflammation Kit per manufacturer's instruction and analyzed. on Beckman Coulter CytoFLEX Flow Cytometer.

### Survival Determination

Male C57BL/6 were randomly assigned into three groups, containing ten mice each; Vehicle, LPS, and 22A-DMPC. The vehicle group received PBS (i.p. and i.v.). The LPS group was first received LPS (10 mg/kg, i.p.). Once the anal temperature increased 0.5° C. from LPS (approximately 15 min), PBS (i.v.) was administered. The 22A-DMPC group was first received LPS (10 mg/kg, i.p.). Again, once the anal temperature increased 0.5° C. from LPS (approximately 15 min), 22A-DMPC (10 mg/kg, i.v.) was administered. The mice were then observed for mortality every 6 h and survival rates were recorded. Their lungs and livers were isolated collected for histological evaluation.

### Tissue Preparation

Tissues were fixed in 10% neutral buffered formalin for a minimum of 24 h. Histology preparation was performed by the Unit for Laboratory Animal Medicine In Vivo Animal Core at the University of Michigan. Briefly, tissues were cassetted and processed to paraffin on an automated processor, TissueTek VIP 6 from Sakura (Torrance, CA). Tissues were embedded in paraffin, sectioned at 4 μm thickness on a rotary microtome, and mounted on glass slides. Slides were stained with hematoxylin and eosin on an automated histostainer and coverslipped.

### Histology Evaluation and Images

Histological sections were evaluated using light microscopy at magnifications ranging from 20× to 600× by a board-certified veterinary pathologist using an Olympus BX45 light microscope (Tokyo, Japan) Corporation). The evaluation was performed without knowledge of the experimental groups. Representative images were taken after histology analysis using an Olympus DP73 microscope-mounted camera with associated software, Olympus cellSens v 1.18 (Tokyo, Japan). Images were processed into figures using Adobe Photoshop CC v 19.0. Image processing was confined to global adjustments of white balance, brightness, contrast, and sharpness that did not affect image interpretation. Histology was assessed based on standardized nomenclature/criteria for rodent hepatobiliary lesions (37) and literature descriptions of relevant histology in LPS challenge experiments (38-41).

### Statistical Analysis

Statistical differences were compared with Student's t-test for comparing two groups or with one-way analysis of variance (ANOVA) with Tuckey's post-hoc test for comparing multiple groups. All samples were performed in triplicate unless noted otherwise. P<0.05 was considered statistically significant. The Chi-square test was used to compare survival rates. Statistical analysis was performed using GraphPad Prism 7 (La Jolla, CA). Measurements are presented as means f standard error of the mean unless indicated otherwise.

### Cell Viability Analysis

RAW 264.7, J774A.1, and HEK-Blue™ hTLR4 cells were plated in 96-well microplates at a density of 5×10′ cells/well and incubated until reaching 80% confluency. Cells were washed with PBS and incubated with various formulations of rHDL at a concentration of 100 μg/mL for 18 h. Cells were washed with PBS and cell viability was assessed using Promega CellTiter 96® AQueous One Solution Cell Proliferation Assay (Madison, WI) per manufacturer's instruction.

### Lethal-Endotoxemia Model Optimization

Male C57BL/6 were randomly assigned into three groups, containing ten mice each, to determine the appropriate concentration of LPS for inducing lethal endotoxemia via a single i.p. injection of LPS (5 mg/kg, 10 mg/kg, and 20 mg/kg). The mice were then observed for mortality every 6 h for 4 days, and survival rates were recorded.

### 22A-DSPC Treatment Survival Determination

Male C57BL/6 were randomly assigned into three groups, containing ten mice each; Vehicle, LPS, and 22A-DSPC. The vehicle group received PBS (i.p. and i.v.). The LPS group was first received LPS (10 mg/kg, i.p.). Once the anal temperature increased 0.5° C. from LPS (approximately 15 min), PBS (i.v.) was administered. The 22A-DSPC group was first received LPS (10 mg/kg, i.p.). Again, once the anal temperature increased 0.5° C. from LPS (approximately 15 min), 22A-DSPC (10 mg/kg, i.v.) was administered. The mice were then observed for mortality every 6 h and survival rates were recorded. Their lungs and livers were collected for histological evaluation.

Preparation and Characterization of rHDL

rHDL were prepared by complexing apoA-I mimetic peptide, 22A, with various PCs (POPC, DMPC, DPPC, or DSPC) using a co-lyophilization procedure. Based on preliminary studies, the optimal weight ratio of peptide to phospholipid to result in homogenous prep-like HDL is at 1:2 wt/wt peptide to phospholipid (42). To validate the morphology and confirm prep-like discoidal shape, each rHDL formulation was observed with TEM (FIG. 1A). 22A-DMPC, 22A-DPPC, and 22A-DSPC were observed with typical discoidal morphology and were uniform in size. In contrast, 22A-POPC displayed heterogeneity in both size distribution and morphology. This is plausibly owing to presence of liposomal impurities. These characteristics were further confirmed with DLS. We (the inventors of the current patent application) observed that the average diameters for 22A-POPC (13.7±0.2 nm), 22A-DMPC (9.7±0.2 nm), 22A-DPPC (11.2±0.3 nm), and 22A-DSPC (12.2±0.3 nm) were all within range of previously reported rHDL sizes (42, 43) (Table 3). Analysis via GPC verified the observed size differences and the purity of rHDL (FIG. 1C). All rHDL formulations resulted in a similar retention time of approximately 8 min (Table 3), with 22A-POPC exhibiting a broader peak, indicating a more heterogeneous size distribution. The small peaks appearing at approximately 11 min represent free 22A peptide, accounting for less than 2% and considered negligible for all formulations.

TABLE 3
Characterization Summary of rHDL. The particle size
of rHDL nanoparticles was analyzed by DLS; RT and purity were
analyzed by GPC; and Tm of rHDL was analyzed by DSC (mean ± STD).

| rHDL | RT | Particle Size | Purity | rHDL Tm |
|---|---|---|---|---|
| Formulation | (min) | (nm) | (%) | (° C.) |
| 22A-POPC | 7.9 | 13.7 ± 0.2 | 99.3 ± 0.1 | 0.5 ± 0.5 |
| 22A-DMPC | 8.2 | 9.7 ± 0.2 | 99.2 ± 0.1 | 27.0 ± 0.0 |
| 22A-DPPC | 7.8 | 11.2 ± 0.3 | 98.0 ± 0.8 | 45.4 ± 0.4 |
| 22A-DSPC | 7.7 | 12.2 ± 0.3 | 99.2 ± 0.0 | 57.8 ± 1.3 |

RT: Retention Time,
Tm: transition temperature

Next, the Tₘ of each rHDL formulation was evaluated, as shown in Table 3. POPC is composed of 16:0/18:1 fatty acids, in which the unsaturated fatty acid causes a significantly low Tₘ (−3.3±0.5° C.) (44). When complexed with 22A, 22A-POPC had an observed Tₘ value of 0.5±0.5° C. Similarly, DMPC is composed of 14:0/14:0 (Tₘ: 24.5° C.) (45), DPPC is composed of 16:0/16:0 (Tₘ: 41.6° C.) (45), and DSPC is composed of 18:0/18:0 (Tₘ: 54.5° C.) (45). Once complexed with 22A to form rHDL, we observed rHDL Tₘ values of 27.0 f 0.0° C., 45.4±0.4° C., and 57.8±1.3° C., respectively. A slight temperature rise from PC to rHDL is observed, possibly due to the addition of 22A peptide adding rigidity to the phospholipids. Based on Tₘ of each rHDL, 22A-POPC and 22A-DMPC are preferentially at fluid and mobile liquid crystalline phase, while 22A-DPPC and 22A-DSPC are at rigid and constrained gel phase at physiological temperature (37° C.).

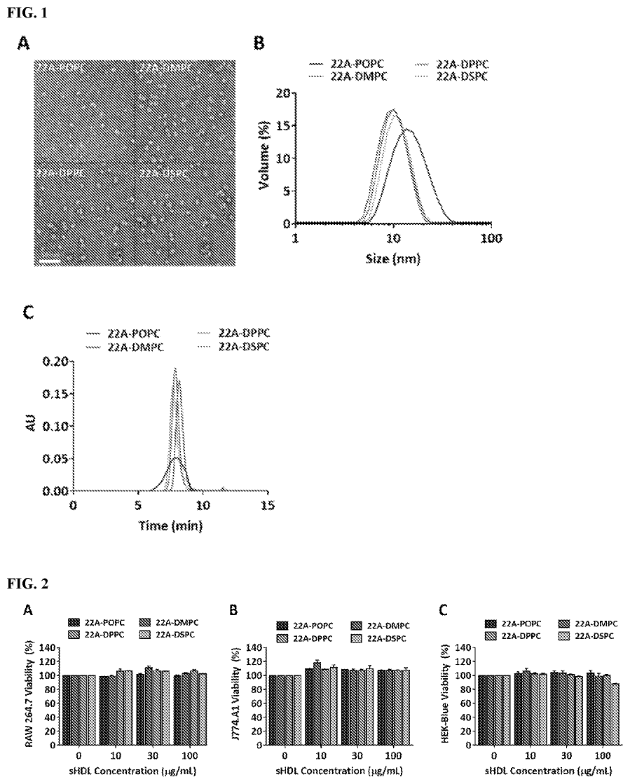

We further assessed the cytotoxicity of rHDL in multiple cell lines and none of the formulations exhibited cytotoxicity at concentrations up to 100 μg/mL (FIG. 2). Taken together, our results indicate the successful production of non-cytotoxic rHDL with prep-like morphology and size.

rHDL Binding and Neutralization of LPS

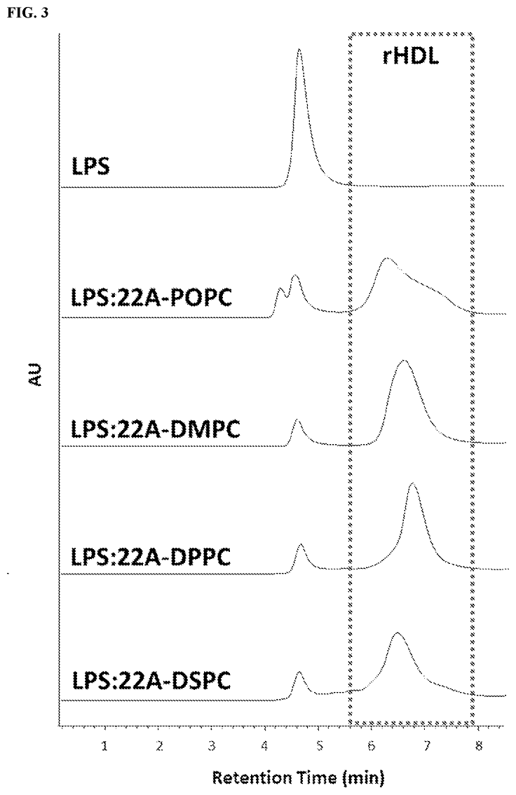

HDL can directly neutralize the TLR4-mediated inflammatory cascade by sequestering LPS in its phospholipid layer (29-31). To investigate whether rHDL made from different phospholipids could successfully neutralize LPS, a TLR4 ligand, by direct interaction, we analyzed the size-exclusion profile of rHDL after incubation with fluorescent LPS. As expected, all formulations of rHDL promoted a shift of LPS-Alexa 488 to rHDL molecular weight fraction, demonstrating successful binding of LPS to rHDL. (FIG. 3).

Effect of rHDL Lipids Against LPS-Induced Inflammation In Vitro

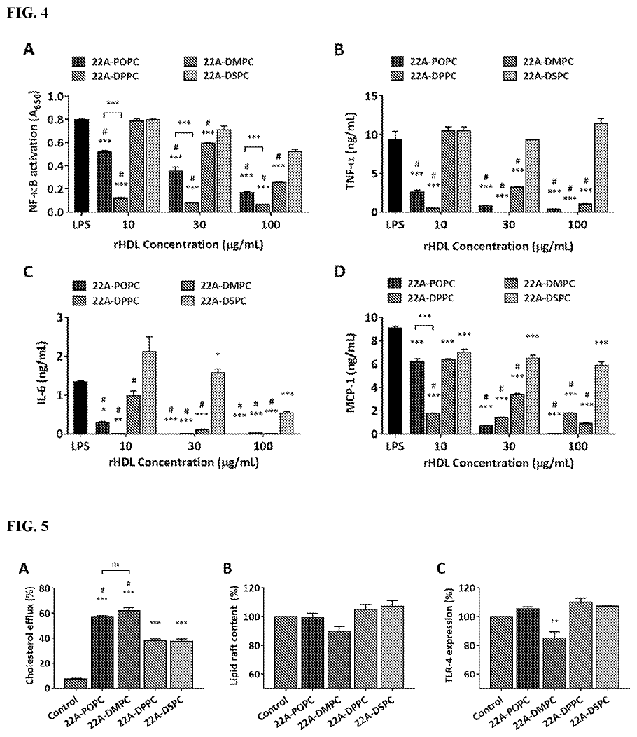

We next sought to evaluate how ability of rHDL to sequester LPS could be translated to inhibition of inflammatory response, as TLR4 mediated recognition of LPS is thought to be one of the key triggers of the inflammatory response (3, 4). To understand which formulations of rHDL most effectively modulate TLR4-mediated signaling, the HEK-Blue cell system was used to quantify the activity of NF-κB. HEK-blue hTLR4 cells were incubated with different rHDL at various concentrations (10, 30, and 100 μg/mL) in the presence of LPS (2 ng/mL). 22A-POPC and 22A-DMPC displayed significant concentration-dependent inhibition of NF-κB (P<0.001) and inhibited NF-κB at all tested concentrations, 22A-DPPC inhibited activity at concentrations of 30 μg/mL and greater, and 22A-DSPC had no effect (FIG. 4A). When comparing the two most potent inhibitors, 22A-POPC and 22A-DMPC, 22A-DMPC showed enhanced inhibition at all concentrations (P<0.001).

We further examined the downstream TLR4-mediated inflammatory response by evaluating pro-inflammatory cytokine production. To do this, macrophages were incubated with rHDL at various concentrations in the presence or absence of LPS (2 ng/mL). Concentrations of TNF-α, IL-6, and MCP-1 in the media were quantified. 22A-POPC, 22A-DMPC, and 22A-DPPC effectively reduced LPS-induced pro-inflammatory mediators compared to controls (P<0.001) and to 22A-DSPC (#P<0.05) (FIG. 3 B-D). Here, we observed that fluid liquid crystalline phase 22A-DMPC resulted in the greatest inhibition of NF-κB activity and pro-inflammatory cytokine production. 22A-DPPC, although in the rigid gel phase, decreased the inflammatory response at the highest concentration as Tₘ is near physiological temperature, while 22-DSPC showed the least effective in LPS-induced inflammatory response modulation due to its limited fluidity making incorporating LPS into its phospholipid layer difficult.

Effect of rHDL on TLR4 Recruitment into Lipid Raft Via Cholesterol Efflux

Lipid raft plays an important role for LPS-induced cellular activation. HDL promotes cholesterol efflux from macrophages via reverse cholesterol transport, compromising the integrity of lipid rafts as cholesterol is depleted leading to reduced lipid raft and TLR4 recruitment into lipid raft (8). First, to demonstrate whether rHDL could efflux cholesterol from macrophages, we incubated different rHDL (100 μg/mL) with [³H]-cholesterol-loaded macrophages. 22A-DMPC (61.8±2.7%) exhibited the greatest cholesterol efflux, followed by 22A-POPC (57.2±0.9%), 22A-DPPC (38.1±1.4%), and 22A-DSPC (37.5±1.9%) (FIG. 5A). Given these results, we then examined the impact of rHDL-mediated cholesterol efflux on the alteration of lipid raft and TLR4 surface expression. Intriguingly, despite the significant cholesterol efflux observed from all rHDLs, none markedly reduced the lipid raft content, where only 22A-DMPC non-significantly reduced the lipid raft (90.1 f 3.1%) (FIG. 5B). However, minimal reduction of lipid rafts of 22A-DMPC was capable of significant decrease of TLR4 recruitment on the cell surface (85.1±4.5%) (FIG. 5C). Overall, these results indicate that only 22A-DMPC was capable of inhibiting TLR4 recruitment into lipid raft via cellular membrane cholesterol depletion.

Effect of rHDL on ATF3 Expression

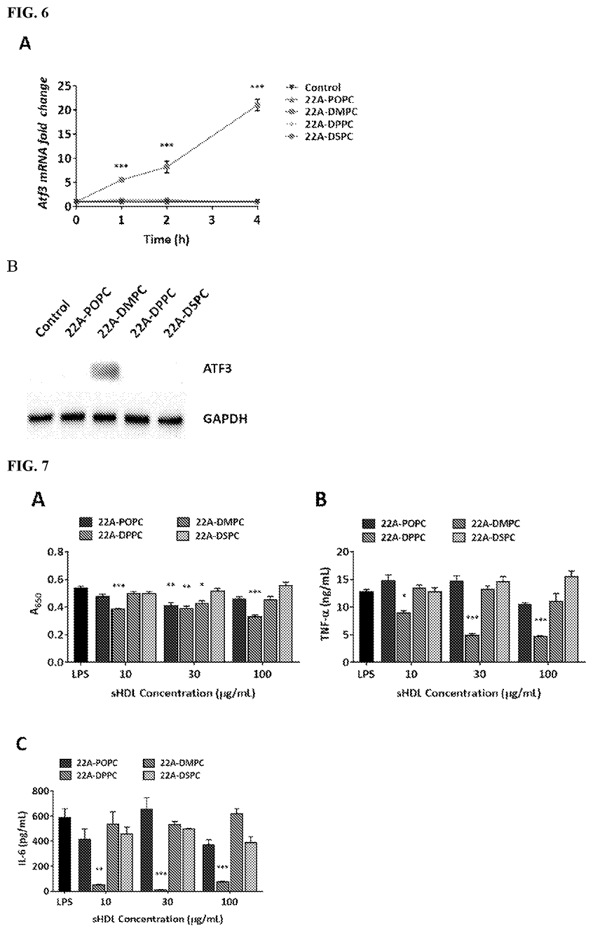

ATF3 is a negative regulator of macrophage activation, acting as a negative-feedback system upon TLR4 activation to limit excess production of pro-inflammatory cytokines (46, 47). A few studies have shown that HDL can regulate the expression of TLR-induced pro-inflammatory cytokines on the transcriptional level via the transcriptional repressor ATF3 (9-11). To examine the ability of rHDL to promote ATF3 expression, we incubated macrophages with rHDL (100 μg/mL) and determined the mRNA and protein expression of ATF3. Notably, only 22A-DMPC induced Atf3 mRNA expression significantly, increasing 5-fold in the first 1 h up to 21-fold after 4 h of incubation (P<0.001), while 22A-POPC, 22A-DPPC, or 22A-DSPC had no effect (FIG. 6A). ATF3 protein expression was also examined by incubating macrophages with different rHDL for 18 h. Likewise, only 22A-DMPC induced prominent protein expression of ATF3, whereas expression was undetectable for the control group and other rHDL formulations (FIG. 6B).

Pre-Treatment of rHDL Against LPS-Induced Inflammation In Vitro

Previous assessments revealed that among the different formulations of rHDL, only 22A-DMPC reduced TLR4 recruitment and promoted ATF3 expression. Here, we further demonstrated how these mechanisms can be elucidated to modulate the inflammatory response. Macrophages were incubated with different rHDL at various concentrations (10, 30, and 100 μg/mL). After 18 h incubation, rHDL were completely removed then cells were challenged with LPS (2 ng/mL) for 18 h again. When NF-κB expression was quantified from HEK-Blue hTLR4 cells, only 22A-DMPC showed significant inhibition in NF-κB expression in a dosage-dependent manner (P<0.001) (FIG. 7A). Similarly, when pro-inflammatory cytokines were measured from macrophages, again, only 22A-DMPC significantly altered TNF-α and IL-6 levels (P<0.001) (FIG. 7B-C). This study confirms that rHDL can promote anti-inflammatory activities not only through physical binding to LPS, but also through compromising lipid raft integrity to decrease TLR4 expression and promote ATF3 expression.

Effect of rHDL on LPS Neutralization In Vivo

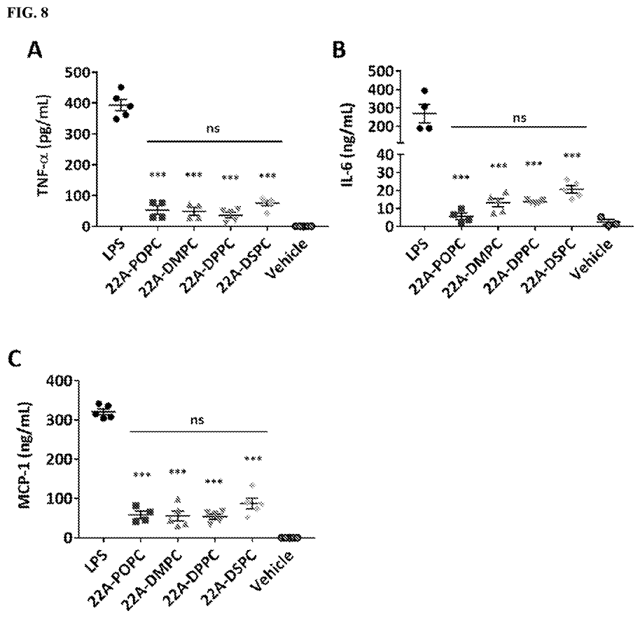

We examined whether LPS neutralization by rHDL effectively translates to modulation of the inflammatory response in vivo by administering pre-mixed LPS and rHDL solutions. Briefly, each rHDL were incubated with LPS for 30 min at 37° C. and the mixture of each rHDL (10 mg/kg) and LPS (0.05 mg/kg) was administered to mice. When pro-inflammatory mediators including TNF-α, IL-6, and MCP-1 were analyzed at 2 h post-administration, all formulations of rHDL surprisingly suppressed their secretion (P<0.001), however, no statistical differences were identified between the different rHDL formulations (FIG. 8). This study strongly suggests that rHDL can effectively neutralize LPS-induced inflammatory activity by direct interaction.

Effect of rHDL on LPS-Induced Endotoxemia Mice

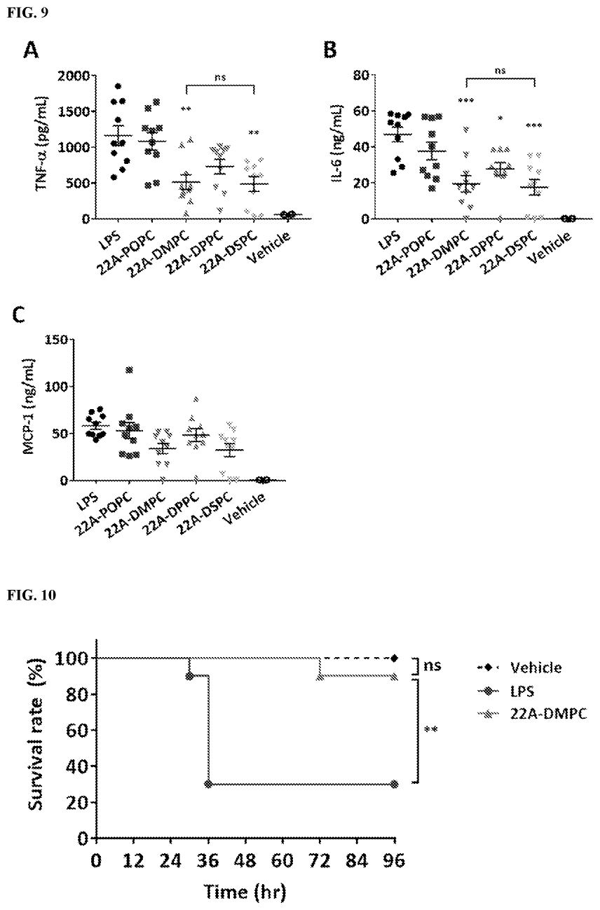

The ability of rHDL to elicit anti-inflammatory effect in vivo was examined in a murine endotoxemia model. Mice were initially administered with LPS (0.05 mg/kg) followed by different formulations of rHDL (10 mg/kg). 2 h post-LPS challenge, 22A-DMPC and 22A-DSPC caused a significant inhibition of TNF-α and IL-6 (P<0.01 and P<0.001, respectively), while 22A-DPPC resulted in a slight reduction of IL-6 (P<0.05) and 22A-POPC had no effect (FIG. 9A-B). In addition, 22A-DMPC and 22A-DSPC attenuated levels of MCP-1, although they were not statistically significant (FIG. 9C). This study indicated that 22A-DMPC and 22A-DSPC were the only formulations able to effectively attenuate the inflammatory response in endotoxemia mice.

Effect of rHDL on Lethal Endotoxemia and Organ Injury

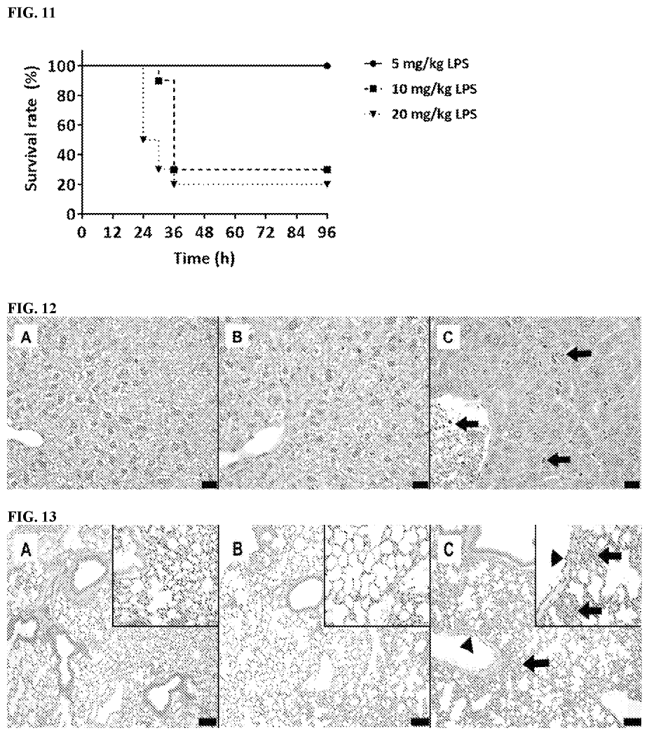

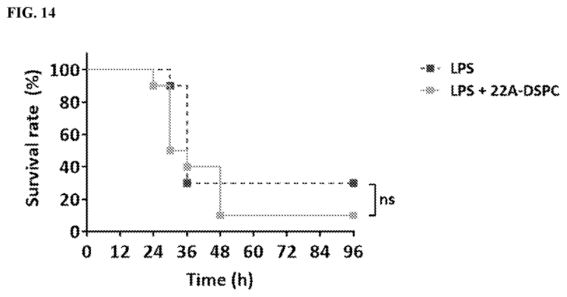

We further hypothesized that rHDL, especially 22A-DMPC, could improve the survival rate and protect organ injury from lethal endotoxemia, as it was observed to promote exceptional anti-inflammatory activities in vitro and in mild endotoxemia mice. Once we determined the appropriate concentration of LPS for lethal endotoxemia (FIG. 11), mice were administered with a lethal endotoxin concentration of LPS (10 mg/kg). After the anal temperature had risen 0.5° c., mice were administered 22A-DMPC (10 mg/kg), and their survival was monitored for 4 days. As shown in FIG. 9 and Table 4, the survival in the 22A-DMPC treatment group drastically improved from 30% to 90% compare to the LPS-only group (P<0.01). Additionally, the mean survival time in the 22A-DMPC treatment group prolonged dramatically from 53.4±9.3 h to 93.6±2.4 h relative to LPS-only treated animals (P<0.001). There were no statistical differences observed between the 22A-DMPC group and the vehicle group.

TABLE 4
The treatment effect of 22A-DMPC rHDL
in lethal endotoxemia mice.

| Group (n = 10) | Survival rate (%) | Survival time (h) |
|---|---|---|
| LPS | 30** | 53.4 ± 9.3*** |
| 22A-DMPC | 90** | 93.6 ± 2.4*** |

**P > 0.01,
***P > 0.001

We further compared the relative severity of LPS-induced pulmonary and liver pathology in the rHDL treatment group and LPS group. As shown in FIG. 12 (liver), FIG. 13 (lung), and Table 5, the 22A-DMPC treatment group showed no alterations, no inflammatory infiltrations, and displayed similar pathology to the negative control group. In contrast, positive LPS controls had the greatest histological changes in both liver and lung, consisting of inflammatory cell infiltration in the liver and lung, hepatocyte degeneration, hepatic glycogen depletion, and hepatic extramedullary hematopoiesis of the myeloid lineage. These findings evidently indicate that rHDL, particularly 22A-DMPC, could protect endotoxemia mice from death and organ injury.

TABLE 5
Histology evaluation.

| | | Severity scores of | | |
|---|---|---|---|---|
| | | representative animals | | |
| | | | 22A- | |
| Tissue | Finding | Control | DMPC | LPS |
| Liver | Infiltration, neutrophilic and | 0 | 0 | 1 |
| | macrophagic, sinusoidal | | | |
| | Glycogen depletion | 0 | 0 | 3 |
| | Infiltration, neutrophilic and | | | |
| | Lung | 0 | 1 | 2 |
| | macrophagic, interstitial and | | | |
| | perivascular | | | |

Severity scores: 0 (not present);
1 (mild: few small foci or minimal density);
2 (moderate: multiple small foci or moderate density);
3 (severe: multiple larger or coalescing foci or regionally intense)

### DISCUSSION

In the present study, we extensively investigated how changes of phospholipids in rHDL can impact the anti-inflammatory activities in LPS-induced inflammation. We used PC that varies in fatty acid chain length and saturation in the synthesis of rHDL, to display a unique fluidity of rHDL. The fluidity of the rHDL is known to be increased with the degree of the unsaturated fatty acid moieties (30). To this extent, HDL containing polyunsaturated saturated fatty acids (PUFA) would exhibit more fluid PC phase and result in enhanced anti-inflammatory activities by accelerating efflux of cell-derived pro-inflammatory lipids, LPS, and cholesterol. However, our studies demonstrated that the rHDL with Tₘ closer to physiological temperature, 22A-DMPC resulted in the most enhanced anti-inflammatory activity in various mechanisms including LPS neutralization, cholesterol efflux, reduced TLR4 recruitment, and induced ATF3 expression to promote the greatest anti-inflammatory from fluid and mobile 22A-DMPC.

We first analyzed NF-κB, pro-inflammatory mediators quantify the capability of each rHDL to neutralize LPS and modulate inflammatory signaling cascade. 22A-DMPC notably suppressed NF-kB expression and pro-inflammatory mediators followed by 22A-POPC, 22A-DPPC, and 22A-DSPC (FIG. 4). According to PUFA residues (35, 48), 22A-POPC exhibit the greatest fluidity and expected to result in the greatest LPS neutralization, nevertheless, 22A-DMPC led to greatest LPS neutralization. When 22A-DMPC, 22A-DPPC, and 22A-DSPC are compared, the LPS neutralization capability was correlated to the fatty acid chain length of PC as it affected the fluidity of rHDL. However, LPS neutralization capability between 22A-POPC and 22A-DMPC did not follow the degree of the fluidity and the difference could be potentially explained by the stability of rHDL. We believe that heterogenous size distribution in 22A-POPC is possibly due to a presence of liposomal impurities. The difficulty of synthesizing pure 22A-POPC was described previously and was attributed to fluidity and instability of phospholipid membrane at room temperature which is above POPC Tₘ (49). In addition, the presence of unsaturated fatty acid chain could result in phospholipid oxidation and oxidized HDL has been reported with less fluidity (50, 51). The instability and reduced fluidity of 22A-POPC would have caused reduced LPS neutralization capability. 22A-DMPC, in contrast, exhibits fluid liquid crystalline phase yet is relatively stable at physiological temperature since its Tₘ was close to physiological temperature. Biophysical characterizations should be further investigated to validate the fluidity of each rHDL through changes in fluorescence polarization anisotropy and how the fluidity influences the interaction with LPS.

Next, we sought to explore the disruption of lipid raft integrity, thereby decreasing TLR4 on the surface of the cell as TLR4 presentation is localized within lipid rafts. Numerous studies have reported differences in cholesterol efflux capability for PCs of different saturation and fatty acid chain length. For example, saturated long-chain phospholipids such as DPPC and DSPC have higher cholesterol efflux capabilities and higher physical binding affinity to cholesterols than POPC (32, 52-54). Our result was marginally in discordance with previous reports, as we focused on the fluidity of rHDL rather than physical cholesterol binding affinity. Analogous to our LPS binding results, we observed the greatest cholesterol efflux capacity from 22A-DMPC followed by 22A-POPC, and the least capacity from 22A-DPPC and 22A-DSPC based on its fluidity to efflux cholesterol.

Despite the significant cholesterol depletion observed with 22A-DMPC, it did not notably reduce the lipid raft content. Nevertheless, the modest lipid raft reduction was sufficient to result in a significant decrease of TLR4 recruitment on the cell surface (FIG. 5). Murphy et al. performed a similar study examining the change in human monocyte lipid raft content, however, they observed a dramatic reduction of lipid raft content with apoA-I treatment (55). Several past studies reported that reduction of lipid raft cholesterol occurs through ABCA1 which is a key transporter that primarily interacts with apoA-I to efflux cholesterol from macrophages (8, 56). Therefore, one explanation for why the high cholesterol efflux from 22A-DMPC did not translate to a dramatic decrease in lipid rafts is that 22A-DMPC was initiating cholesterol efflux through another transporter, such as ATP-binding cassette transporter G1 (ABCG1), scavenger receptor B type I (SR-BI), or by passive diffusion. ABCG1 transporters interact with mature HDL to efflux cholesterol while SR-BI transporter is located in the caveolar region of macrophage and promotes bidirectional cholesterol efflux. Validation of 22A-DMPC or other rHDL transporter-specific efflux in macrophages would explain the relative minimal lipid raft disruption compared to the dramatic observed cholesterol efflux capacity.

We also showed stimulation of ATF3 expression by rHDL, consistent with initial reports by De Nardo et al. (9), and further demonstrated that expression of ATF3 is critically dependent on rHDL phospholipid composition. Activation of ATF3 leads to recruitment of histone deacetylase 1 to the promoter region of pro-inflammatory cytokine gene and assists in deacetylating to limit transcriptional binding (57, 58). In addition, a recent study demonstrated that ATF3 can directly interact with the p65 subunit of NF-κB to attenuate the NF-κB activity, thus, modulate the inflammatory response, rather than via indirect histone deacetylase 1 pathway (59). We demonstrated that 22A-DMPC induced prominent expression of ATF3 mRNA and protein while other rHDLs had no effect (FIG. 6). Several studies also explored how structural difference in HDL can impact ATF3 expression. Didchenko et al. confirmed the finding that large rHDL have reduced ability to induce ATF3 and smaller rHDL have strong ability to induce both anti-inflammatory phenotypes and ATF-3 expression in macrophages (60). Similarly, Wang et al. found that various apoA-I cysteine mutants of rHDL can cause altered ATF3 expression, as amino acid mutations can influence the ATF3 pathway in macrophages (10). The mechanism by which HDL activates ATF3 is still unclear, but it is well-recognized as a key inflammatory modulator of HDL. We propose that rHDL phospholipid composition and fluidity of rHDL can also influence the ATF3 pathway in macrophages causing altered expression of ATF3.

Interestingly, we were surprised by the results from our in vivo endotoxemia model (0.05 mg/kg LPS) with rHDL administration (10 mg/kg). While we expected to see enhanced suppression of inflammatory response from the 22A-DMPC group due to its promising results in vitro, we did not expect that the 22A-DSPC group would also show a prominent suppression of the inflammatory response (FIG. 9). In vitro 22A-DSPC was the least effective among all rHDL treatments, and we believe that 22A-DSPC efficacy in endotoxemia model owes to its exceptional half-life. The half-life of 22A-DSPC was nearly 2-fold longer than 22A-DMPC, observed from a pharmacokinetic study (in revision, JPET), which may have allowed for greater exposure and neutralization of LPS (61). A credible explanation is that rHDL is known to be dissociated and remodeled upon administration in vivo (43, 62), thus, 22A-DSPC favorably remodeled at a slower rate due to its rigid gel phase phospholipid compared to the fluid liquid crystalline phase phospholipid of 22A-DMPC (in revision, JPET). Nevertheless, the prolonged circulation of 22A-DSPC did not translate to a decrease in LPS-induced inflammation or an increase in survival following lethal doses of LPS (FIG. 14). Conversely, the efficacy of 22A-DMPC was further demonstrated in a lethal endotoxemia model which increased the survival rate and time (Table 4) along with the prevention of inflammation-induced organ injury (Table 5).

In 1993, Levine et al. reported that rHDL (80 mg/kg) composed with 18A peptide and egg PC could improve the survival rate 3- to 4-fold in endotoxemia mice (10 mg/kg LPS) and suggested the simple leaflet insertion model for neutralization of LPS by phospholipid on the surface of HDL (31). Imai et al. found that administration of apoA-I (10 mg/kg) 2 h post-LPS injection (1 mg/kg LPS) reduce plasma TNF-α and increased the survival rate in endotoxemia rats (5 mg/kg). Similarly, Yan et al. demonstrated the administration of apoA-I (100 mg/kg) to endotoxemia mice (5 mg/kg LPS) significantly lowered mortality (26). Zhang et al. used rHDL (40 mg/kg) complexed with apoA-I Milano, a mutant apoA-I, with soy PC as a treatment in endotoxemia rats (400 EU/kg gram-negative bacteria endotoxin) and observed improvements in renal and hepatic functions as well as a reduction in pro-inflammatory cytokines (27). In addition, Wang et al. compared the anti-inflammatory effect of rHDL containing different apoA-I cysteine mutants in endotoxemia mice, suggesting the cysteine mutation can impact LPS neutralization capability (63). The results from our study are consistent with these previous findings, with rHDL exerting efficacy against LPS-induce endotoxemia by inhibiting pro-inflammatory mediators and improving the survival rate in endotoxemic mice. Moreover, we proposed the importance of rHDL phospholipid composition which influences the mechanisms of anti-inflammatory activities in LPS-induced inflammation.

In conclusion, for the first time, we demonstrate that phospholipid composition drastically alters the anti-inflammatory effect of rHDL on LPS-induced inflammation both in vitro and in vivo. Our data suggest that fluidity of rHDL due to structural variances of phospholipids critically determines the anti-inflammatory effect by promoting different anti-inflammatory mechanisms. In this study, 22A-DMPC exhibited the most fluid yet stable rHDL at physiological temperature, displaying greatest anti-inflammatory activities through multiple mechanisms including LPS neutralization, disruption of lipid raft integrity, and activation of ATF3 in vitro but also protected mice against mortality and organ injury from lethal endotoxemia. Therefore, we suggest that 22A-DMPC may be a potential therapeutic effect against LPS-induced sepsis.

### Example II

### HDL Levels Drop in COVID-19 and Other Infectious Diseases.

Based on the epidemiological data from China, COVID-19 mortality is the highest in patients with underlying cardiovascular disease and diabetes. These patients already have underlying endothelial dysfunction and dysregulation of lipid metabolism, which is likely contribute to increase mortality. Lowering of serum lipid levels, especially total cholesterol (TC) and HDL cholesterol (HDL-C), have been reported to occur in during human immunodeficiency virus (HIV) and hepatitis C virus (HCV) infections. It has been reported that low serum cholesterol levels among patients with COVID-19 infection in Wenzhou, China. In these patients the reported levels of TC and HDL-C(3.70±0.02 and 1.18±0.03 mmol/L) were sharply decreased relative to the age and sex matched controls (4.91±0.10 and 1.47±0.03 mmol/L, p<0.001). The daily HDL-C measurements indicated persistent drop until the 9^{th} day of infection and slow recovery as infection subsided (FIG. 16).

Endogenous HDL offers vascular protection during infection by reducing pro-inflammatory cytokine release from the immune effector cells, inhibiting endothelial activation and scavenging lipid oxidative species. It has been shown previously that the HDL-C levels are markedly reduced in septic patients with pneumonia, with levels on average 45% lower compared to non-septic controls. Furthermore, HDL-C levels on the first day in ICU are predictive of overall patient survival.

Infusion of sHDL Offers Protection in Sepsis by Multiple Mechanisms.

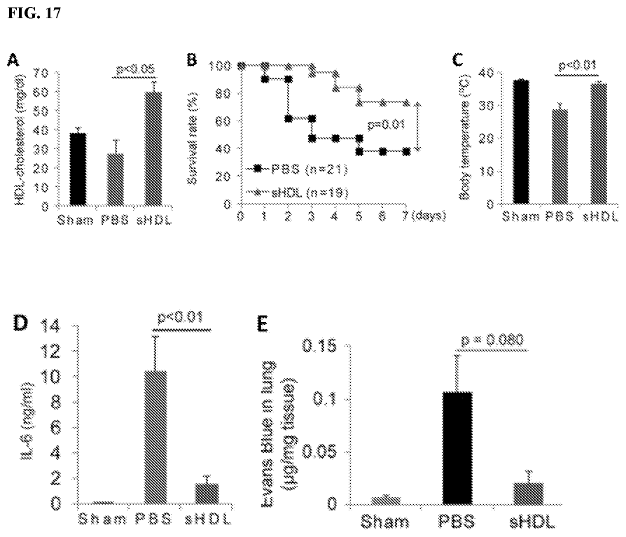

It has been shown that infusion of synthetic HDL (sHDL) nanoparticles in mice with infections increase overall survival, reduce pro-inflammatory cytokine release, inhibit endothelial activation and reduce organ damage. ETC-642 was administered to B6 mice 2h post cecal ligation and puncture (CLP), and showed that treatment significantly increased plasma HDL-cholesterol levels (FIG. 17A). Importantly, it was demonstrated that ETC-642 treatment significantly improved 7d survival rate in CLP mice (92%), compared to 57% in PBS-treated mice (FIG. 17B). It was also observed that septic mice treated with ETC-642 improved control of body temperature, a 9-fold decrease in plasma IL-6 levels and 5-fold lower Evans Blue leakage in the lung, compared to PBS treated mice (FIG. 17C-E).

sHDL Alters Lipid Raft Composition and could Reduce SARS-CoV-2 Cell Entry

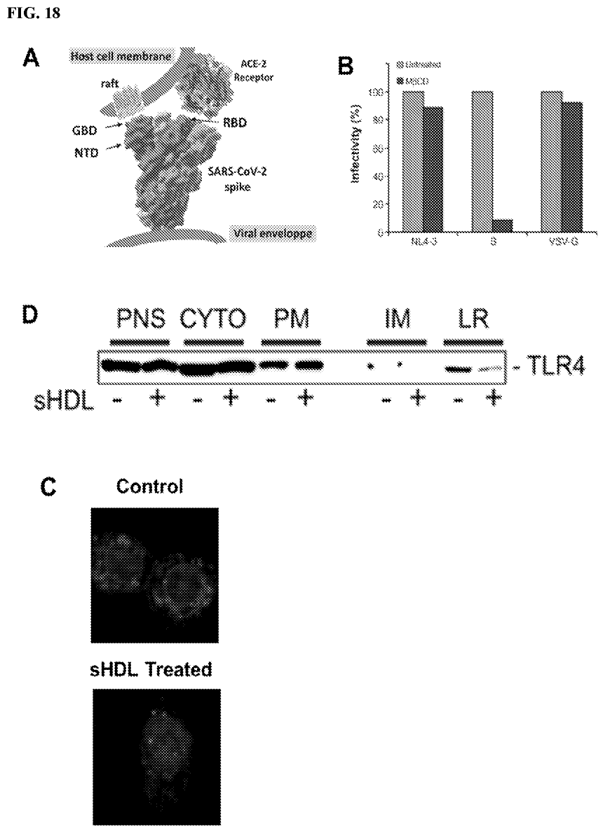

Lipid rafts are microdomains on cell membrane enriched with cholesterol and sphingolipids, as well as varieties of signaling proteins and virus receptors. Lipid rafts have been reported to be involved in cell entry of various virus including HIV and SARS-CoV. Recent molecular structure simulation studies showed that SARS-CoV-2 binds with both ACE-2 receptor and lipid rafts on cell membranes to initiate cell entry (FIG. 18). While it is still under debate whether ACE-2 receptors are directly associated with lipid rafts, multiple studies showed that lipid rafts depleter MβCD could significantly reduce or relocate ACE-2 receptors, resulting in inhibited SARS-CoV cell entry. However, the toxicity of MβCD limits its used in cellular studies. Similar to MβCD, our preliminary data showed that sHDL could significantly disrupt lipid rafts on cell surfaces, implying potential inhibitory effects of sHDL on cell entry of SARS-CoV-2.

### Synthetic HDL Offer Inhibition of Cytokine Release and Endothelial Protective Properties.

The overexuberant host inflammatory responses, which is manifested by excessive production of pro-inflammatory cytokines and chemokines, are one of the major factors causing tissue injury and organ failures in viral infections. It has been previously showed that SARS-CoV could activate NF-kB, leading to the production of pro-inflammatory cytokines. Inhibiting NF-kB activation was found to decrease pro-inflammatory cytokine levels, reduce lung pathological injuries and improve survival of SARS-CoV infected mice. Our preliminary data showed that sHDLs could significantly reduce NF-kB activation and secretion of pro-inflammatory cytokines on LPS-induced macrophages, suggesting potential immunoregulating functions of sHDLs in SARS-CoV-2 infection. Moreover, sHDLs reduced the overexpression of adhesion molecules and increased the production of eNOS on inflamed endothelia calls, suggesting beneficial regulatory effects on activated endothelial cells in virus infection.

### Endogenous HDL and Synthetic HDL Exhibit Anti-Thrombotic Properties.

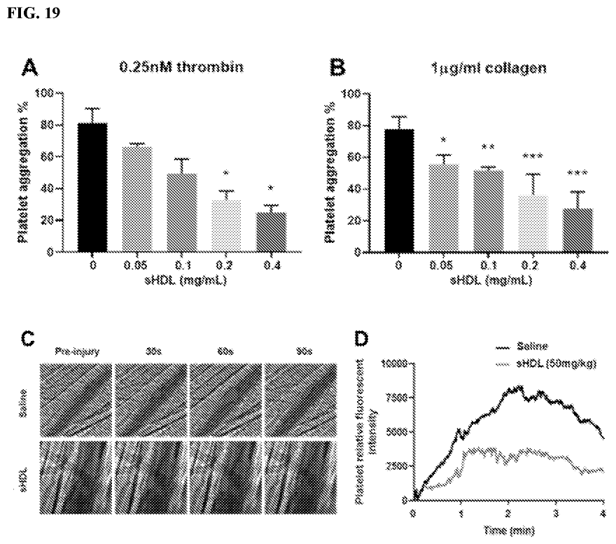

There is emerging clinical evidence suggesting an inverse correlation between HDL-C levels and the risk for atherothrombotic disorders. In 477 postmenopausal women with venous thrombosis the HDL-C levels were lower relative to age/sex matched controls. Patients with hyperlipoproteinemia show increased platelet reactivity and an enhanced thrombogenic potential. Anti-atherothrombotic properties of HDL has been generally attributed to the inhibition of platelet aggregation, and several sHDL infusions had been shown to reduce thrombus formation and arterial occlusion. The infusion of the plasma purified HDL, CSL-111 (80 mg/kg) to cardiovascular patients had shown a 50% reduction in the ex vivo platelet aggregation. The administration of a recombinant ApoA-1 Milano-based sHDL (ETC-216), shown inhibition of platelet aggregation and reduction of thrombus formation on an occlusive platelet-fibrin-rich thrombus rat model. Preliminary data show a dose-dependent inhibition of human platelet aggregation and a reduction of thrombus formation in the laser-induced thrombotic mouse model by a fully synthetic sHDL developed by us (FIG. 19). These clinical and preclinical funding suggest that sHDL might be a promising therapeutic strategy for inhibiting platelet hyperreactivity and reducing venous thrombosis and a diffuse DIC in COVID-19 patients.

### Novel Multifaceted Therapeutic Strategy for COVID-19

Provided herein is a novel strategy for COVID-19 by mimicking the protective functions of endogenous HDLs. The biomimetic sHDL not only could regulate functions of endothelium, platelets, and immune cells but may also directly interfere with virus infection process. The multifaceted therapeutic effects of sHDL make it a unique drug candidate.

Novel Therapeutical Application for sHDLs

The application of sHDL has been limited to cardiovascular diseases, where the formulation development is mainly focused on maximizing cholesterol efflux capacities. The present study proposes a new therapeutic application of sHDL for infectious diseases. The previously underinvestigated functions of sHDL, such as anti-infection, anti-inflammation, anti-thrombosis, and endothelial preservation functions, will be optimized by fine-tuning sHDL composition and dosing regime for COVID-19.

### Example III

sHDL can be Internalized Effectively by Platelets Both In Vitro and In Vivo.

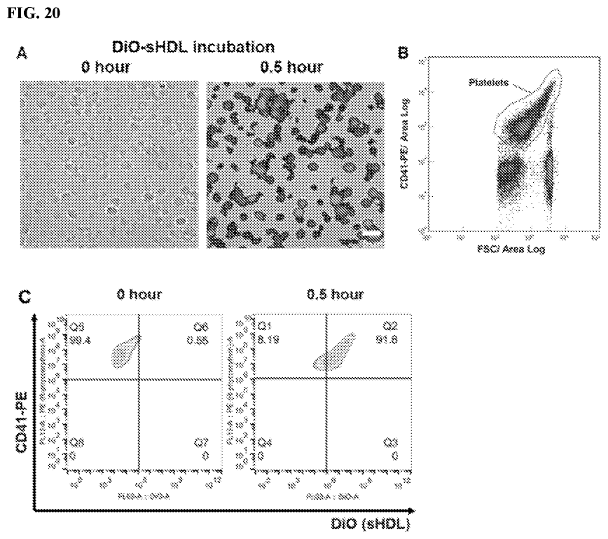

Isolated human platelets were incubated with DiO-sHDL (50 μg/mL 22A peptide, 2.5 μg/mL DiO) for 30 minutes, and the uptake of DiO-sHDL by human platelets was monitored by fluorescence microscopy. The results showed that sHDL was specifically internalized by human platelets (FIG. 20A). The specific uptake of sHDL by platelets was also verified using flow cytometry analysis of whole blood. C57BL/6J mice (10-12 weeks old, n=4/group) were dosed with DiO-sHDL (50 mg/kg of 22A peptide, 2.5 mg/kg of DiO) via tail vein injection, and blood was collected at predetermined time points. Platelets were stained with CD41-PE antibody (FIG. 20B). The fluorescent signal of sHDL in platelets was clearly observed after 30 minutes administration (FIG. 20C). These data indicate that platelets can effectively internalize sHDL, warranting the feasibility of sHDL to access its therapeutic target.

Differenes in Phospholipid Composition Impacts the Modulatory Effect of sHDL on Platelet Activation and Blood Coagulation In Vitro.

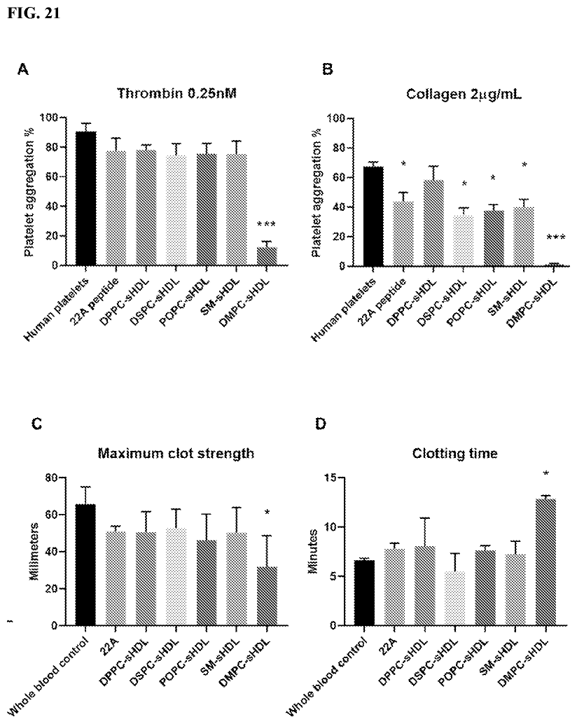

Washed human platelets were pretreated with various sHDLs consisting of an apolipoprotein mimetic peptide 22A and different phospholipids: 1,2-dipalmitoyl-sn-glycero-3-phosphocholine (DPPC), 1,2-distearoyl-sn-glycero-3-phosphocholine (DSPC), 1-palmitoyl-2-oleoyl-glycero-3-phosphocholine (POPC), sphingomyelin (SM), and 1,2-dimyristoyl-sn-glycero-3-phosphocholine (DMPC), respectively. After 30 minutes, the aggregation of DMPC-sHDL pretreated platelets was significantly blocked at 0.25 nM of thrombin relative to either non-treated platelets or other types of sHDL (FIG. 21A). Under the action of collagen, a weak platelet activator, DMPC-sHDL also showed the strongest inhibition of platelet aggregation. Other types of sHDL also presented differing degrees of inhibition on platelet aggregation (FIG. 21B). Furthermore, the effect of sHDL on blood coagulation in vitro was determined by a thromboelastographic analyzer. The results confirmed that only whole blood pretreated with DMPC-sHDL had an elongated clotting time and smaller clotting size (FIG. 21C-3D), compared to other types of sHDL. Such data shows that phospholipid composition of sHDL affects its modulatory effect on platelet activation, and DMPC-sHDL has the strongest inhibition of platelet aggregation.

sHDL Dose-Dependently Inhibits the Hyperactivation of Platelets and Attenuates Platelet Adhesion and Aggregation on Collagen Under Arterial Shear Forces in Whole Blood.

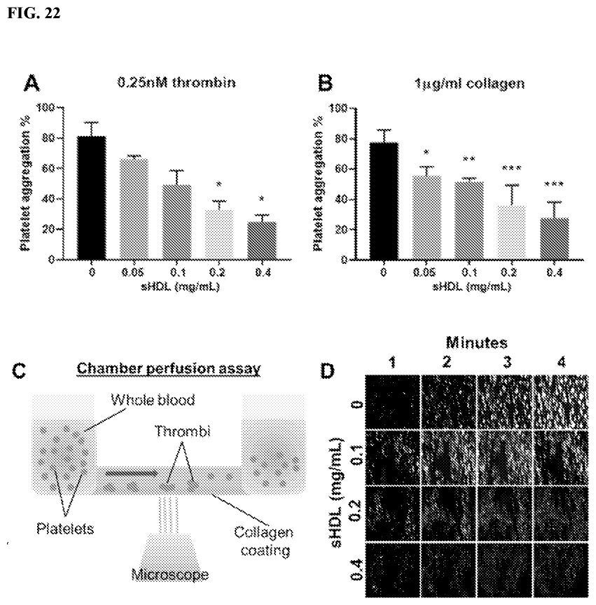

To determine range of sHDL's inhibition of platelet activation, we performed a platelet aggregation test and showed that sHDL inhibited platelet activation and aggregation in a dose-dependent manner (from 0.05 to 0.4 mg/mL) after stimulation of both thrombin and collagen (FIGS. 22A and 22B). To assess the effect of sHDL on human platelet adhesion and aggregation under flow, heparin-anticoagulated whole blood was incubated with sHDL (0.1, 0.2 and 0.4 mg/mL) for 30 minutes followed by perfusion at arterial shear (1800 s⁻¹) over a collagen-coated surface (schematically presented in FIG. 4C). As shown in FIG. 22D, platelets adhere, aggregate, and form a stable thrombus over the course of perfusion in non-treated whole blood. In contrast, whole blood treated with sHDL exhibited an attenuation of platelet adhesion, aggregation, and thrombus formation in a dose-dependent manner. Such data demonstrate that sHDL dose-dependently inhibits platelet activation and aggregation.

sHDL is Incorporated within Newly Formed Platelet-Rich Arterial Thrombi and Prevents Thrombosis Growth.

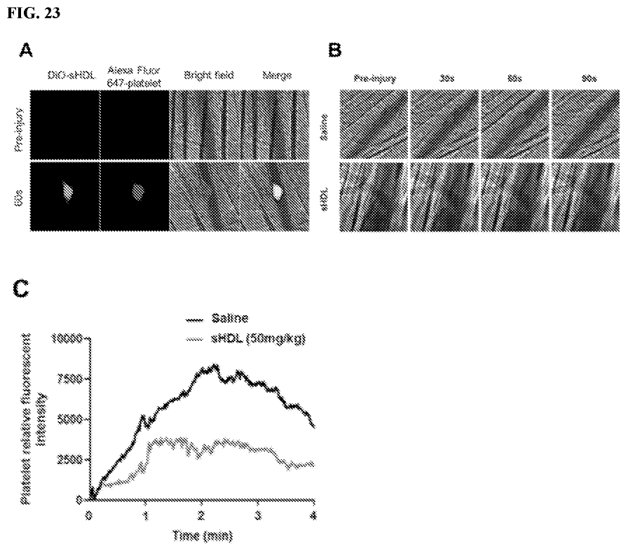

sHDL's excellent antiplatelet property led us to investigate whether intravenously injected sHDL could specifically be incorporated to platelet-rich thrombi formed in the artery, which is essential for sHDL to directly exert its antiplatelet effect and inhibit thrombus formation. A laser-induced arterial thrombus mouse model was established to test the targeting property of sHDL to thrombi. The results showed that sHDL well-localized in the newly formed platelet-rich thrombi (FIG. 23A). Moreover, intravenous administration of sHDL 24 hours prior to thrombus induction significantly impaired thrombus formation (FIGS. 23B and 23C). Collectively, such data strongly prove the potential of sHDL to inhibit platelet activation, aggregation and thrombosis formation.

### Example IV

This example demonstrates that HDL levels are reduced in septic patients.

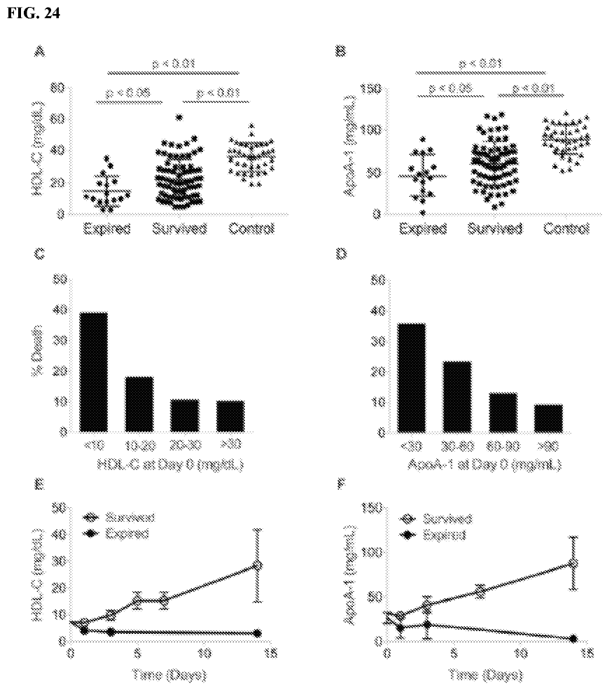

To better understand the role of HDL in sepsis, a clinical observation study was performed in one hundred twenty-four ICU patients, from whom 85 were with sepsis and 39 without sepsis, at the University of Michigan Hospital Intensive Care Unit. For analysis, patients were broken up into 3 groups: Non-sepsis controls (n=38), sepsis survivors (n=69), and non-surviving sepsis (n=16, also termed “sepsis expired”). HDL-cholesterol (HDL-C) levels at time of intake to the ICU (Day 0) were measured (FIG. 24A). Interestingly, HDL-C levels were markedly reduced in patients with sepsis versus non-sepsis controls, with sepsis-expired individuals displaying significantly lower HDL-C(14.99+/−9.576 mg/dL) than both sepsis-survivors (22.52+/−12.51 mg/dL, p<0.05) and non-sepsis controls (36.07+/−8.684 mg/dL, p<0.01). A similar trend was observed for ApoA1 levels, which also differed significantly between sepsis-expired (46.25+/−24.28 mg/mL), sepsis-survivors (60.87+/−25.42 mg/mL, p<0.05), and non-sepsis controls (88.84+/−17.64 mg/mL, p<0.01), as shown in FIG. 24B.

### Example V

This example demonstrates that HDL levels correlate with a poor prognosis.

Experiments were conducted to explore the relationship between HDL-C and survival among sepsis patients. Interestingly, it was found that patients entering the ICU with HDL-C levels <10 mg/dL had about a 2-fold increase in mortality compared to those with HDL-C >10 mg/dL (FIG. 24C), indicating that 1) decreased HDL may be a predictive factor for sepsis-related mortality; 2) HDL may play a protective role against sepsis; and 3) raising circulating HDL levels may provide a means to treat or prolong survival in septic patients. As expected, a similar trend held true for ApoA1 levels (FIG. 24D).

To further explore this notion, experiments were conducted looking at the 14-day HDL-C kinetics between sepsis survivors and expired patients with HDL-C<10 mg/dL on Day 0 (FIG. 24E). While many of the sepsis-expired patients did not make it beyond Day 3, a time-dependent increase in HDL-C in those surviving patients was observed. Again, a similar trend was seen for ApoA1 (FIG. 24F). Taken together, these data further support the notion that HDL-infusion therapy may increase a patient's chances of survival.

### Example VI

This example demonstrates that 22A/SM-DPPC preparation results in pure, homogenous peptide-lipid nanodiscs.

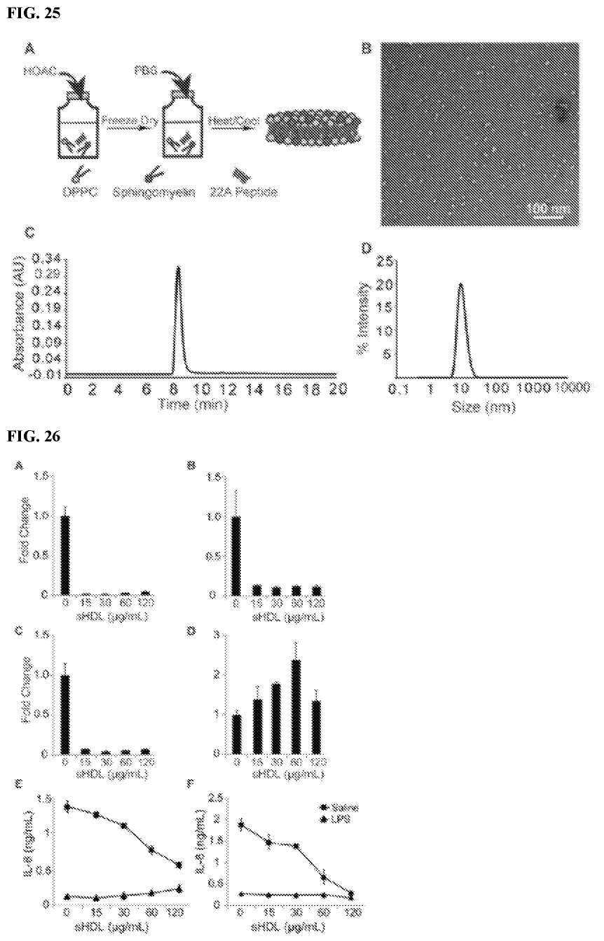

With clinical data in strong support of HDL as a protective entity against sepsis, experiments were conducted to test this notion in a laboratory-based setting. To do this, experiments used a synthetic HDL, 22A/SM-DPPC, and tested in Phase II clinical trials for the treatment of Acute Coronary Syndrome. 22A/SM-DPPC sHDL was made by a co-lyophilization technique (FIG. 25A) using the 22 amino acid Apolipoprotein A-I mimetic peptide (22A) and phospholipids 1,2-dipalmitoyl-sn-glycero-3-phosphocholine (DPPC) and sphingomyelin (SM). As seen in FIG. 25, sHDL was successfully produced with high homogeneity as seen by TEM (FIG. 25B), purity >99% as evidenced by GPC (FIG. 25C), with a size range of 10.43±3.283 nm with a PDI of 0.112 measured by DLS (FIG. 25D).

### Example VII

This example demonstrates that sHDL suppresses LPS-induced endothelial cell activation.

Experiments were conducted examining the beneficial effect (in any) of sHDL in endothelial cells because, in addition to macrophages, sepsis also manifests as a disorder of the endothelium. With the breakdown of endothelial barrier integrity comes infiltration of pro-inflammatory moieties to the tissues, initiating uncontrolled inflammation in otherwise healthy organs and eventually, in severe cases, organ failure. Several previous reports have shown sHDL to have restorative properties in damaged endothelium, and given the physiological relevance of the endothelium in sepsis, the next step was to assess sHDL protection against endothelial activation in vitro. Here, experiments used HUVECs activated with LPS (1 μg/mL) as our cell model, and examined the ability of sHDL to reduce cell adhesion molecule mRNA expression (VCAM-1, ICAM-1, and E-selectin), increase endothelial nitric oxide synthase (eNOS) mRNA levels, and decrease the production of pro-inflammatory cytokines IL-6 and IL-8. As expected, ICAM-1 (FIG. 26A), VCAM-1 (FIG. 26B), and E-selectin (FIG. 26C) mRNA expression levels were reduced >10-fold in HUVECs challenged with LPS in the presence of sHDL compared to PBS (p<0.01). Additionally, sHDL at concentrations of 30 and 60 μg/mL were also able in increase eNOS mRNA expression by 1.8 and 2.4-fold, respectively (FIG. 6D, p<0.05). There was no significant change in eNOS expression for cells treated with sHDL at 15 and 120 sg/mL compared to PBS-treated controls. Additionally, sHDL at all concentrations (15-120 μg/mL) was able to suppress production of IL-6 (FIG. 26E) and IL-8 (FIG. 26F) by >4-fold and >50-fold over PBS controls, respectively (p<0.01).

### Example VIII

This example presents the materials and methods for Examples IV-VII.

### Reagents

22A peptide (PVLDLFRELLNELLEALKQKLK (SEQ ID NO: 4)) was synthesized by Genscript (Piscataway, NJ) and purity was determined to be >95% by HPLC. Egg sphingomyelin (SM) and 1,2-dipalmitoyl-sn-glycero-3-phosphocholine (DPPC) were purchased from Avanti Polar Lipids (Alabaster, AL) and Nippon Oil and Fat (Osaka, Japan). LPS (E. coli 0111:B4) was purchased from Sigma Aldrich (St. Louis, MO). LPS (E. coli K12) was purchased from InvivoGen (San Diego, CA). Anti-SR-BI serum was custom made by Sigma-Genosys using a 15 amino acid-peptide derived from the C-terminal of human SR-BI. The authentication of the antibody has been verified by western blot using SR-BI null tissues. Anti-TLR4 was purchased from Santa Cruz (cat #sc-293072, CA). All other reagents were obtained from commercial suppliers and were of analytical grade or higher.

sHDL Preparation

Discoidal 22A/SM-DPPC sHDL nanoparticles were made by co-lyophilization followed by thermal cycling. Briefly, 22A peptide and phospholipids were combined and dissolved in glacial acetic acid at a 22A:SM:DPPC ratio of 1:1:1 by weight. The resulting solution underwent rapid freezing in liquid nitrogen and immediately placed on a shelf freeze-dryer (Labconco) overnight to remove the acid. Once dried, the lyophilized powder was reconstituted in warm 1× Phosphate Buffered Saline (PBS) to the desired final peptide concentration and vortexed to completely dissolve, forming a cloudy white solution. The resulting solution was subjected to 3 heat/cool cycle, each cycle consisting of 10 minutes heating at 55° C. and 10 minutes cooling at room temperature (above and below the transition temperature of the lipids, respectively). By the end of 3 cycles the solution had turned from cloudy to clear, indicating formation of sHDL nanoparticles. pH of sHDL solution was adjusted to 7.4 using NaOH and 0.2 μm sterile filtered.

sHDL Characterization

Quality of 22A/SM-DPPC sHDL particles was assessed using the following analytical techniques. Size distribution was determined by dynamic light scattering (DLS) on a Malvern Nano ZSP (UK), and purity of particles was determined by gel permeation chromatography (GPC) with UV detection at 220 using a Tosoh TSK gel G3000SWx1 column (Tosoh Bioscience, King of Prussia, PA) on a Waters HPLC.

### Cell Culture

Primary Human Umbilical Vein Endothelial Cells (HUVEC) from pooled donors were grown in 0.2% gelatin Type B (Sigma) coated tissue culture vessels containing Clonetics™ EGM-2 Complete Media (Lonza). Cells between passages 3-5 were used in all experiments. RAW 264.7 murine macrophages (ATCC* TIB-71™) were cultured in Dulbecco's Modified Eagle Medium (DMEM) containing 10% fetal bovine serum. HEK-Blue cells, which stably express CD14, MD2, NF-κB reporter and TLR4 or TLR2, were from InvivoGen. All incubations were performed in a 37° C. incubator under 5% CO₂ atmospheric conditions.

### Endothelial LPS/TNF-α-Induced Cytokine Expression Analysis

HUVECs were seeded into well-plates and grown to 90% confluency. Cells were washed twice with 1×PBS and incubated with LPS (0111:B4, 100 μg/mL) or TNF-α, (1 ng/mL) in the presence of 22A/SM-DPPC (15, 30, 60, or 120 μg/mL peptide), or matching concentrations of 22A peptide, SM-DPPC liposomes, or PBS 16 hours. The concentrations of cytokines IL-6 and IL-8 in the supernatants were quantified using ELISA.

### Gene Expression Analysis

HUVECs were seeded into well-plates and grown to 90% confluency. Cells were treated with LPS (100 μg/mL) in the presence of either 22A/SM-DPPC (15, 30, 60, or 120 μg/mL peptide), or matching concentrations of 22A peptide, SM-DPPC liposomes, or PBS for 16 hours. After incubation, cells were washed twice in PBS and cells lysed in radioimmunoprecipitation buffer (50 mM Tris, 150 mM NaCl, 1% SDS, 0.5% sodium deoxycholate, 1% Triton X-100) containing cOmplete™ EDTA-free protease inhibitor cocktail (Roche). Total RNA was extracted using RNeasy mini kit (Qiagen) and reverse transcribed to cDNA using iScript cDNA synthesis kit (BioRad). Gene expression was determined by RT-qPCR on a StepOnePlus™ Real-Time PCR System (Applied Biosystems) using TaqMan assays for ICAM-1, VCAM-1, eNOS, and E-Selectin (Thermo Fisher). Data was normalized to endogenous GAPDH expression and fold change in gene expression was calculated using the ΔΔC_{T} method.

### Patients

Patients were recruited at the University of Michigan Medical ICU at the onset of sepsis, between September 2001 and April 2004. Sepsis was defined as described by the American-European Sepsis Consensus Conference (see inclusion/exclusion criteria below). Prior to entry into the study, an informed consent was properly obtained from the patient or the patient's legally acceptable representative. The study was approved by the institutional review board of the University of Michigan Medical School, Ann Arbor, MI. At the time of entry, a complete medical/sepsis history and physical examination were obtained from each subject. The following data were recorded: APACHE III (Acute Physiology, Age, Chronic Health Evaluation) score, results of chest X-ray, electrocardiogram, ventilator parameters, positive culture, antigenic or nucleic acid assay results from any suspected source of sepsis, urinary output, administration of neuromuscular blocking agents, antibiotics, vasopressors and sedatives during the preceding 24 hours. The APACHE III score was assigned once a day by a trained nurse at the ICU unit based on the previous 24 hours of evaluation. From the laboratory studies recorded arterial blood gases, most recent pulmonary artery systolic, diastolic, and wedge pressure (where available), blood profile, serum electrolytes, glucose, bilirubin, and albumin were recorded.

### Patient Inclusion/Exclusion Criteria

For the sepsis patients, experiments were conducted which enrolled subjects of both sex and aged ≥18 years that had at least two signs of systemic inflammatory response syndrome (SIRS). SIRS was defined by core, rectal, axillary, or oral temperature ≥38° C. or otherwise unexplained core or rectal temperature of ≤36° C.; heart rate ≥90 beats per minute; respiratory rate ≥20 per minute or PaC02≤32 mmHg or the subject was on a ventilator; WBC≥12,000/mm³ or ≤4,000/mm³ or ≥10% immature neutrophils (bands). The source of sepsis was documented by culture, Gram stain or nucleic acid assay of blood, or normally sterile body fluid positive for a pathogenic microorganism that constituted the reason for systemic therapy with anti-infectives; chest radiography consistent with a diagnosis of pneumonia that constituted the reason for systemic therapy with anti-infectives; clearly verifiable focus of infection identified, e.g. perforated bowel with the presence of free air or bowel contents in the abdomen found at surgery; wound with purulent drainage. Experiments were conducted which also enrolled patients with septic shock (hypotension despite adequate fluid resuscitation (systolic BP≤90 mmHg, mean arterial BP≤65 mmHg) and need for vasopressors) or with organ dysfunction/hypoperfusion as a result of sepsis (e.g. pulmonary dysfunction—PaO₂/FIO₂<250, or <200 in the presence of pneumonia or other localizing lung disease; metabolic acidosis—pH≤7.30 or increased plasma lactate levels; oliguria—urine output<0.5 ml/kg/hr for a minimum of two consecutive hours in the presence of adequate fluid resuscitation; thrombocytopenia—platelet count of <100,000 cells/mm³ without other causes of thrombocytopenia; acute alteration in mental status). Subjects were excluded for the following criteria: pregnancy confirmed by urine or serum test; significant liver disease as defined by fulfillment of Child-Pugh Grade C or known esophageal varices; HIV infection with CD4+ count <200; Prednisone therapy >20 mg/day (or equivalent), cytotoxic therapy within 3 weeks prior to screening; confirmed, clinically-evident acute pancreatitis; extracorporeal support of gas exchange at the time of study entry; or receipt of an investigational drug within 30 days prior to study enrollment. For the non-septic control group experiments were conducted which enrolled subjects of either sex and age >18 years admitted to the ICU for disorders other than sepsis, who did not have any of the exclusion criteria outlined above.

### Patient Characteristics

A total of 124 patients from the CCMU at the University of Michigan Medical Center were recruited in this study, from whom 85 were patients with sepsis and 39 without sepsis.

### Blood Sampling and Analysis

Blood samples (15-20 ml) were taken in heparinized tubes within 24 hours from the onset of sepsis, and 1, 3, 7 and 14 days post entry. Plasma was separated by centrifugation at 4° C., aliquoted and stored at −80° C. before analysis. HDL cholesterol (Roche kit 3030067), total cholesterol (Roche kit 450061), triglycerides (Roche kit 1488899), apoA-I (Wako, Richmond, VA kit 991-27201), aspartate aminotransferase (AST; Roche kit 450064), were analyzed on a Hitachi 912 clinical chemistry autoanalyzer (Roche Diagnostics Corporation, Indianapolis, IN) by the Clinical Pathology Laboratory, Department of Drug Safety Evaluation at Esperion Therapeutics, a Division of Pfizer Global Research and Development, Ann Arbor, MI.

### Example IX

### Example IX

This example describes the preparation and characterization of 22A-phospholipids complexes synthetic HDL (sHDL).

Result

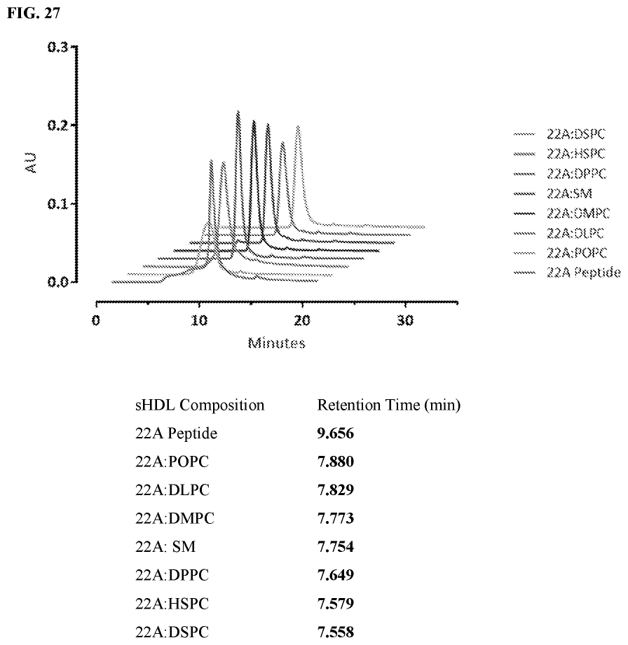

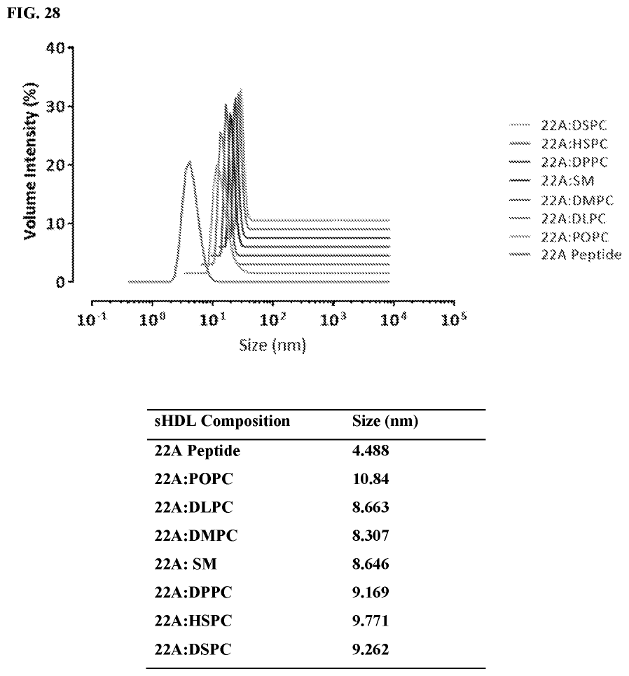

sHDLs were synthesized via co-lyophilization. Peptide and phospholipids were synthesized at 1:2 w/w ratio. 22A peptide displayed a retention time of 9.66 min, whereas sHDL complexes eluted at approximately 7 min (FIG. 27). The purity of all sHDL complexes exhibited >98%. Also, 22A peptide displayed a diameter of 4.49 nm whereas sHDL complexes exhibited diameters ranging from 8-10 nm (FIG. 28). Diameters of sHDL complexes were similar to that of native HDL, which also ranges from 8-10 nm. These data show that sHDL complexes have size distributions equivalent to that of native HDL. Phase transition temperature of sHDL complexes displayed similar trend compared to transition temperature of lipid, however average of 3.7° C. increased when lipids were incorporated with 22A peptide in 1:2 ratio (Table 6). Overall characterizations are simply described in Table 7.

TABLE 6
Transition Temperature (Tm) of sHDL complexes

| Name | Tm of Lipid* (° C.) | Tm of sHDL (° C.) | Difference (° C.) |
|---|---|---|---|
| 22A:POPC | −3 | 0.5 ± 0.5 | +3.5 ± 0.5 |
| 22A:DLPC | 0 | 0.3 ± 0.28 | +0.3 ± 0.28 |
| 22A:DMPC | 23 | 27 ± 0 | +4.0 ± 0 |
| 22A:SM | Ca. 40** | 44.07 ± 0.11 | +6.07 ± 0.11 |
| 22A:DPPC | 41 | 45.37 ± 0.37 | +4.37 ± 0.37 |
| 22A:HSPC | 52** | 56.86 ± 0.57 | +4.86 ± 0.57 |
| 22A:DSPC | 55 | 57.76 ± 1.25 | +2.76 ± 1.25 |

*Transition temperature of lipids were obtained from product description of manufacturer.
**Bavelloni A, Piazzi M, Raffini M, Faenza I, Blalock WL. Prohibitin 2: At a communications crossroads. IUBMB Life. 2015; 67(4): 239-54.

TABLE 7
Overall characterization of sHDL complexes

| sHDL | Tm | Size | Retention | Purity | Lipid Alkyl |
|---|---|---|---|---|---|
| Components | (° C.) | (nm) | Time (min) | (%) | Composition |
| 22A PEPTIDE | — | 4.488 | 9.656 | 77.73 | — |
| 22A:POPC | 0.5 | 10.84 | 7.880 | 99.30 | 16:0/18:1 |
| 22A:DLPC | 0.3 | 8.663 | 7.829 | 98.90 | 12:0/12:0 |
| 22A:DMPC | 27 | 8.307 | 7.773 | 99.29 | 14:0/14:0 |
| 22A:SM | 44.07 | 8.646 | 7.754 | 99.19 | 16:0/18:0, 22:0 |
| 22A:DPPC | 45.37 | 9.169 | 7.649 | 98.48 | 16:0/16:0 |
| 22A:HSPC | 56.86 | 9.771 | 7.579 | 99.12 | 18:0/16:0 |
| 22A:DSPC | 57.76 | 9.262 | 7.558 | 99.20 | 18:0/18:0 |

### Example X

This example describes that inhibition of LPS-induced NF-κB activation is dependent to lipid component of sHDL complexes.

Results

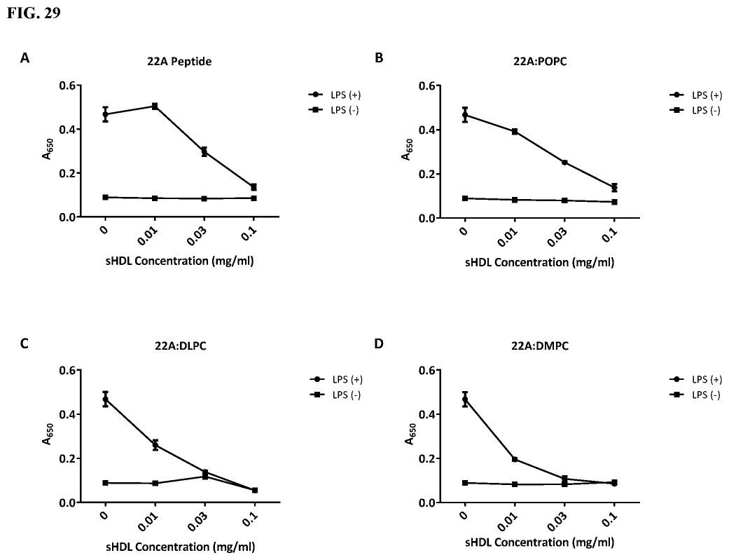

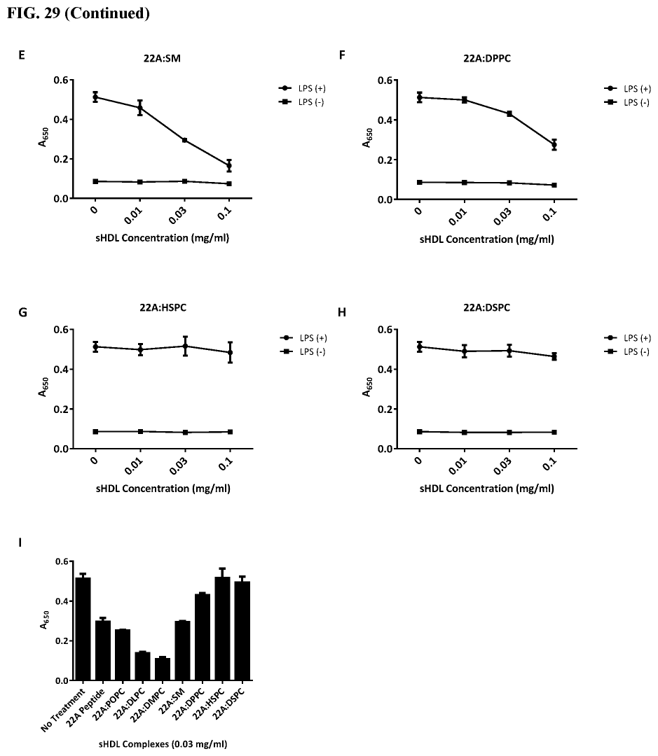

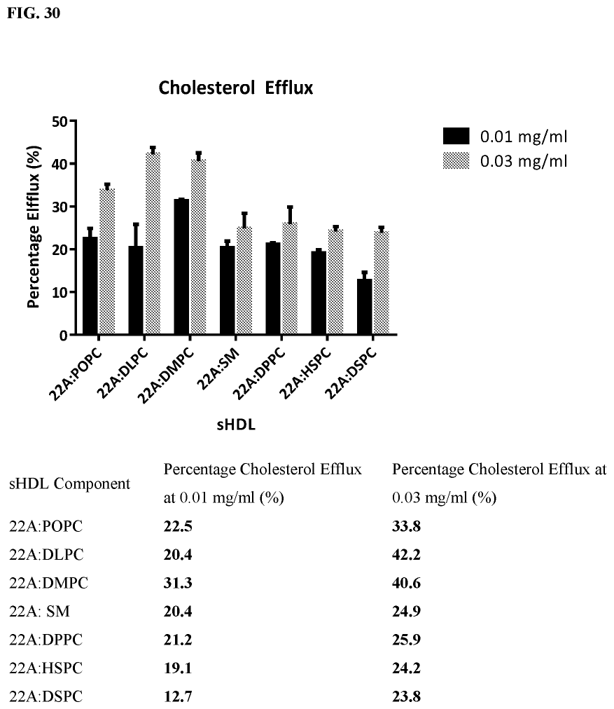

HEK-Blue cells were used to determine whether sHDL complex neturalizes LPS and inhibits interaction between LPS and TLR4. Once LPS binds to TLR4, TLR4 becomes activated and stimulates NF-κB activation resulting in a high absorbance value at 650 nm. Experiments were performed using sHDL concentrations at 0.01, 0.03, and 0.1 mg/ml and levels of NF-κB activation was measured (FIG. 29). 22A:DLPC (C) and 22A:DMPC (D) complexes showed significant inhibition of NF-kB activation at both 0.01 and 0.03 mg/ml (I). At 0.1 mg/ml concentration, 22A peptide (A), 22A:POPC (B), 22A:DLPC (C), 22A:DMPC (D), 22A:SM (E), and 22A:DPPC (F) suppressed the activation of NF-kB. sHDL complexes that showed inhibition of NF-kB activation at either concentration of 0.03 or 0.1 mg/ml exhibited transition tmerature of lower or close to the temperature of incubator which was 37° C.

### Example XI

This example describes that cholesterol efflux is dependent to lipid component of sHDL complexes.

Results

HDL have intrinsic property of uptaking excessive cholesterol from macrophages. To determine whether lipid component of sHDL complexes affect efflux of cholesterol, we labeled RAW 264.7 macrophages with [³H] cholesterol. The cells were then treated with sHDL complexes for 18 hours and radioactive counts in media and cell fractions were measured by liquid scintillation counting. 22A:DLPC and 22A:DMPC displayed more than 40% of cholesterol efflux at 0.03 mg/ml while other sHDL complexes displayed less than 30% of cholesterol efflux (FIG. 30). Also, more cholesterol was effluxed at higher concentration of sHDL complexes. It is worth noting that cholesterol efflux was higher in sHDL complexes that exhibited transition temperature lower to the temperature of incubator which was 37° C.

### Example XII

This example presents the materials and methods for Examples IX-XI.

Materials

22A (PVLDLFRELLNELLEALKQKLK (SEQ ID NO: 4)) was synthesized by GenScript (Piscataway, NJ) and purity was ˜85% as determined by HPLC. 1-palmitoyl-2-oleoyl-sn-glycero-3-phosphocholine (POPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), hydrogenated soybean phosphatidylcholine (HSPC), 1,2-distearoyl-sn-glycero-3-phosphocholine (DSPC) were purchased from Nippon Oil and Fat (Osaka, Japan). Egg-sphingomyelin (SM) was purchased form Avanti Polar Lipids (Alabaster, AL). Lipopolysaccharide (LPS) (Escherichia coli serotype K-12) was from InvivoGen (San Diego, CA). The HEK-Blue™ TLR4 cells and HEK-Blue™ Detection were purchased from InvivoGen (San Diego, CA).

Cell Cultures

All cell lines were cultured at 37° C. in a humidified 5% CO2 incubator. RAW 264.7 murine macrophages (ATCC® TIB-71™) were cultured in Dulbecco's Modified Eagle Medium (DMEM) containing 10% fetal bovine serum (FBS). HEK-Blue™ cells, which stably express CD14, MD2, NF-κB reporter and human TLR4, were purchased from InvivoGen and grown in DMEM containing 10% FBS. HEK-Blue™ cells express the secreted embryonic alkaline phosphatase (SEAP) reporter gene under the control of the NF-κB promotor, which enables the quantification of cell activation by measuring SEAP activity in medium containing specific enzyme substrates.

Preparation of 22A-Phospholipids Complexes Synthetic HDL (sHDL)

sHDLs were synthesized via co-lyophilization. Briefly, peptide and phospholipids were dissolved in glacial acetic acid, mixed at 1:2 w/w ratio, flash frozen and lyophilized for several days. The resulting powder was then hydrated with phosphate buffered saline (PBS) and thermal-cycled 3-5 times above and below the transition temperature of lipids for 10 minutes each to facilitate peptide-lipid binding (Table 8). pH of sHDL solutions were adjusted to 7.4 using NaOH and 0.2 μm sterile filtered.

TABLE 8
Thermo-cycling temperature for sHDL synthesis

| | Lipid | | |
|---|---|---|---|
| | Transition | Cooling | Heating |
| sHDL | Temperature* | Temperature | Temperature |
| Component | (° C.) | (° C.) | (° C.) |
| 22A:POPC | −3 | 0 | Room Temperature |
| 22A:DLPC | 0 | 0 | Room Temperature |
| 22A:DMPC | 23 | Room Temperature | 50 |
| 22A:SM | 40** | Room Temperature | 50 |
| 22A:DPPC | 41 | Room Temperature | 50 |
| 22A:HSPC | 52** | Room Temperature | 60 |
| 22A:DSPC | 55 | Room Temperature | 60 |

*Transition temperature of lipids were obtained from product description of manufacturer.
**Bavelloni A, Piazzi M, Raffini M, Faenza I, Blalock WL. Prohibitin 2: At a communications crossroads. IUBMB Life. 2015; 67(4): 239-54.

Characterization of 22A-Phospholipids Complexes sHDL

The quality of resulting sHDL complexes were analyzed by following analytical techniques. The purity of sHDL complexes were determined by gel permeation chromatography (GPC), with UV detection at 220 nm, using a Tosoh TSK gel G3000SWxl column (Tosoh Bioscience, King of Prussia, PA). The sHDL diameters were determined by dynamic light scattering (DLS), using a Zetasizer Nano ZSP (Malvern Instruments, Westborough, MA) and the volume intensity average values were reported. Transition temperature of sHDL complexes were analyzed by differential scanning calorimetry (DSC) using Nano DSC (TA Instruments, New Castle, DE).

LPS-Induced NF-κB Expression in HEK-Blue Cells

The HEK-Blue cell system (InvivoGen) was used to analyze neutralization of the LPS-induced inflammatory response. HEK-Blue cells stably express reporter-linked human TLR4, CD14, MD2, and a NF-κB and are designed for studying the stimulation of human TLR4. Briefly, HEK-Blue cells were cultured in Dulbecco's Modified Eagle's Medium (DMEM) containing 10% low endotoxin fetal bovine serum (FBS) and selective antibiotics according to the manufacturer's instructions (InvivoGen). Growth medium was discarded and cells were resuspended in HEK-Blue Detection medium. Cells were seeded at 25,000 cells per well. The cells were treated with sHDL at 0.01, 0.03, or 0.1 mg/ml and 2 ng/ml of LPS. The cells were then incubated for 18 hours. LPS binding to TLR4 results in activation of NF-κB reporter expression, causing the HEK-Blue detection medium to turn blue. The blue color was quantified by measuring absorption at 650 nm using a SpectraMax M3 plate reader (Molecular Devices, Sunnyvale, CA).

Cholesterol Efflux

RAW 264.7 macrophages were grown in DMEM containing 10% low endotoxin FBS. Then, 1×10⁵ cells were plated in 24 well plates and grown for 24 hours. Cells were washed with PBS pH 7.4 once and labeled for 24 hours in growth medium containing 1 ρCi of [³H] cholesterol/mL. The cells were then washed with PBS and sHDL was added at concentrations of 0.01 or 0.03 mg/ml peptide in DMEM-BSA media. After 18 h of incubation, media were collected and cells lysed in 0.4 ml of 0.1% SDS and 0.1 N NaOH. Radioactive counts in media and cell fractions were measured by liquid scintillation counting, and percent cholesterol effluxed was calculated by dividing the media count by the sum of the media and cell counts.

### Example XIII

This example describes methods of sHDL production.

Solubilization Method

Peptide and lipids are weighed out separately and dissolved in warm aqueous buffer (i.e. phosphate, carbonate-bicarbonate, saline, water), with vortexing to achieve a homogeneous suspension. Components are then added together at the desired final weight ratio (1:1-1:4) and briefly vortexed. The resulting suspension is thermal-cycled 3-5 times above and below the transition temperature of the lipids, holding for 10 minutes at each temperature, in order to form a clear solution. The solution is then adjusted to pH 7.4 and filtered through a 0.2 μm porous membrane.

Thin Film Method

Lipids are weighed out and completely dissolved in chloroform. Chloroform is then evaporated under a gentle stream of nitrogen (or other inert gas), while gently rotating the vial in order to create a thin lipid film on the wall of the vial. Residual solvent is evaporated by placing the vial in a vacuum oven at ambient temperature overnight. A solution of peptide is made by dissolving the desired amount of peptide in aqueous buffer (i.e. phosphate, carbonate-bicarbonate, saline, water), followed by warming such that the temperature of the peptide solution is above the transition temperature of the lipids used for sHDL production. The warm peptide solution is then added to the lipid film followed by vortexing to completely hydrate the lipid film. Three to five cycles of heating and cooling above and below the transition temperature of the lipids are performed, holding each temperature for 10 minutes. The resulting sHDL solution is pH adjusted to 7.4 and filtered through a 0.2 μm porous membrane.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes. In particular, the following references numerically denoted within the application are as follows:
1. Singer, M., C. S. Deutschman, C. W. Seymour, M. Shankar-Hari, D. Annane, M. Bauer, R. Bellomo, G. R. Bernard, J.-D. Chiche, C. M. Coopersmith, R. S. Hotchkiss, M. M. Levy, J. C. Marshall, G. S. Martin, S. M. Opal, G. D. Rubenfeld, T. van der Poll, J.-L. Vincent, D. C. Angus, T. Van Der Poll, J.-L. Vincent, and D. C. Angus. 2016. The Third International Consensus Definitions for Sepsis and Septic Shock (Sepsis-3). JAMA. 315: 801-810.
2. Medzhitov, R, and C. Janeway. 2000. Innate immunity. N. Engl. J. Med. 343: 338-44.
3. Poltorak, A., X. He, I. Smimova, M. Y. Liu, C. Van Huffel, X. Du, D. Birdwell, E. Alejos, M. Silva, C. Galanos, M. Freudenberg, P. Ricciardi-Castagnoli, B. Layton, and B. Beutler. 1998. Defective LPS signaling in C3H/HeJ and C57BL/10ScCr mice: mutations in Tlr4 gene. Science. 282: 2085-8.
4. Maeshima, N., and R. C. Fernandez. 2013. Recognition of lipid A variants by the TLR4-MD-2 receptor complex. Front. Cell. Infect. Microbiol. 3: 3.
5. Lu, Y.-C., W.-C. Yeh, and P. S. Ohashi. 2008. LPS/TLR4 signal transduction pathway. Cytokine. 42: 145-51.
6. Pirillo, A., A. L. Catapano, and G. D. Norata. 2015. In Handbook of experimental pharmacology. pp. 483-508., Springer, Cham.
7. Guo, L., Z. Zheng, J. Ai, B. Huang, and X.-A. Li. 2014. Hepatic scavenger receptor BI protects against polymicrobial-induced sepsis through promoting LPS clearance in mice. J. Biol. Chem. 289: 14666-73.
8. Zhu, X., J. S. Owen, M. D. Wilson, H. Li, G. L. Griffiths, M. J. Thomas, E. M. Hiltbold, M. B. Fessler, and J. S. Parks. 2010. Macrophage ABCA1 reduces MyD88-dependent Toll-like receptor trafficking to lipid rafts by reduction of lipid raft cholesterol. J. Lipid Res. 51: 3196-206.
9. De Nardo, D., L. I. Labzin, H. Kono, R. Seki, S. V Schmidt, M. Beyer, D. Xu, S. Zimmer, C. Lahrmann, F. a Schildberg, J. Vogelhuber, M. Kraut, T. Ulas, A. Kerksiek, W. Krebs, N. Bode, A. Grebe, M. L. Fitzgerald, N. J. Hemandez, B. R. G. Williams, P. Knolle, M. Kneilling, M. Röcken, D. Lütjohann, S. D. Wright, J. L. Schultze, and E. Latz. 2014. High-density lipoprotein mediates anti-inflammatory reprogramming of macrophages via the transcriptional regulator ATF3. Nat. Immunol. 15: 152-60.
10. Wang, Y., Y. Wang, S. Jia, Q. Dong, Y. Chen, S. Lu, and L. Hou. 2017. Effect of lipid-bound apolipoprotein A-I cysteine mutant on ATF3 in RAW264.7 cells. Biosci. Rep. 37: BSR20160398.
11. Smith, C. K., N. L. Seto, A. Vivekanandan-Giri, W. Yuan, M. P. Playford, Z. Manna, S. A. Hasni, R. Kuai, N. N. Mehta, A. Schwendeman, S. Pennathur, and M. J. Kaplan. 2017. Lupus high-density lipoprotein induces proinflammatory responses in macrophages by binding lectin-like oxidised low-density lipoprotein receptor 1 and failing to promote activating transcription factor 3 activity. Ann. Rheum. Dis. 76: 602-611.
12. Alvarez, C., and A. Ramos. 1986. Lipids, lipoproteins, and apoproteins in serum during infection. Clin. Chem. 32: 142-5.
13. Sammalkorpi, K., V. Valtonen, Y. Kerttula, E. Nikkilä, and M. R. Taskinen. 1988. Changes in serum lipoprotein pattern induced by acute infections. Metabolism. 37: 859-65.
14. Khosla, S. N., N. Goyle, and R. K. Seth. 1991. Lipid profile in enteric fever. wJ. Assoc. Physicians India. 39: 260-2.
15. van Leeuwen, H. J., E. C. J. M. Heezius, G. M. Dallinga, J. A. G. van Strijp, J. Verhoef, and K. P. M. van Kessel. 2003. Lipoprotein metabolism in patients with severe sepsis. Crit. Care Med. 31: 1359-66.
16. Feingold, K. R., I. Hardardottir, R. Memon, E. J. Krul, A. H. Moser, J. M. Taylor, and C. Grunfeld. 1993. Effect of endotoxin on cholesterol biosynthesis and distribution in serum lipoproteins in Syrian hamsters. J. Lipid Res. 34: 2147-58.
17. Gordon, B. R., T. S. Parker, D. M. Levine, S. D. Saal, J. C. Wang, B. J. Sloan, P. S. Barie, and A. L. Rubin. 1996. Low lipid concentrations in critical illness: implications for preventing and treating endotoxemia. Crit. Care Med. 24: 584-9.
18. Chien, J.-Y., J.-S. Jerng, C.-J. Yu, and P.-C. Yang. 2005. Low serum level of high-density lipoprotein cholesterol is a poor prognostic factor for severe sepsis. Crit. Care Med. 33: 1688-93.
19. Tsai, M.-H., Y.-S. Peng, Y.-C. Chen, J.-M. Lien, Y.-C. Tian, J.-T. Fang, H.-H. Weng, P.-C. Chen, C.-W. Yang, and C.-S. Wu. 2009. Low serum concentration of apolipoprotein A-I is an indicator of poor prognosis in cirrhotic patients with severe sepsis. J. Hepatol. 50: 906-15.
20. Morin, E. E., L. Guo, A. Schwendeman, and X. A. Li. 2015. HDL in sepsis—risk factor and therapeutic approach. Front. Pharmacol. 6: 1-9.
21. Gupta, H., L. Dai, G. Datta, D. W. Garber, H. Grenett, Y. Li, V. Mishra, M. N. Palgunachari, S. Handattu, S. H. Gianturco, W. A. Bradley, G. M. Anantharamaiah, and C. R. White. 2005. Inhibition of lipopolysaccharide-induced inflammatory responses by an apolipoprotein AI mimetic peptide. Circ. Res. 97: 236-243.
22. Dai, L., G. Datta, Z. Zhang, H. Gupta, R. Patel, J. Honavar, S. Modi, J. M. Wyss, M. Palgunachari, G. M. Anantharamaiah, and C. R. White. 2010. The apolipoprotein A-I mimetic peptide 4F prevents defects in vascular function in endotoxemic rats. J. Lipid Res. 51: 2695-705.
23. Zhang, Z., G. Datta, Y. Zhang, A. P. Miller, P. Mochon, Y.-F. Chen, J. Chatham, G. M. Anantharamaiah, and C. R. White. 2009. Apolipoprotein A-I mimetic peptide treatment inhibits inflammatory responses and improves survival in septic rats. Am. J. Physiol. Heart Circ. Physiol. 297: H866-H873.
24. Moreira, R. S., M. Irigoyen, T. R. Sanches, R. A. Volpini, N. O. S. Camara, D. M. Malheiros, M. H. M. Shimizu, A. C. Seguro, and L. Andrade. 2014. Apolipoprotein A-I mimetic peptide 4F attenuates kidney injury, heart injury, and endothelial dysfunction in sepsis. Am. J. Physiol. Regul. Integr. Comp. Physiol. 307: R514-24.
25. Imai, T., T. Fujita, and Y. Yamazaki. 2003. Beneficial effects of apolipoprotein A-I on endotoxemia. Surg. Today. 33: 684-687.
26. Yan, Y. jie, Y. Li, B. Lou, and M. ping Wu. 2006. Beneficial effects of ApoA-I on LPS-induced acute lung injury and endotoxemia in mice. Life Sci. 79: 210-215.
27. Zhang, X., L. Wang, and B. Chen. 2015. Recombinant HDL (Milano) protects endotoxin-challenged rats from multiple organ injury and dysfunction. Biol. Chem. 396: 53-60.
28. Darabi, M., I. Guillas-Baudouin, W. Le Goff, M. J. Chapman, and A. Kontush. 2016. Therapeutic applications of reconstituted HDL: When structure meets function. Pharmacol. Ther. 157: 28-42.
29. Ulevitch, R. J., and A. R. Johnston. 1978. The modification of biophysical and endotoxic properties of bacterial lipopolysaccharides by serum. J. Clin. Invest. 62: 1313-24.
30. Wurfel, M. M., E. Hailman, and S. D. Wright. 1995. Soluble CD14 acts as a shuttle in the neutralization of lipopolysaccharide (LPS) by LPS-binding protein and reconstituted high density lipoprotein. J. Exp. Med. 181: 1743-54.
31. Levine, D. M., T. S. Parker, T. M. Donnelly, A. Walsh, and a L. Rubin. 1993. In vivo protection against endotoxin by plasma high density lipoprotein. Proc. Natl. Acad. Sci. U.S.A 90: 12040-12044.
32. Davidson, W. S., K. L. Gillotte, S. Lund-Katz, W. J. Johnson, G. H. Rothblat, and M. C. Phillips. 1995. The effect of high density lipoprotein phospholipid acyl chain composition on the efflux of cellular free cholesterol. J. Biol. Chem. 270: 5882-90.
33. Khan, M., S. Drake, J. Crockatt, and J. Dasseux. 2003. Single-dose intravenous infusion of ETC-642, a 22-Mer ApoA-I analogue and phospholipids complex, elevates HDL-C in atherosclerosis patients. Circulation. 108: 563-564.
34. Miles, J., M. Khan, C. Painchaud, N. Lalwani, S. Drake, and J. Dasseux. 2004. P105 Single-dose Tolerability, Pharmacokinetics, and Cholesterol Mobilization in Hdl-c Fraction Following Intravenous Administration of Etc-642, a 22-mer Apoa-i Analogue and Phospholipids Complex, in Atherosclerosis Patients. Arterioscler. Thromb. Vasc. Biol. J. Am. Hear. Assoc. 24: e-19.
35. Kontush, A., M. Lhomme, and M. J. Chapman. 2013. Unraveling the complexities of the HDL lipidome. J. Lipid Res. 54: 2950-63.
36. Schwendeman, A., D. O. Sviridov, W. Yuan, Y. Guo, E. E. Morin, Y. Yuan, J. Stonik, L. Freeman, A. Ossoli, S. Thacker, S. Killion, M. Pryor, Y. E. Chen, S. Turner, and A. T. Remaley. 2015. The effect of phospholipid composition of reconstituted HDL on its cholesterol efflux and anti-inflammatory properties. J. Lipid Res. 56: 1727-37.
37. Thoolen, B., R. R. Maronpot, T. Harada, A. Nyska, C. Rousseaux, T. Nolte, D. E. Malarkey, W. Kaufmann, K. KOttler, U. Deschl, D. Nakae, R. Gregson, M. P. Vinlove, A. E. Brix, B. Singh, F. Belpoggi, and J. M. Ward. 2010. Proliferative and Nonproliferative Lesions of the Rat and Mouse Hepatobiliary System. Toxicol. Pathol. 38: 5S-81S.
38. Giebeler, A., K. L. Streetz, O. Soehnlein, U. Neumann, J. M. Wang, and L.-O. Brandenburg. 2014. Deficiency of Formyl Peptide Receptor 1 and 2 Is Associated with Increased Inflammation and Enhanced Liver Injury after LPS-Stimulation. PLoS One. 9: e100522.
39. Hamesch, K., E. Borkham-Kamphorst, P. Stmad, and R. Weiskirchen. 2015. Lipopolysaccharide-induced inflammatory liver injury in mice. Lab. Anim. 49: 37-46.
40. de Souza Xavier Costa, N., G. Ribeiro Jnnior, A. A. dos Santos Alemany, L. Belotti, D. H. Zati, M. Frota Cavalcante, M. Matera Veras, S. Ribeiro, E. G. Kallis, P. H. Nascimento Saldiva, M. Dolhnikoff, and L. F. Ferraz da Silva. 2017. Early and late pulmonary effects of nebulized LPS in mice: An acute lung injury model. PLoS One. 12: e0185474.
41. Kim, Y.-H., D.-W. Yoon, J.-H. Kim, J.-H. Lee, and C.-H. Lim. 2014. Effect of remote ischemic post-conditioning on systemic inflammatory response and survival rate in lipopolysaccharide-induced systemic inflammation model. J. Inflamm. 11: 16.
42. Tang, J., D. Li, L. Drake, W. Yuan, S. Deschaine, E. E. Morin, R. Ackermann, K. Olsen, D. E. Smith, and A. Schwendeman. 2016. Influence of route of administration and lipidation of apolipoprotein A-I peptide on pharmacokinetics and cholesterol mobilization. J. Lipid Res. 58: 124-136.
43. Li, D., M. V. Fawaz, E. E. Morin, R. Ming, D. Sviridov, J. Tang, R. Ackermann, K. Olsen, A. T. Remaley, and A. Schwendeman. 2018. Effect of Synthetic High Density Lipoproteins Modification with Polyethylene Glycol on Pharmacokinetics and Pharmacodynamics. Mol. Pharm. 15: 83-96.
44. Boulgaropoulos, B., Z. Arsov, P. Laggner, and G. Pabst. 2011. Stable and unstable lipid domains in ceramide-containing membranes. Biophys. J. 100: 2160-8.
45. Rowe, E. S. 1983. Lipid chain length and temperature dependence of ethanol-phosphatidylcholine interactions. Biochemistry. 22: 3299-3305.
46. Whitmore, M. M., A. Iparraguirre, L. Kubelka, W. Weninger, T. Hai, and B. R. G. Williams. 2007. Negative Regulation of TLR-Signaling Pathways by Activating Transcription Factor-3. J. Immunol. 179: 3622-3630.
47. Gilchrist, M., V. Thorsson, B. Li, A. G. Rust, M. Korb, K. Kennedy, T. Hai, H. Bolouri, and A. Aderem. 2006. Systems biology approaches identify ATF3 as a negative regulator of Toll-like receptor 4. Nature. 441: 173-178.
48. Litman, B. J., E. N. Lewis, and I. W. Levin. 1991. Packing characteristics of highly unsaturated bilayer lipids: Raman spectroscopic studies of multilamellar phosphatidylcholine dispersions. Biochemistry. 30: 313-319.
49. Patel, H., B. Ding, K. Emst, L. Shen, W. Yuan, J. Tang, L. R. Drake, J. Kang, Y. Li, Z. Chen, and A. Schwendeman. 2019. Characterization of apolipoprotein A-I peptide phospholipid interaction and its effect on HDL nanodisc assembly. Int. J. Nanomedicine. 14: 3069-3086.
50. Bonnefont-Rousselot, D., C. Motta, A. O. Khalil, R. Sola, A. E. La Ville, J. Delattre, and M. Gard6s-Albert. 1995. Physicochemical changes in human high-density lipoproteins (HDL) oxidized by gamma radiolysis-generated oxyradicals. Effect on their cholesterol effluxing capacity. Biochim. Biophys. Acta. 1255: 23-30.
51. Girona, J., A. E. LaVille, R. Solh, C. Motta, and L. Masana. 2003. HDL derived from the different phases of conjugated diene formation reduces membrane fluidity and contributes to a decrease in free cholesterol efflux from human THP-1 macrophages. Biochim. Biophys. Acta-Mol. Cell Biol. Lipids. 1633: 143-148.
52. Marmillot, P., S. Patel, and M. R. Lakshman. 2007. Reverse cholesterol transport is regulated by varying fatty acyl chain saturation and sphingomyelin content in reconstituted high-density lipoproteins. Metabolism. 56: 251-9.
53. Ramstedt, B., and J. P. Slotte. 1999. Interaction of cholesterol with sphingomyelins and acyl-chain-matched phosphatidylcholines: a comparative study of the effect of the chain length. Biophys. J. 76: 908-15.
54. Ohvo-Rekila, H., B. Ramstedt, P. Leppimaki, and J. Peter Slotte. 2002. Cholesterol interactions with phospholipids in membranes. Prog. Lipid Res. 41: 66-97.
55. Murphy, A. J., K. J. Woollard, A. Hoang, N. Mukhamedova, R. A. Stirzaker, S. P. A. McCormick, A. T. Remaley, D. Sviridov, and J. Chin-Dusting. 2008. High-Density Lipoprotein Reduces the Human Monocyte Inflammatory Response. Arterioscler. Thromb. Vasc. Biol. 28: 2071-2077.
56. Zhu, X., J.-Y. Y. Lee, J. M. Timmins, J. M. Brown, E. Boudyguina, A. Mulya, A. K. Gebre, M. C. Willingham, E. M. Hiltbold, N. Mishra, N. Maeda, and J. S. Parks. 2008. Increased cellular free cholesterol in macrophage-specific Abcal knock-out mice enhances pro-inflammatory response of macrophages. J. Biol. Chem. 283: 22930-22941.
57. Gilchrist, M., V. Thorsson, B. Li, A. G. Rust, M. Korb, K. Kennedy, T. Hai, H. Bolouri, and A. Aderem. 2006. Systems biology approaches identify ATF3 as a negative regulator of Toll-like receptor 4. Nature. 441: 173-178.
58. Whitmore, M. M., A. Iparraguirre, L. Kubelka, W. Weninger, T. Hai, and B. R. G. Williams. 2007. Negative regulation of TLR-signaling pathways by activating transcription factor-3. J. Immunol. 179: 3622-30.
59. Kwon, J.-W., H.-K. Kwon, H.-J. Shin, Y.-M. Choi, M. A. Anwar, and S. Choi. 2015.
Activating transcription factor 3 represses inflammatory responses by binding to the p65 subunit of NF-κB. Sci. Rep. 5: 14470.
60. Didichenko, S. A., A. V. Navdaev, A. M. O. Cukier, A. Gille, P. Schuetz, M. O. Spycher, P. Thdrond, M. J. Chapman, A. Kontush, and S. D. Wright. 2016. Enhanced HDL Functionality in Small HDL Species Produced Upon Remodeling of HDL by Reconstituted HDL, CSL112. Circ. Res. 119: 751-763.
61. Fawaz, M. V., S. Y. Kim, D. Li, R. Ming, Z. Xia, K. Olsen, I. D. Pogozheva, J. J. G. Tesmerd, and A. Schwendeman. 2019. Phospholipid component defines pharmacokinetic and pharmacodynamic properties of synthetic high-density lipoproteins. J. Pharmacol. Exp. Ther. In press.
62. Schwendeman, A., D. O. Sviridov, W. Yuan, Y. Guo, E. E. Morin, Y. Yuan, J. Stonik, L. Freeman, A. Ossoli, S. Thacker, S. Killion, M. Pryor, Y. E. Chen, S. Turner, and A. T. Remaley. 2015. The effect of phospholipid composition of reconstituted HDL on its cholesterol efflux and anti-inflammatory properties. J. Lipid Res. 56: 1727-37.
63. Wang, Y., X. Zhu, G. Wu, L. Shen, and B. Chen. 2008. Effect of lipid-bound apoA-I cysteine mutants on lipopolysaccharide-induced endotoxemia in mice. J. Lipid Res. 49: 1640-5.

We claim:

1. A method of preventing, attenuating or treating a a condition in a subject, comprising administering to the subject a therapeutically effective amount of a composition comprising a synthetic HDL nanoparticle (sHDL), wherein the sHDL comprises a mixture of at least one HDL apolipoprotein and at least one lipid component, wherein the condition is one or more of sepsis or a sepsis related disorder, conditions and symptoms caused by a viral infection, and conditions and symptoms caused by thrombosis.

2. The method of claim 1, wherein the sepsis related disorder is any condition associated with bacteremia or introduction of lipopolysaccharide into the blood stream or onto an extra-gastrointestinal mucosal surface.

3. The method of claim 1, wherein the sepsis related disorder is a condition selected from endotoxin-related shock, endotoxin-related disseminated intravascular coagulation, endotoxin-related anemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leucopenia resulting from Gram-negative infection, hemodynamic shock and endotoxin-related pyresis.

4. The method of claim 1, wherein for methods of attenuating or treating sepsis or a sepsis related disorder, administration of the composition results in, for example, attenuation of inflammatory activity in the subject through, for example, suppression of NF-kB signaling, regulating TLR4 recruitment into lipid rafts, promoting ATF-3 expression, protecting organs from organ failure, and neutralization of LPS.

5. The method of claim 1, wherein for methods of attenuating or treating sepsis or a sepsis related disorder the composition comprising a sHDL is co-administered with one or more of the following therapeutic agents: alpha-/beta-adrenergic agonists (e.g., norepinephrine, dopamine, dobutamine, epinephrine, vasopressin, phenylephrine), isotonic crystalloids, albumin, antibiotics (e.g., cefotaxime, ticarcillin-clavulanate, piperacillin-tazobactam, imipenem-cilastatin, meropenem, clindamycin, metronidazole, ceftriaxone, ciprofloxacin, cefepime, levofloxacin, vancomycin), and corticosteroids (e.g., hydrocortisone, dexamethasone).

6. The method of claim 1, wherein the conditions and symptoms caused by a viral infection is any infection related to COVID-19, influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus.

7. The method of claim 1, wherein for methods of attenuating or treating a viral infection, administration of the composition results in, for example, modulation of lipid raft composition resulting in reduced levels ACE2 and SARS-COV-2 virus cell entry; inhibition of SARS-COV2 S protein induced NF-kB activation and reduction of proinflammatory cytokine release by immune effector cells; and inhibiting endothelial activation and dysfunction.

8. The method of claim 1, wherein for methods of attenuating or treating a viral infection the composition comprising a sHDL is co-administered with one or more of the following therapeutic agents: remdesivir, dexamethasone, and hydroxychloroquine.

9. The method of claim 1, the conditions and symptoms caused by thrombosis are related to a venous thrombosis or an arterial thrombosis.

10. The method of claim 1, wherein for methods of attenuating or treating conditions and symptoms caused by thrombosis, administration of the composition results in, for example, reduction of platelet activity, prevention of thrombus formation, and reduction of platelet aggregation.

11. The method of claim 1, wherein for methods of attenuating or treating conditions and symptoms caused by thrombosis the composition comprising a sHDL is co-administered with one or more of the following therapeutic agents: heparin; tPA; anistreplase; streptokinase; urokinase; a coumadin; warfarin; idraparinux; fondaparinux; aspririn; an adenosine diphosphate receptor inhibitor; a phosphodiesterase inhibitor; a glycoprotein IIB/IIA inhibitor; an adenosine reuptake inhibitor; and a thromboxane receptor antagonist.

12. The method of claim 1, wherein the administering to the subject a therapeutically effective amount of a composition comprising a sHDL comprises a continuous infusion of sHDL and/or non-continuous infusions of sHDL.

13. The method of claim 1,
wherein the at least one HDL apolipoprotein is an ApoA-I mimetic represented by SEQ ID NO: 4 (22A), and
wherein the at least one lipid component is selected from dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), and 1,2-distearoyl-sn-glycero-3-phosphatidylcholine (DSPC).

14. The method of claim 1, wherein the subject is a human being suffering from or at risk of suffering from one or more of conditions and symptoms caused by sepsis or a sepsis related disorder, conditions and symptoms caused by a viral infection, and conditions and symptoms caused by thrombosis.

15. The method of claim 1, wherein the average particle size of the sHDL nanoparticle is at or between 6-20 nm.

16. The method of claim 1, wherein the average particle size of the sHDL nanoparticle is at or between 7-12 nm.

17. The method of claim 1, wherein the molar ratio of the HDL apolipoprotein component to the lipid component is about 2:1 to 200:1.

18. The method of claim 1, wherein the lipid component comprises a combination of one or any combination of sphingomyelin (SM), D-erythrose-sphingomyelin, D-erythrose dihydrosphingomyelin, palmitoylsphingomyelin, lysophospholipids, galactocerebroside, gangliosides, cerebrosides, glycerides, triglycerides, diglycerides, small alkyl chain phospholipids, phosphatidylcholine, egg phosphatidylcholine, soybean phosphatidylcholine, dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), 1,2-distearoyl-sn-glycero-3-phosphatidylcholine (DSPC), distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, ceramides, a phosphatidylserine, dimyristoylphosphatidylserine, dipalmitoylphosphatidylserine, brain phosphatidylserine, brain sphingomyelin, egg sphingomyelin, milk sphingomyelin, palmitoyl sphingomyelin, phytosphingomyelin, dipalmitoylsphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives, lyso-phosphotydyl choline, lyso-sphingomyelin, dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], Lyso phoshphatidic acid, Lyso phosphatidylcholine, OA-NO₂ (nitrated oleic acid 9- and 10-nitro-cis-octedecenolic acids), LNO₂ (nitrated linoleic Acid 9-, 10-, 12- and 13-nitro-cis-octedecadienoic acids), AA-NO₂ (nitrated Arachidonic Acid 5-, 6-, 8-, 9-, 11-, 12-, 14,- and 15-nitro-cis-eicosatetraenoic acids), CLNO₂ (nitrated cholesteryl linoleate cholestaryl-9-, 10-, 12- and 13-nitro-cis-octedecadiencates), fatty acid, omega-3 polyunsaturated fatty acids, hexadecatrienoic acid (HTA; 16:3 (n-3); all-cis-7,10,13-hexadecatrienoic acid), α-Linolenic acid (ALA; 18:3 (n-3); all-cis-9,12,15-octadecatrienoic acid), stearidonic acid (SDA; 18:4 (n-3); all-cis-6,9,12,15-octadecatetraenoic acid), eicosatrienoic acid (ETE; 20:3 (n-3); all-cis-11,14,17-eicosatrienoic acid), eicosatetraenoic acid (ETA; 20:4 (n-3); all-cis-8,11,14,17-eicosatetraenoic acid), eicosapentaenoic acid (EPA; 20:5 (n-3); all-cis-5,8,11,14,17-eicosapentaenoic acid), heneicosapentaenoic acid (HPA; 21:5 (n-3); all-cis-6,9,12,15,18-heneicosapentaenoic acid): docosapentaenoic acid (DPA; clupanodonic acid; 22:5 (n-3); all-cis-7,10,13,16,19-docosapentaenoic acid), docosahexaenoic acid (DHA; 22:6 (n-3); all-cis-4,7,10,13,16,19-docosahexaenoic acid), tetracosapentaenoic acid; 24:5 (n-3); all-cis-9,12,15,18,21-tetracosapentaenoic acid), tetracosahexaenoic acid (Nisinic acid; 24:6 (n-3), all-cis-6,9,12,15,18,21-tetracosahexaenoic acid), sphingosine-1-phosphate analogs, sphingosine-1-phosphate antagonists, sphingosine-1-phosphate agonists, sphingosine-1-phosphate receptor agonists, sphingosine-1-phosphate receptor antagonists, and sphingosine-1-phosphate receptor analogs.

19. The method of claim 1, wherein the lipid component comprises neutral phospholipids, negatively charged phospholipids, positively charged phospholipids, or a combination thereof.

20. The method of claim 19, wherein fatty acid chains on the phospholipids are preferably from 12 to 26 or 16 to 26 carbons in length and can vary in degree of saturation from saturated to mono-unsaturated.

21. The method of claim 1, wherein the HDL apolipoprotein component is selected from the group consisting of apolipoprotein A-I (apo A-I), apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

22. The method of claim 21, wherein the ApoA-I mimetic is described by any of SEQ ID NOs: 1-336 and SEQ ID NOs: 337-371.

23. The method of claim 1, wherein the ratio of HDL apolipoprotein component to lipid component is at or between 1:1 to 1:4 wt/wt.

24. The method of claim 1, wherein the ratio of HDL apolipoprotein component to lipid component is at or between 1:1.5 to 1:3 wt/wt.

25. The method of claim 1, wherein the ratio of HDL apolipoprotein component to lipid component is 1:2 wt/wt.

26. The method of claim 1, wherein the sHDL nanoparticle has less than 5% free lipid component impurity.

27. The method of claim 1, wherein the sHDL nanoparticle has less than 20% free HDL apolipoprotein component impurity.

28. The method of claim 18, wherein approximately 25% of the lipid component is cholesterol and/or cholesterol ester.

29. The method of claim 18, wherein approximately 10% of the lipid component is cholesterol and/or cholesterol ester.

30. The method of claim 18, wherein approximately 5% of the lipid component is cholesterol and/or cholesterol ester.

31. The method of claim 18, wherein approximately 1% of the lipid component is cholesterol and/or cholesterol ester.

32. The method of claim 1, wherein composition contains no more than 1 endotoxin unit (EU), no more than 0.5 EU, no more than 0.3 EU or no more than 0.1 EU of endotoxin per milligram of HDL apolipoprotein component.

33. The method of claim 1, wherein the composition comprising sHDL is at least 90%, at least 92.5%, at least 95%, at least 96%, at least 97% or at least 98% pure.

34. The method of claim 1, wherein the composition comprising sHDL is at least 80%, at least 85%, at least 90% or at least 95% homogeneous, as reflected by a single peak in gel permeation chromatography.

35. The method of claim 1, wherein at least 80%, at least 85%, at least 90% or at least 95% of the sHDL nanoparticles range 4 nm to 12 nm in size, 6 nm to 12 nm in size, or 8 nm to 12 nm in size, as measured by GPC or DLS.

36. The method of claim 1, wherein at least 95%, at least 96%, at least 97%, at least 98% or at least 99% of the HDL apolipoprotein component is in complexes.

37. A composition comprising a synthetic HDL nanoparticle (sHDL) for preventing, attenuating, and/or treating a condition in a subject, wherein the sHDL comprises a mixture of at least one HDL apolipoprotein component and at least one lipid component, wherein the condition is one or more of sepsis or a sepsis related disorder, conditions and symptoms caused by a viral infection, and conditions and symptoms caused by thrombosis.

38. The composition of claim 37, wherein the sepsis related disorder is any condition associated with bacteremia or introduction of lipopolysaccharide into the blood stream or onto an extra-gastrointestinal mucosal surface.

39. The composition of claim 37, wherein the sepsis related disorder is a condition selected from endotoxin-related shock, endotoxin-related disseminated intravascular coagulation, endotoxin-related anemia, endotoxin-related thrombocytopenia, endotoxin-related adult respiratory distress syndrome, endotoxin-related renal failure, endotoxin-related liver disease or hepatitis, systemic immune response syndrome (SIRS) resulting from Gram-negative infection, Gram-negative neonatal sepsis, Gram-negative meningitis, Gram-negative pneumonia, neutropenia and/or leucopenia resulting from Gram-negative infection, hemodynamic shock and endotoxin-related pyresis.

40. The composition of claim 37, wherein the conditions and symptoms caused by a viral infection is any infection related to COVID-19, influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus.

41. The composition of claim 37,
wherein the at least one HDL apolipoprotein is an ApoA-I mimetic represented by SEQ ID NO: 4 (22A), and
wherein the at least one lipid component is selected from dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), and 1,2-distearoyl-sn-glycero-3-phosphatidylcholine (DSPC).

42. The composition of claim 37, wherein the average particle size of the sHDL nanoparticle is at or between 6-20 nm.

43. The composition of claim 37, wherein the average particle size of the sHDL nanoparticle is at or between 7-12 nm.

44. The composition of claim 37, wherein the molar ratio of the HDL apolipoprotein component to the lipid component is about 2:1 to 200:1.

45. The composition of claim 37, wherein the lipid component comprises a combination of sphingomyelin (SM) and phospholipid.

46. The composition of claim 37, wherein the molar ratio of the HDL apolipoprotein component to the lipid component is about 2:1 to 200:1.

47. The composition of claim 37, wherein the lipid component comprises a combination of one or any combination of sphingomyelin (SM), D-erythrose-sphingomyelin, D-erythrose dihydrosphingomyelin, palmitoylsphingomyelin, lysophospholipids, galactocerebroside, gangliosides, cerebrosides, glycerides, triglycerides, diglycerides, small alkyl chain phospholipids, phosphatidylcholine, egg phosphatidylcholine, soybean phosphatidylcholine, dipalmitoylphosphatidylcholine (DPPC), dimyristoylphosphatidylcholine, 1-palmitoyl-2-oleoyl-phosphatidylcholine (POPC), 1,2-dimyristoyl-sn-glycero-3-phosphatidylcholine (DMPC), 1,2-distearoyl-sn-glycero-3-phosphatidylcholine (DSPC), distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, ceramides, a phosphatidylserine, dimyristoylphosphatidylserine, dipalmitoylphosphatidylserine, brain phosphatidylserine, brain sphingomyelin, egg sphingomyelin, milk sphingomyelin, palmitoyl sphingomyelin, phytosphingomyelin, dipalmitoylsphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives, lyso-phosphotydyl choline, lyso-sphingomyelin, dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], Lyso phoshphatidic acid, Lyso phosphatidylcholine, OA-NO₂ (nitrated oleic acid 9- and 10-nitro-cis-octedecenolic acids), LNO₂ (nitrated linoleic Acid 9-, 10-, 12- and 13-nitro-cis-octedecadienoic acids), AA-NO₂ (nitrated Arachidonic Acid 5-, 6-, 8-, 9-, 11-, 12-, 14,- and 15-nitro-cis-eicosatetraenoic acids), CLNO₂ (nitrated cholesteryl linoleate cholestaryl-9-, 10-, 12- and 13-nitro-cis-octedecadiencates), fatty acid, omega-3 polyunsaturated fatty acids, hexadecatrienoic acid (HTA; 16:3 (n-3); all-cis-7,10,13-hexadecatrienoic acid), a-Linolenic acid (ALA; 18:3 (n-3); all-cis-9,12,15-octadecatrienoic acid), stearidonic acid (SDA; 18:4 (n-3); all-cis-6,9,12,15-octadecatetraenoic acid), eicosatrienoic acid (ETE; 20:3 (n-3); all-cis-11,14,17-eicosatrienoic acid), eicosatetraenoic acid (ETA; 20:4 (n-3); all-cis-8,11,14,17-eicosatetraenoic acid), eicosapentaenoic acid (EPA; 20:5 (n-3); all-cis-5,8,11,14,17-eicosapentaenoic acid), heneicosapentaenoic acid (HPA; 21:5 (n-3); all-cis-6,9,12,15,18-heneicosapentaenoic acid); docosapentaenoic acid (DPA; clupanodonic acid; 22:5 (n-3); all-cis-7,10,13,16,19-docosapentaenoic acid), docosahexaenoic acid (DHA; 22:6 (n-3); all-cis-4,7,10,13,16,19-docosahexaenoic acid), tetracosapentaenoic acid; 24:5 (n-3); all-cis-9,12,15,18,21-tetracosapentaenoic acid), tetracosahexaenoic acid (Nisinic acid; 24:6 (n-3), all-cis-6,9,12,15,18,21-tetracosahexaenoic acid), sphingosine-1-phosphate analogs, sphingosine-1-phosphate antagonists, sphingosine-1-phosphate agonists, sphingosine-1-phosphate receptor agonists, sphingosine-1-phosphate receptor antagonists, and sphingosine-1-phosphate receptor analogs.

48. The composition of claim 37, wherein the lipid component comprises neutral phospholipids, negatively charged phospholipids, positively charged phospholipids, or a combination thereof.

49. The composition of claim 78, wherein the fatty acid chains on the phospholipids are preferably from 12 to 26 or 16 to 26 carbons in length and can vary in degree of saturation from saturated to mono-unsaturated.

50. The composition of claim 37, wherein the HDL apolipoprotein component is selected from the group consisting of apolipoprotein A-I (apo A-I), apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

51. The composition of claim 80, wherein the ApoA-I mimetic is described by any of SEQ ID NOs: 1-336 and SEQ ID NOs: 337-371.

52. The composition of claim 37, wherein the ratio of HDL apolipoprotein component to lipid component is at or between 1:1 to 1:4 wt/wt.

53. The composition of claim 37, wherein the ratio of HDL apolipoprotein component to lipid component is at or between 1:1.5 to 1:3 wt/wt.

54. The composition of claim 37, wherein the ratio of HDL apolipoprotein component to lipid component is 1:2 wt/wt.

55. The composition of claim 37, wherein the sHDL nanoparticle has less than 5% free lipid component impurity.

56. The composition of claim 37, wherein the sHDL nanoparticle has less than 20% free HDL apolipoprotein impurity.

57. The composition of claim 47, wherein approximately 25% of the lipid component is cholesterol and/or cholesterol ester.

58. The composition of claim 47, wherein approximately 10% of the lipid component is cholesterol and/or cholesterol ester.

59. The composition of claim 47, wherein approximately 5% of the lipid component is cholesterol and/or cholesterol ester.

60. The composition of claim 47, wherein approximately 1% of the lipid component is cholesterol and/or cholesterol ester.

61. The composition of claim 37, wherein the composition further comprises an anti-inflammatory agent.

62. The composition of claim 37, wherein composition contains no more than 1 endotoxin unit (EU), no more than 0.5 EU, no more than 0.3 EU or no more than 0.1 EU of endotoxin per milligram of HDL apolipoprotein component.

63. The composition of claim 37, wherein the composition comprising sHDL is at least 90%, at least 92.5%, at least 95%, at least 96%, at least 97% or at least 98% pure.

64. The composition of claim 37, wherein the composition comprising sHDL is at least 80%, at least 85%, at least 90% or at least 95% homogeneous, as reflected by a single peak in gel permeation chromatography.

65. The composition of claim 37, wherein at least 80%, at least 85%, at least 90% or at least 95% of the sHDL nanoparticles range 4 nm to 12 nm in size, 6 nm to 12 nm in size, or 8 nm to 12 nm in size, as measured by GPC or DLS.

66. The composition of claim 37, wherein at least 95%, at least 96%, at least 97%, at least 98% or at least 99% of the HDL apolipoprotein component is in complexes.

67. A method for treating, ameliorating and/or preventing symptoms related to viral infection in a subject, comprising administering to the subject a composition of claim 37.

68. The method of claim 67, wherein the symptoms related to viral infection in a subject are one or more of fever, fatigue, dry cough, myalgias, dyspnea, acute respiratory distress syndrome, and pneumonia.

69. The method of claim 67, wherein the subject is a human subject suffering from or at risk of suffering from a condition related to SARS-CoV-2 infection (e.g., COVID-19).

70. The method of claim 67, wherein the pharmaceutical composition is dispersed in a pharmaceutically acceptable carrier.

71. The method of claim 67, wherein the administering is oral, intravenous, or topical.

72. The method of claim 67, further comprising administering to the subject remdesivir, dexamethasone, and/or hydroxychloroquine.

73. A method for treating, ameliorating and/or preventing acute respiratory distress syndrome in a subject, comprising administering to the subject a composition of claim 37.

74. The method of claim 73, wherein the acute respiratory distress syndrome is related to SARS-CoV-2 infection (e.g., COVID-19).

75. The method of claim 73, wherein the subject is a human subject suffering from or at risk of suffering from a condition related to SARS-CoV-2 infection (e.g., COVID-19).

76. The method of claim 73, wherein the administering is oral, intravenous or topical.

77. The method of claim 73, further comprising administering to the subject remdesivir, dexamethasone, and/or hydroxychloroquine.

78. A method for treating, ameliorating and/or preventing pneumonia in a subject, comprising administering to the subject a composition of claim 37.

79. The method of claim 78, wherein the pneumonia is related to SARS-CoV-2 infection (e.g., COVID-19).

80. The method of claim 78, wherein the subject is a human subject suffering from or at risk of suffering from a condition related to SARS-CoV-2 infection (e.g., COVID-19).

81. The method of claim 78, wherein the administering is oral, intravenous or topical.

82. The method of claim 78, further comprising administering an additional agent for treating pneumonia.

83. The method of claim 78, further comprising administering to the subject remdesivir, dexamethasone, and/or hydroxychloroquine.


SEQUENCE LISTING (371 sequences)

SEQ ID NO: 1 - protein, 22 aa, Artificial sequence
  synthetic
PVLDLFRELLNELLEZLKQKLK

SEQ ID NO: 2 - protein, 23 aa, Artificial sequence
  synthetic
GVLDLFRELLNELLEALKQKLKK

SEQ ID NO: 3 - protein, 22 aa, Artificial sequence
  synthetic
PVLDLFRELLNELLEWLKQKLK

SEQ ID NO: 4 - protein, 22 aa, Artificial sequence
  synthetic
PVLDLFRELLNELLEALKQKLK

SEQ ID NO: 5 - protein, 23 aa, Artificial sequence
  synthetic
PVLDLFRELLNELLEALKQKLKK

SEQ ID NO: 6 - protein, 22 aa, Artificial sequence
  synthetic
  misc_feature: Xaa = Aib
  misc_feature (13)..(13): Xaa can be any naturally occurring amino acid
PVLDLFRELLNEXLEALKQKLK

SEQ ID NO: 7 - protein, 22 aa, Artificial sequence
  synthetic
PVLDLFKELLNELLEALKQKLK

SEQ ID NO: 8 - protein, 22 aa, Artificial sequence
  synthetic
PVLDLFRELLNEGLEALKQKLK

SEQ ID NO: 9 - protein, 22 aa, Artificial sequence
  synthetic
PVLDLFRELGNELLEALKQKLK

SEQ ID NO: 10 - protein, 22 aa, Artificial sequence
  synthetic
PVLDLFRELLNELLEAZKQKLK

SEQ ID NO: 11 - protein, 22 aa, Artificial sequence
  synthetic
PVLDLFKELLQELLEALKQKLK

SEQ ID NO: 12 - protein, 22 aa, Artificial sequence
  synthetic
PVLDLFRELLNELLEAGKQKLK

SEQ ID NO: 13 - protein, 22 aa, Artificial sequence
  synthetic
GVLDLFRELLNEGLEALKQKLK

SEQ ID NO: 14 - protein, 22 aa, Artificial sequence
  synthetic
  misc_feature: Xaa = Orn
  misc_feature (18)..(18): Xaa can be any naturally occurring amino acid
  misc_feature (20)..(20): Xaa can be any naturally occurring amino acid
  misc_feature (22)..(22): Xaa can be any naturally occurring amino acid
PVLDLFRELLNELLEALXQXLX

SEQ ID NO: 15 - protein, 22 aa, Artificial sequence
  synthetic
PVLDLFRELWNELLEALKQKLK

SEQ ID NO: 16 - protein, 22 aa, Artificial sequence
  synthetic
PVLDLLRELLNELLEALKQKLK

SEQ ID NO: 17 - protein, 22 aa, Artificial sequence
  synthetic
PVLELFKELLQELLEALKQKLK

SEQ ID NO: 18 - protein, 22 aa, Artificial sequence
  synthetic
GVLDLFRELLNELLEALKQKLK

SEQ ID NO: 19 - protein, 22 aa, Artificial sequence
  synthetic
PVLDLFRELLNEGLEALKQKLK

SEQ ID NO: 20 - protein, 22 aa, Artificial sequence
  synthetic
PVLDLFREGLNELLEALKQKLK

SEQ ID NO: 21 - protein, 22 aa, Artificial sequence
  synthetic
PVLDLFRELLNELLEALKQKLK

SEQ ID NO: 22 - protein, 22 aa, Artificial sequence
  synthetic
PVLDLFRELLNELLEGLKQKLK

SEQ ID NO: 23 - protein, 22 aa, Artificial sequence
  synthetic
PLLELFKELLQELLEALKQKLK

SEQ ID NO: 24 - protein, 22 aa, Artificial sequence
  synthetic
PVLDLFRELLNELLEALQKKLK

SEQ ID NO: 25 - protein, 22 aa, Artificial sequence
  synthetic
  misc_feature: Xaa = Aib
  misc_feature (13)..(13): Xaa can be any naturally occurring amino acid
PVLDFFRELLNEXLEALKQKLK

SEQ ID NO: 26 - protein, 22 aa, Artificial sequence
  synthetic
PVLDLFRELLNELLELLKQKLK

SEQ ID NO: 27 - protein, 22 aa, Artificial sequence
  synthetic
PVLDLFRELLNELZEALKQKLK

SEQ ID NO: 28 - protein, 22 aa, Artificial sequence
  synthetic
PVLDLFRELLNELWEALKQKLK

SEQ ID NO: 29 - protein, 22 aa, Artificial sequence
  synthetic
AVLDLFRELLNELLEALKQKLK

SEQ ID NO: 30 - protein, 22 aa, Artificial sequence
  synthetic
PVLDLPRELLNELLEALKQKLK

SEQ ID NO: 31 - protein, 22 aa, Artificial sequence
  synthetic
  misc_feature: Xaa = Aib
  misc_feature (13)..(13): Xaa can be any naturally occurring amino acid
PVLDLFLELLNEXLEALKQKLK

SEQ ID NO: 32 - protein, 22 aa, Artificial sequence
  synthetic
  misc_feature: Xaa = Aib
  misc_feature (1)..(1): Xaa can be any naturally occurring amino acid
XVLDLFRELLNELLEALKQKLK

SEQ ID NO: 33 - protein, 22 aa, Artificial sequence
  synthetic
PVLDLFREKLNELLEALKQKLK

SEQ ID NO: 34 - protein, 22 aa, Artificial sequence
  synthetic
PVLDZFRELLNELLEALKQKLK

SEQ ID NO: 35 - protein, 22 aa, Artificial sequence
  synthetic
PVLDWFRELLNELLEALKQKLK

SEQ ID NO: 36 - protein, 22 aa, Artificial sequence
  synthetic
PLLELLKELLQELLEALKQKLK

SEQ ID NO: 37 - protein, 22 aa, Artificial sequence
  synthetic
PVLDLFREWLNELLEALKQKLK

SEQ ID NO: 38 - protein, 22 aa, Artificial sequence
  synthetic
  misc_feature: Xaa = Aib
  misc_feature (13)..(13): Xaa can be any naturally occurring amino acid
PVLDLFRELLNEXLEAWKQKLK

SEQ ID NO: 39 - protein, 22 aa, Artificial sequence
  synthetic
PVLDLFRELLEELLKALKKKLK

SEQ ID NO: 40 - protein, 22 aa, Artificial sequence
  synthetic
PVLDLFNELLRELLEALQKKLK

SEQ ID NO: 41 - protein, 22 aa, Artificial sequence
  synthetic
  misc_feature: Xaa = Aib
  misc_feature (13)..(13): Xaa can be any naturally occurring amino acid
PVLDLWRELLNEXLEALKQKLK

SEQ ID NO: 42 - protein, 22 aa, Artificial sequence
  synthetic
  misc_feature: Xaa = Aib
  misc_feature (13)..(13): Xaa can be any naturally occurring amino acid
PVLDEFREKLNEXWEALKQKLK

SEQ ID NO: 43 - protein, 22 aa, Artificial sequence
  synthetic
  misc_feature: Xaa = Aib
  misc_feature (13)..(13): Xaa can be any naturally occurring amino acid
PVLDEFREKLWEXLEALKQKLK

SEQ ID NO: 44 - protein, 22 aa, Artificial sequence
  synthetic
  misc_feature: Xaa = Aib
  misc_feature (13)..(13): Xaa can be any naturally occurring amino acid
PVLDEFREKLNEXLEALKQKLK

SEQ ID NO: 45 - protein, 22 aa, Artificial sequence
  synthetic
  misc_feature: Xaa = Aib
  misc_feature (13)..(13): Xaa can be any naturally occurring amino acid
PVLDEFREKLNEXLEALKQKLK

SEQ ID NO: 46 - protein, 22 aa, Artificial sequence
  synthetic
  misc_feature: Xaa = Aib
  misc_feature (13)..(13): Xaa can be any naturally occurring amino acid
PVLDLFREKLNEXLEALKQKLK

SEQ ID NO: 47 - protein, 21 aa, Artificial sequence
  synthetic
VLDLFRELLNEGLEALKQKLK

SEQ ID NO: 48 - protein, 22 aa, Artificial sequence
  synthetic
PVLDLFRELLNELLEALKQKLK

SEQ ID NO: 49 - protein, 22 aa, Artificial sequence
  synthetic
PVLDLFRNLLEKLLEALEQKLK

SEQ ID NO: 50 - protein, 22 aa, Artificial sequence
  synthetic
  misc_feature: Xaa = Aib
  misc_feature (13)..(13): Xaa can be any naturally occurring amino acid
PVLDLFRELLWEXLEALKQKLK

SEQ ID NO: 51 - protein, 22 aa, Artificial sequence
  synthetic
  misc_feature: Xaa = Aib
  misc_feature (13)..(13): Xaa can be any naturally occurring amino acid
PVLDLFWELLNEXLEALKQKLK

SEQ ID NO: 52 - protein, 22 aa, Artificial sequence
  synthetic
  misc_feature: Xaa = Aib
  misc_feature (13)..(13): Xaa can be any naturally occurring amino acid
PVWDEFREKLNEXLEALKQKLK

SEQ ID NO: 53 - protein, 22 aa, Artificial sequence
  synthetic
VVLDLFRELLNELLEALKQKLK

SEQ ID NO: 54 - protein, 22 aa, Artificial sequence
  synthetic
PVLDLFRELLNEWLEALKQKLK

SEQ ID NO: 55 - protein, 19 aa, Artificial sequence
  synthetic
PLFRELLNELLEALKQKLK

SEQ ID NO: 56 - protein, 22 aa, Artificial sequence
  synthetic
PVLDLFRELLNELLEALKQKKK

SEQ ID NO: 57 - protein, 22 aa, Artificial sequence
  synthetic
PVLDLFRNLLEELLKALEQKLK

SEQ ID NO: 58 - protein, 21 aa, Artificial sequence
  synthetic
  misc_feature: Xaa = Aib
  misc_feature (13)..(13): Xaa can be any naturally occurring amino acid
PVLDEFREKLNEXLEALKQKL

SEQ ID NO: 59 - protein, 22 aa, Artificial sequence
  synthetic
LVLDLFRELLNELLEALKQKLK

SEQ ID NO: 60 - protein, 19 aa, Artificial sequence
  synthetic
PVLDLFRELLNELLEALKQ

SEQ ID NO: 61 - protein, 22 aa, Artificial sequence
  synthetic
  misc_feature: Xaa = Aib
  misc_feature (13)..(13): Xaa can be any naturally occurring amino acid
PVLDEFRWKLNEXLEALKQKLK

SEQ ID NO: 62 - protein, 22 aa, Artificial sequence
  synthetic
  misc_feature: Xaa = Aib
  misc_feature (13)..(13): Xaa can be any naturally occurring amino acid
PVLDEWREKLNEXLEALKQKLK

SEQ ID NO: 63 - protein, 22 aa, Artificial sequence
  synthetic
  misc_feature: Xaa = Aib
  misc_feature (13)..(13): Xaa can be any naturally occurring amino acid
PVLDFFREKLNEXLEALKQKLK

SEQ ID NO: 64 - protein, 22 aa, Artificial sequence
  synthetic
  misc_feature: Xaa = Aib
  misc_feature (13)..(13): Xaa can be any naturally occurring amino acid
PWLDEFREKLNEXLEALKQKLK

SEQ ID NO: 65 - protein, 21 aa, Artificial sequence
  synthetic
  misc_feature: Xaa = Aib
  misc_feature (12)..(12): Xaa can be any naturally occurring amino acid
VLDEFREKLNEXLEALKQKLK

SEQ ID NO: 66 - protein, 22 aa, Artificial sequence
  synthetic
PVLDLFRNLLEELLEALQKKLK

SEQ ID NO: 67 - protein, 21 aa, Artificial sequence
  synthetic
VLDLFRELLNELLEALKQKLK

SEQ ID NO: 68 - protein, 22 aa, Artificial sequence
  synthetic
  misc_feature: Xaa = Aib
  misc_feature (13)..(13): Xaa can be any naturally occurring amino acid
PVLDEFRELLKEXLEALKQKLK

SEQ ID NO: 69 - protein, 22 aa, Artificial sequence
  synthetic
  misc_feature: Xaa = Aib
  misc_feature (13)..(13): Xaa can be any naturally occurring amino acid
PVLDEFRKKLNEXLEALKQKLK

SEQ ID NO: 70 - protein, 22 aa, Artificial sequence
  synthetic
  misc_feature: Xaa = Aib
  misc_feature (13)..(13): Xaa can be any naturally occurring amino acid
PVLDEFRELLYEXLEALKQKLK

SEQ ID NO: 71 - protein, 22 aa, Artificial sequence
  synthetic
  misc_feature: Xaa = Aib
  misc_feature (14)..(14): Xaa can be any naturally occurring amino acid
PVLDEFREKLNELXEALKQKLK

SEQ ID NO: 72 - protein, 22 aa, Artificial sequence
  synthetic
  misc_feature: Xaa = Aib
  misc_feature (13)..(13): Xaa can be any naturally occurring amino acid
PVLDLFRELLNEXLWALKQKLK

SEQ ID NO: 73 - protein, 22 aa, Artificial sequence
  synthetic
  misc_feature: Xaa = Aib
  misc_feature (13)..(13): Xaa can be any naturally occurring amino acid
PVLDEFWEKLNEXLEALKQKLK

SEQ ID NO: 74 - protein, 22 aa, Artificial sequence
  synthetic
  misc_feature: Xaa = Aib
  misc_feature (13)..(13): Xaa can be any naturally occurring amino acid
PVLDKFREKLNEXLEALKQKLK

SEQ ID NO: 75 - protein, 22 aa, Artificial sequence
  synthetic
PVLDEFREKLNEELEALKQKLK

SEQ ID NO: 76 - protein, 22 aa, Artificial sequence
  synthetic
  misc_feature: Xaa = Aib
  misc_feature (13)..(13): Xaa can be any naturally occurring amino acid
PVLDEFRELLFEXLEALKQKLK

SEQ ID NO: 77 - protein, 22 aa, Artificial sequence
  synthetic
  misc_feature: Xaa = Aib
  misc_feature (13)..(13): Xaa can be any naturally occurring amino acid
PVLDEFREKLNKXLEALKQKLK

SEQ ID NO: 78 - protein, 22 aa, Artificial sequence
  synthetic
  misc_feature (13)..(13): Xaa can be any naturally occurring amino acid
PVLDEFRDKLNEXLEALKQKLK

SEQ ID NO: 79 - protein, 22 aa, Artificial sequence
  synthetic
PVLDEFRELLNELLEALKQKLK

SEQ ID NO: 80 - protein, 22 aa, Artificial sequence
  synthetic
PVLDLFERLLNELLEALQKKLK

SEQ ID NO: 81 - protein, 22 aa, Artificial sequence
  synthetic
  misc_feature: Xaa = Aib
  misc_feature (13)..(13): Xaa can be any naturally occurring amino acid
PVLDEFREKLNWXLEALKQKLK

SEQ ID NO: 82 - protein, 20 aa, Artificial sequence
  synthetic
  misc_feature: Xaa = Aib
  misc_feature (11)..(11): Xaa can be any naturally occurring amino acid
LDEFREKLNEXLEALKQKLK

SEQ ID NO: 83 - protein, 22 aa, Artificial sequence
  synthetic
  misc_feature: Xaa = Aib
  misc_feature (13)..(13): Xaa can be any naturally occurring amino acid
PVLDEFREKLNEXLEALWQKLK

SEQ ID NO: 84 - protein, 22 aa, Artificial sequence
  synthetic
PVLDEFREKLNELLEALKQKLK

SEQ ID NO: 85 - protein, 21 aa, Artificial sequence
  synthetic
PLDLFRELLNELLEALKQKLK

SEQ ID NO: 86 - protein, 22 aa, Artificial sequence
  synthetic
PVLELFERLLDELLNALQKKLK

SEQ ID NO: 87 - protein, 22 aa, Artificial sequence
  synthetic
PLLELLKELLQELLEALKQKLK

SEQ ID NO: 88 - protein, 22 aa, Artificial sequence
  synthetic
  misc_feature: Xaa = Aib
  misc_feature (13)..(13): Xaa can be any naturally occurring amino acid
PVLDKFRELLNEXLEALKQKLK

SEQ ID NO: 89 - protein, 22 aa, Artificial sequence
  synthetic
  misc_feature: Xaa = Aib
  misc_feature (13)..(13): Xaa can be any naturally occurring amino acid
PVLDEFREKLNEXLWALKQKLK

SEQ ID NO: 90 - protein, 19 aa, Artificial sequence
  synthetic
  misc_feature: Xaa = Aib
  misc_feature (10)..(10): Xaa can be any naturally occurring amino acid
DEFREKLNEXLEALKQKLK

SEQ ID NO: 91 - protein, 22 aa, Artificial sequence
  synthetic
  misc_feature: Xaa = Aib
  misc_feature (13)..(13): Xaa can be any naturally occurring amino acid
PVLDEFRELLNEXLEALKQKLK

SEQ ID NO: 92 - protein, 22 aa, Artificial sequence
  synthetic
  misc_feature: Xaa = Aib
  misc_feature (13)..(13): Xaa can be any naturally occurring amino acid
PVLDEFRELYNEXLEALKQKLK

SEQ ID NO: 93 - protein, 22 aa, Artificial sequence
  synthetic
  misc_feature: Xaa = Aib
  misc_feature (13)..(13): Xaa can be any naturally occurring amino acid
PVLDEFREKLNEXLKALKQKLK

SEQ ID NO: 94 - protein, 22 aa, Artificial sequence
  synthetic
PVLDEFREKLNEALEALKQKLK

SEQ ID NO: 95 - protein, 22 aa, Artificial sequence
  synthetic
  misc_feature: Xaa = Aib
  misc_feature (13)..(13): Xaa can be any naturally occurring amino acid
PVLDLFRELLNLXLEALKQKLK

SEQ ID NO: 96 - protein, 22 aa, Artificial sequence
  synthetic
  misc_feature: Xaa = Aib
  misc_feature (13)..(13): Xaa can be any naturally occurring amino acid
PVLDLFRELLNEXLEALKQKLK

SEQ ID NO: 97 - protein, 15 aa, Artificial sequence
  synthetic
PVLDLFRELLNELLE

SEQ ID NO: 98 - protein, 22 aa, Artificial sequence
  synthetic
PVLDLFRELLNEELEALKQKLK

SEQ ID NO: 99 - protein, 22 aa, Artificial sequence
  synthetic
KLKQKLAELLENLLERFLDLVP

SEQ ID NO: 100 - protein, 22 aa, Artificial sequence
  synthetic
PVLDLFRELLNELLEALKQKLK

SEQ ID NO: 101 - protein, 22 aa, Artificial sequence
  synthetic
  misc_feature: Xaa = Aib
  misc_feature (13)..(13): Xaa can be any naturally occurring amino acid
PVLDLFRELLNWXLEALKQKLK

SEQ ID NO: 102 - protein, 22 aa, Artificial sequence
  synthetic
  misc_feature: Xaa = Aib
  misc_feature (13)..(13): Xaa can be any naturally occurring amino acid
PVLDLFRELLNLXLEALKEKLK

SEQ ID NO: 103 - protein, 22 aa, Artificial sequence
  synthetic
PVLDEFRELLNEELEALKQKLK

SEQ ID NO: 104 - protein, 15 aa, Artificial sequence
  synthetic
PLLNELLEALKQKLK

SEQ ID NO: 105 - protein, 22 aa, Artificial sequence
  synthetic
PAADAFREAANEAAEAAKQKAK

SEQ ID NO: 106 - protein, 22 aa, Artificial sequence
  synthetic
PVLDLFREKLNEELEALKQKLK

SEQ ID NO: 107 - protein, 22 aa, Artificial sequence
  synthetic
KLKQKLAELLENLLERFLDLVP

SEQ ID NO: 108 - protein, 22 aa, Artificial sequence
  synthetic
  misc_feature: Xaa = Aib
  misc_feature (13)..(13): Xaa can be any naturally occurring amino acid
PVLDLFRWLLNEXLEALKQKLK

SEQ ID NO: 109 - protein, 22 aa, Artificial sequence
  synthetic
PVLDEFREKLNERLEALKQKLK

SEQ ID NO: 110 - protein, 22 aa, Artificial sequence
  synthetic
  misc_feature: Xaa = Aib
  misc_feature (13)..(14): Xaa can be any naturally occurring amino acid
PVLDEFREKLNEXXEALKQKLK

SEQ ID NO: 111 - protein, 22 aa, Artificial sequence
  synthetic
  misc_feature: Xaa = Aib
  misc_feature (13)..(13): Xaa can be any naturally occurring amino acid
PVLDEFREKLWEXWEALKQKLK

SEQ ID NO: 112 - protein, 22 aa, Artificial sequence
  synthetic
  misc_feature: Xaa = Aib
  misc_feature (13)..(13): Xaa can be any naturally occurring amino acid
PVLDEFREKLNEXSEALKQKLK

SEQ ID NO: 113 - protein, 22 aa, Artificial sequence
  synthetic
PVLDEFREKLNEPLEALKQKLK

SEQ ID NO: 114 - protein, 22 aa, Artificial sequence
  synthetic
  misc_feature: Xaa = Aib
  misc_feature (13)..(13): Xaa can be any naturally occurring amino acid
PVLDEFREKLNEXMEALKQKLK

SEQ ID NO: 115 - protein, 22 aa, Artificial sequence
  synthetic
  misc_feature: Xaa = Aib
  misc_feature (13)..(13): Xaa can be any naturally occurring amino acid
PKLDEFREKLNEXLEALKQKLK

SEQ ID NO: 116 - protein, 22 aa, Artificial sequence
  synthetic
  misc_feature: Xaa = Aib
  misc_feature (13)..(13): Xaa can be any naturally occurring amino acid
PHLDEFREKLNEXLEALKQKLK

SEQ ID NO: 117 - protein, 22 aa, Artificial sequence
  synthetic
  misc_feature: Xaa = Aib
  misc_feature (13)..(13): Xaa can be any naturally occurring amino acid
PELDEFREKLNEXLEALKQKLK

SEQ ID NO: 118 - protein, 22 aa, Artificial sequence
  synthetic
  misc_feature: Xaa = Aib
  misc_feature (13)..(13): Xaa can be any naturally occurring amino acid
PVLDEFREKLNEXLEALEQKLK

SEQ ID NO: 119 - protein, 22 aa, Artificial sequence
  synthetic
  misc_feature: Xaa = Aib
  misc_feature (17)..(17): Xaa can be any naturally occurring amino acid
PVLDEFREKLNEELEAXKQKLK

SEQ ID NO: 120 - protein, 22 aa, Artificial sequence
  synthetic
  misc_feature: Xaa = Aib
  misc_feature (16)..(16): Xaa can be any naturally occurring amino acid
PVLDEFREKLNEELEXLKQKLK

SEQ ID NO: 121 - protein, 22 aa, Artificial sequence
  synthetic
PVLDEFREKLNEELEALWQKLK

SEQ ID NO: 122 - protein, 22 aa, Artificial sequence
  synthetic
PVLDEFREKLNEELEWLKQKLK

SEQ ID NO: 123 - protein, 22 aa, Artificial sequence
  synthetic
QVLDLFRELLNELLEALKQKLK

SEQ ID NO: 124 - protein, 22 aa, Artificial sequence
  synthetic
  misc_feature: Xaa = Orn
  misc_feature (7)..(7): Xaa can be any naturally occurring amino acid
  misc_feature (18)..(18): Xaa can be any naturally occurring amino acid
  misc_feature (20)..(20): Xaa can be any naturally occurring amino acid
  misc_feature (22)..(22): Xaa can be any naturally occurring amino acid
PVLDLFXELLNELLEALXQXLX

SEQ ID NO: 125 - protein, 22 aa, Artificial sequence
  synthetic
NVLDLFRELLNELLEALKQKLK

SEQ ID NO: 126 - protein, 22 aa, Artificial sequence
  synthetic
PVLDLFRELLNELGEALKQKLK

SEQ ID NO: 127 - protein, 22 aa, Artificial sequence
  synthetic
PVLDLFRELLNELLELLKQKLK

SEQ ID NO: 128 - protein, 22 aa, Artificial sequence
  synthetic
PVLDLFRELLNELLEFLKQKLK

SEQ ID NO: 129 - protein, 22 aa, Artificial sequence
  synthetic
PVLELFNDLLRELLEALQKKLK

SEQ ID NO: 130 - protein, 22 aa, Artificial sequence
  synthetic
PVLELFNDLLRELLEALKQKLK

SEQ ID NO: 131 - protein, 22 aa, Artificial sequence
  synthetic
PVLELFKELLNELLDALRQKLK

SEQ ID NO: 132 - protein, 22 aa, Artificial sequence
  synthetic
PVLDLFRELLENLLEALQKKLK

SEQ ID NO: 133 - protein, 22 aa, Artificial sequence
  synthetic
PVLELFERLLEDLLQALNKKLK

SEQ ID NO: 134 - protein, 22 aa, Artificial sequence
  synthetic
  misc_feature: Xaa = Orn
  misc_feature (19)..(19): Xaa can be any naturally occurring amino acid
PVLELFERLLEDLLKALNXKLK

SEQ ID NO: 135 - protein, 22 aa, Artificial sequence
  synthetic
DVLDLFRELLNELLEALKQKLK

SEQ ID NO: 136 - protein, 22 aa, Artificial sequence
  synthetic
PALELFKDLLQELLEALKQKLK

SEQ ID NO: 137 - protein, 22 aa, Artificial sequence
  synthetic
PVLDLFRELLNEGLEAZKQKLK

SEQ ID NO: 138 - protein, 22 aa, Artificial sequence
  synthetic
PVLDLFRELLNEGLEWLKQKLK

SEQ ID NO: 139 - protein, 22 aa, Artificial sequence
  synthetic
PVLDLFRELWNEGLEALKQKLK

SEQ ID NO: 140 - protein, 22 aa, Artificial sequence
  synthetic
  misc_feature: Xaa = Orn
  misc_feature (18)..(18): Xaa can be any naturally occurring amino acid
  misc_feature (20)..(20): Xaa can be any naturally occurring amino acid
  misc_feature (22)..(22): Xaa can be any naturally occurring amino acid
PVLDLFRELLNEGLEALXQXLX

SEQ ID NO: 141 - protein, 22 aa, Artificial sequence
  synthetic
PVLDFFRELLNEGLEALKQKLK

SEQ ID NO: 142 - protein, 22 aa, Artificial sequence
  synthetic
PVLELFRELLNEGLEALKQKLK

SEQ ID NO: 143 - protein, 22 aa, Artificial sequence
  synthetic
PVLDLFRELLNEGLEALKQKLK

SEQ ID NO: 144 - protein, 22 aa, Artificial sequence
  synthetic
PVLELFENLLERLLDALQKKLK

SEQ ID NO: 145 - protein, 22 aa, Artificial sequence
  synthetic
GVLELFENLLERLLDALQKKLK

SEQ ID NO: 146 - protein, 22 aa, Artificial sequence
  synthetic
PVLELFENLLERLLDALQKKLK

SEQ ID NO: 147 - protein, 22 aa, Artificial sequence
  synthetic
PVLELFENLLERLFDALQKKLK

SEQ ID NO: 148 - protein, 22 aa, Artificial sequence
  synthetic
PVLELFENLLERLGDALQKKLK

SEQ ID NO: 149 - protein, 22 aa, Artificial sequence
  synthetic
PVLELFENLWERLLDALQKKLK

SEQ ID NO: 150 - protein, 22 aa, Artificial sequence
  synthetic
PLLELFENLLERLLDALQKKLK

SEQ ID NO: 151 - protein, 22 aa, Artificial sequence
  synthetic
PVLELFENLGERLLDALQKKLK

SEQ ID NO: 152 - protein, 22 aa, Artificial sequence
  synthetic
PVFELFENLLERLLDALQKKLK

SEQ ID NO: 153 - protein, 22 aa, Artificial sequence
  synthetic
AVLELFENLLERLLDALQKKLK

SEQ ID NO: 154 - protein, 22 aa, Artificial sequence
  synthetic
PVLELFENLLERGLDALQKKLK

SEQ ID NO: 155 - protein, 22 aa, Artificial sequence
  synthetic
PVLELFLNLWERLLDALQKKLK

SEQ ID NO: 156 - protein, 22 aa, Artificial sequence
  synthetic
PVLELFLNLLERLLDALQKKLK

SEQ ID NO: 157 - protein, 22 aa, Artificial sequence
  synthetic
PVLEFFENLLERLLDALQKKLK

SEQ ID NO: 158 - protein, 22 aa, Artificial sequence
  synthetic
PVLELFLNLLERLLDWLQKKLK

SEQ ID NO: 159 - protein, 22 aa, Artificial sequence
  synthetic
PVLDLFENLLERLLDALQKKLK

SEQ ID NO: 160 - protein, 22 aa, Artificial sequence
  synthetic
PVLELFENLLERLLDWLQKKLK

SEQ ID NO: 161 - protein, 22 aa, Artificial sequence
  synthetic
PVLELFENLLERLLEALQKKLK

SEQ ID NO: 162 - protein, 22 aa, Artificial sequence
  synthetic
PVLELFENWLERLLDALQKKLK

SEQ ID NO: 163 - protein, 22 aa, Artificial sequence
  synthetic
PVLELFENLLERLWDALQKKLK

SEQ ID NO: 164 - protein, 22 aa, Artificial sequence
  synthetic
PVLELFENLLERLLDAWQKKLK

SEQ ID NO: 165 - protein, 22 aa, Artificial sequence
  synthetic
PVLELFENLLERLLDLLQKKLK

SEQ ID NO: 166 - protein, 22 aa, Artificial sequence
  synthetic
PVLELFLNLLEKLLDALQKKLK

SEQ ID NO: 167 - protein, 22 aa, Artificial sequence
  synthetic
PVLELFENGLERLLDALQKKLK

SEQ ID NO: 168 - protein, 22 aa, Artificial sequence
  synthetic
PVLELFEQLLEKLLDALQKKLK

SEQ ID NO: 169 - protein, 22 aa, Artificial sequence
  synthetic
PVLELFENLLEKLLDALQKKLK

SEQ ID NO: 170 - protein, 22 aa, Artificial sequence
  synthetic
  misc_feature: Xaa = Orn
  misc_feature (12)..(12): Xaa can be any naturally occurring amino acid
  misc_feature (19)..(20): Xaa can be any naturally occurring amino acid
  misc_feature (22)..(22): Xaa can be any naturally occurring amino acid
PVLELFENLLEXLLDALQXXLX

SEQ ID NO: 171 - protein, 22 aa, Artificial sequence
  synthetic
PVLELFENLLEKLLDLLQKKLK

SEQ ID NO: 172 - protein, 22 aa, Artificial sequence
  synthetic
PVLELFLNLLERLGDALQKKLK

SEQ ID NO: 173 - protein, 22 aa, Artificial sequence
  synthetic
PVLDLFDNLLDRLLDLLNKKLK

SEQ ID NO: 174 - protein, 22 aa, Artificial sequence
  synthetic
PVLELFENLLERLLDALQKKLK

SEQ ID NO: 175 - protein, 22 aa, Artificial sequence
  synthetic
PVLELFENLLERLLELLNKKLK

SEQ ID NO: 176 - protein, 22 aa, Artificial sequence
  synthetic
PVLELWENLLERLLDALQKKLK

SEQ ID NO: 177 - protein, 22 aa, Artificial sequence
  synthetic
GVLELFLNLLERLLDALQKKLK

SEQ ID NO: 178 - protein, 22 aa, Artificial sequence
  synthetic
PVLELFDNLLEKLLEALQKKLR

SEQ ID NO: 179 - protein, 22 aa, Artificial sequence
  synthetic
PVLELFDNLLERLLDALQKKLK

SEQ ID NO: 180 - protein, 22 aa, Artificial sequence
  synthetic
PVLELFDNLLDKLLDALQKKLR

SEQ ID NO: 181 - protein, 22 aa, Artificial sequence
  synthetic
PVLELFENLLERWLDALQKKLK

SEQ ID NO: 182 - protein, 22 aa, Artificial sequence
  synthetic
PVLELFENLLEKLLEALQKKLK

SEQ ID NO: 183 - protein, 22 aa, Artificial sequence
  synthetic
PLLELFENLLEKLLDALQKKLK

SEQ ID NO: 184 - protein, 22 aa, Artificial sequence
  synthetic
PVLELFLNLLERLLDAWQKKLK

SEQ ID NO: 185 - protein, 22 aa, Artificial sequence
  synthetic
  misc_feature: Xaa = Orn
  misc_feature (19)..(20): Xaa can be any naturally occurring amino acid
  misc_feature (22)..(22): Xaa can be any naturally occurring amino acid
PVLELFENLLERLLDALQXXLX

SEQ ID NO: 186 - protein, 22 aa, Artificial sequence
  synthetic
PVLELFEQLLERLLDALQKKLK

SEQ ID NO: 187 - protein, 22 aa, Artificial sequence
  synthetic
PVLELFENLLERLLDALNKKLK

SEQ ID NO: 188 - protein, 22 aa, Artificial sequence
  synthetic
PVLELFENLLDRLLDALQKKLK

SEQ ID NO: 189 - protein, 22 aa, Artificial sequence
  synthetic
DVLELFENLLERLLDALQKKLK

SEQ ID NO: 190 - protein, 22 aa, Artificial sequence
  synthetic
PVLEFWDNLLDKLLDALQKKLR

SEQ ID NO: 191 - protein, 18 aa, Artificial sequence
  synthetic
PVLDLLRELLEELKQKLK

SEQ ID NO: 192 - protein, 18 aa, Artificial sequence
  synthetic
PVLDLFKELLEELKQKLK

SEQ ID NO: 193 - protein, 18 aa, Artificial sequence
  synthetic
PVLDLFRELLEELKQKLK

SEQ ID NO: 194 - protein, 18 aa, Artificial sequence
  synthetic
PVLELFRELLEELKQKLK

SEQ ID NO: 195 - protein, 18 aa, Artificial sequence
  synthetic
PVLELFKELLEELKQKLK

SEQ ID NO: 196 - protein, 18 aa, Artificial sequence
  synthetic
PVLDLFRELLEELKNKLK

SEQ ID NO: 197 - protein, 18 aa, Artificial sequence
  synthetic
PLLDLFRELLEELKQKLK

SEQ ID NO: 198 - protein, 18 aa, Artificial sequence
  synthetic
GVLDLFRELLEELKQKLK

SEQ ID NO: 199 - protein, 18 aa, Artificial sequence
  synthetic
PVLDLFRELWEELKQKLK

SEQ ID NO: 200 - protein, 18 aa, Artificial sequence
  synthetic
NVLDLFRELLEELKQKLK

SEQ ID NO: 201 - protein, 18 aa, Artificial sequence
  synthetic
PLLDLFKELLEELKQKLK

SEQ ID NO: 202 - protein, 18 aa, Artificial sequence
  synthetic
PALELFKDLLEELRQKLR

SEQ ID NO: 203 - protein, 18 aa, Artificial sequence
  synthetic
AVLDLFRELLEELKQKLK

SEQ ID NO: 204 - protein, 18 aa, Artificial sequence
  synthetic
PVLDFFRELLEELKQKLK

SEQ ID NO: 205 - protein, 18 aa, Artificial sequence
  synthetic
PVLDLFREWLEELKQKLK

SEQ ID NO: 206 - protein, 18 aa, Artificial sequence
  synthetic
PLLELLKELLEELKQKLK

SEQ ID NO: 207 - protein, 18 aa, Artificial sequence
  synthetic
PVLELLKELLEELKQKLK

SEQ ID NO: 208 - protein, 18 aa, Artificial sequence
  synthetic
PALELFKDLLEELRQRLK

SEQ ID NO: 209 - protein, 18 aa, Artificial sequence
  synthetic
PVLDLFRELLNELLQKLK

SEQ ID NO: 210 - protein, 18 aa, Artificial sequence
  synthetic
PVLDLFRELLEELKQKLK

SEQ ID NO: 211 - protein, 18 aa, Artificial sequence
  synthetic
  misc_feature: Xaa = Orn
  misc_feature (14)..(14): Xaa can be any naturally occurring amino acid
  misc_feature (16)..(16): Xaa can be any naturally occurring amino acid
  misc_feature (18)..(18): Xaa can be any naturally occurring amino acid
PVLDLFRELLEELXQXLX

SEQ ID NO: 212 - protein, 18 aa, Artificial sequence
  synthetic
  misc_feature: Xaa = Orn
  misc_feature (7)..(7): Xaa can be any naturally occurring amino acid
  misc_feature (14)..(14): Xaa can be any naturally occurring amino acid
  misc_feature (16)..(16): Xaa can be any naturally occurring amino acid
PVLDLFXELLEELXQXLK

SEQ ID NO: 213 - protein, 18 aa, Artificial sequence
  synthetic
PALELFKDLLEEFRQRLK

SEQ ID NO: 214 - protein, 18 aa, Artificial sequence
  synthetic
PVLDLFRELLEELKQKLK

SEQ ID NO: 215 - protein, 18 aa, Artificial sequence
  synthetic
PVLDLFRELLEEWKQKLK

SEQ ID NO: 216 - protein, 18 aa, Artificial sequence
  synthetic
PVLELFKELLEELKQKLK

SEQ ID NO: 217 - protein, 18 aa, Artificial sequence
  synthetic
PVLDLFRELLELLKQKLK

SEQ ID NO: 218 - protein, 18 aa, Artificial sequence
  synthetic
PVLDLFRELLNELLQKLK

SEQ ID NO: 219 - protein, 18 aa, Artificial sequence
  synthetic
PVLDLFRELLNELWQKLK

SEQ ID NO: 220 - protein, 18 aa, Artificial sequence
  synthetic
PVLDLFRELLEELQKKLK

SEQ ID NO: 221 - protein, 18 aa, Artificial sequence
  synthetic
DVLDLFRELLEELKQKLK

SEQ ID NO: 222 - protein, 18 aa, Artificial sequence
  synthetic
PVLDAFRELLEALLQLKK

SEQ ID NO: 223 - protein, 18 aa, Artificial sequence
  synthetic
PVLDAFRELLEALAQLKK

SEQ ID NO: 224 - protein, 18 aa, Artificial sequence
  synthetic
PVLDLFREGWEELKQKLK

SEQ ID NO: 225 - protein, 18 aa, Artificial sequence
  synthetic
PVLDAFRELAEALAQLKK

SEQ ID NO: 226 - protein, 18 aa, Artificial sequence
  synthetic
PVLDAFRELGEALLQLKK

SEQ ID NO: 227 - protein, 18 aa, Artificial sequence
  synthetic
PVLDLFRELGEELKQKLK

SEQ ID NO: 228 - protein, 18 aa, Artificial sequence
  synthetic
PVLDLFREGLEELKQKLK

SEQ ID NO: 229 - protein, 18 aa, Artificial sequence
  synthetic
PVLDLFRELLEEGKQKLK

SEQ ID NO: 230 - protein, 18 aa, Artificial sequence
  synthetic
PVLELFERLLEDLQKKLK

SEQ ID NO: 231 - protein, 18 aa, Artificial sequence
  synthetic
PVLDLFRELLEKLEQKLK

SEQ ID NO: 232 - protein, 18 aa, Artificial sequence
  synthetic
PLLELFKELLEELKQKLK

SEQ ID NO: 233 - protein, 18 aa, Artificial sequence
  synthetic
LDDLLQKWAEAFNQLLKK

SEQ ID NO: 234 - protein, 18 aa, Artificial sequence
  synthetic
EWLKAFYEKVLEKLKELF

SEQ ID NO: 235 - protein, 18 aa, Artificial sequence
  synthetic
EWLEAFYKKVLEKLKELF

SEQ ID NO: 236 - protein, 18 aa, Artificial sequence
  synthetic
DWLKAFYDKVAEKLKEAF

SEQ ID NO: 237 - protein, 18 aa, Artificial sequence
  synthetic
DWFKAFYDKVFEKFKEFF

SEQ ID NO: 238 - protein, 18 aa, Artificial sequence
  synthetic
GIKKFLGSIWKFIKAFVG

SEQ ID NO: 239 - protein, 18 aa, Artificial sequence
  synthetic
DWFKAFYDKVAEKFKEAF

SEQ ID NO: 240 - protein, 18 aa, Artificial sequence
  synthetic
DWLKAFYDKVAEKLKEAF

SEQ ID NO: 241 - protein, 18 aa, Artificial sequence
  synthetic
DWLKAFYDKVFEKFKEFF

SEQ ID NO: 242 - protein, 18 aa, Artificial sequence
  synthetic
EWLEAFYKKVLEKLKELP

SEQ ID NO: 243 - protein, 18 aa, Artificial sequence
  synthetic
DWFKAFYDKFFEKFKEFF

SEQ ID NO: 244 - protein, 18 aa, Artificial sequence
  synthetic
EWLKAFYEKVLEKLKELF

SEQ ID NO: 245 - protein, 18 aa, Artificial sequence
  synthetic
EWLKAEYEKVEEKLKELF

SEQ ID NO: 246 - protein, 18 aa, Artificial sequence
  synthetic
EWLKAEYEKVLEKLKELF

SEQ ID NO: 247 - protein, 18 aa, Artificial sequence
  synthetic
EWLKAFYKKVLEKLKELF

SEQ ID NO: 248 - protein, 15 aa, Artificial sequence
  synthetic
PVLDLFRELLEQKLK

SEQ ID NO: 249 - protein, 16 aa, Artificial sequence
  synthetic
PVLDLFRELLEELKQK

SEQ ID NO: 250 - protein, 16 aa, Artificial sequence
  synthetic
PVLDLFRELLEKLKQK

SEQ ID NO: 251 - protein, 15 aa, Artificial sequence
  synthetic
PVLDLFRELLEKLQK

SEQ ID NO: 252 - protein, 16 aa, Artificial sequence
  synthetic
PVLDLFRELLEALKQK

SEQ ID NO: 253 - protein, 16 aa, Artificial sequence
  synthetic
PVLDLFENLLERLKQK

SEQ ID NO: 254 - protein, 16 aa, Artificial sequence
  synthetic
PVLDLFRELLNELKQK

SEQ ID NO: 255 - protein, 18 aa, Artificial sequence
  synthetic
DWLKAFYDKVAEKLKEAF

SEQ ID NO: 256 - protein, 18 aa, Artificial sequence
  synthetic
DWLKAFYDKVAEKLKEAF

SEQ ID NO: 257 - protein, 18 aa, Artificial sequence
  synthetic
DWLKAFYDKVAEKLKEAF

SEQ ID NO: 258 - protein, 18 aa, Artificial sequence
  synthetic
DWFKAFYDKVAEKLKEAF

SEQ ID NO: 259 - protein, 18 aa, Artificial sequence
  synthetic
DWLKAFYDKVAEKFKEAF

SEQ ID NO: 260 - protein, 18 aa, Artificial sequence
  synthetic
DWFKAFYDKVAEKFKEAF

SEQ ID NO: 261 - protein, 18 aa, Artificial sequence
  synthetic
DWLKAFYDKVFEKFKEFF

SEQ ID NO: 262 - protein, 18 aa, Artificial sequence
  synthetic
DWLKAFYDKFFEKFKEFF

SEQ ID NO: 263 - protein, 18 aa, Artificial sequence
  synthetic
DWFKAFYDKFFEKFKEFF

SEQ ID NO: 264 - protein, 18 aa, Artificial sequence
  synthetic
DWLKAFYDKVAEKLKEFF

SEQ ID NO: 265 - protein, 18 aa, Artificial sequence
  synthetic
DWLKAFYDKVFEKFKEAF

SEQ ID NO: 266 - protein, 18 aa, Artificial sequence
  synthetic
DWLKAFYDKVFEKLKEFF

SEQ ID NO: 267 - protein, 18 aa, Artificial sequence
  synthetic
DWLKAFYDKVAEKFKEFF

SEQ ID NO: 268 - protein, 18 aa, Artificial sequence
  synthetic
DWLKAFYDKVFEKFKEFF

SEQ ID NO: 269 - protein, 18 aa, Artificial sequence
  synthetic
EWLKLFYEKVLEKFKEAF

SEQ ID NO: 270 - protein, 18 aa, Artificial sequence
  synthetic
EWLKAFYDKVAEKFKEAF

SEQ ID NO: 271 - protein, 18 aa, Artificial sequence
  synthetic
EWLKAFYDKVAEKLKEFF

SEQ ID NO: 272 - protein, 18 aa, Artificial sequence
  synthetic
EWLKAFYDKVFEKFKEAF

SEQ ID NO: 273 - protein, 18 aa, Artificial sequence
  synthetic
EWLKAFYDKVFEKLKEFF

SEQ ID NO: 274 - protein, 18 aa, Artificial sequence
  synthetic
EWLKAFYDKVAEKFKEFF

SEQ ID NO: 275 - protein, 18 aa, Artificial sequence
  synthetic
EWLKAFYDKVFEKFKEFF

SEQ ID NO: 276 - protein, 14 aa, Artificial sequence
  synthetic
AFYDKVAEKLKEAF

SEQ ID NO: 277 - protein, 14 aa, Artificial sequence
  synthetic
AFYDKVAEKFKEAF

SEQ ID NO: 278 - protein, 14 aa, Artificial sequence
  synthetic
AFYDKVAEKFKEAF

SEQ ID NO: 279 - protein, 14 aa, Artificial sequence
  synthetic
AFYDKFFEKFKEFF

SEQ ID NO: 280 - protein, 14 aa, Artificial sequence
  synthetic
AFYDKFFEKFKEFF

SEQ ID NO: 281 - protein, 14 aa, Artificial sequence
  synthetic
AFYDKVAEKFKEAF

SEQ ID NO: 282 - protein, 14 aa, Artificial sequence
  synthetic
AFYDKVAEKLKEFF

SEQ ID NO: 283 - protein, 14 aa, Artificial sequence
  synthetic
AFYDKVFEKFKEAF

SEQ ID NO: 284 - protein, 14 aa, Artificial sequence
  synthetic
AFYDKVFEKLKEFF

SEQ ID NO: 285 - protein, 14 aa, Artificial sequence
  synthetic
AFYDKVAEKFKEFF

SEQ ID NO: 286 - protein, 14 aa, Artificial sequence
  synthetic
KAFYDKVFEKFKEF

SEQ ID NO: 287 - protein, 14 aa, Artificial sequence
  synthetic
LFYEKVLEKFKEAF

SEQ ID NO: 288 - protein, 14 aa, Artificial sequence
  synthetic
AFYDKVAEKFKEAF

SEQ ID NO: 289 - protein, 14 aa, Artificial sequence
  synthetic
AFYDKVAEKLKEFF

SEQ ID NO: 290 - protein, 14 aa, Artificial sequence
  synthetic
AFYDKVFEKFKEAF

SEQ ID NO: 291 - protein, 14 aa, Artificial sequence
  synthetic
AFYDKVFEKLKEFF

SEQ ID NO: 292 - protein, 14 aa, Artificial sequence
  synthetic
AFYDKVAEKFKEFF

SEQ ID NO: 293 - protein, 14 aa, Artificial sequence
  synthetic
AFYDKVFEKFKEFF

SEQ ID NO: 294 - protein, 18 aa, Artificial sequence
  synthetic
DWLKALYDKVAEKLKEAL

SEQ ID NO: 295 - protein, 18 aa, Artificial sequence
  synthetic
DWFKAFYEKVAEKLKEFF

SEQ ID NO: 296 - protein, 18 aa, Artificial sequence
  synthetic
DWFKAFYEKFFEKFKEFF

SEQ ID NO: 297 - protein, 18 aa, Artificial sequence
  synthetic
EWLKALYEKVAEKLKEAL

SEQ ID NO: 298 - protein, 18 aa, Artificial sequence
  synthetic
EWLKAFYEKVAEKLKEAF

SEQ ID NO: 299 - protein, 18 aa, Artificial sequence
  synthetic
EWFKAFYEKVAEKLKEFF

SEQ ID NO: 300 - protein, 18 aa, Artificial sequence
  synthetic
EWLKAFYEKVFEKFKEFF

SEQ ID NO: 301 - protein, 18 aa, Artificial sequence
  synthetic
EWLKAFYEKFFEKFKEFF

SEQ ID NO: 302 - protein, 18 aa, Artificial sequence
  synthetic
EWFKAFYEKFFEKFKEFF

SEQ ID NO: 303 - protein, 18 aa, Artificial sequence
  synthetic
DFLKAWYDKVAEKLKEAW

SEQ ID NO: 304 - protein, 18 aa, Artificial sequence
  synthetic
EFLKAWYEKVAEKLKEAW

SEQ ID NO: 305 - protein, 18 aa, Artificial sequence
  synthetic
DFWKAWYDKVAEKLKEWW

SEQ ID NO: 306 - protein, 18 aa, Artificial sequence
  synthetic
EFWKAWYEKVAEKLKEWW

SEQ ID NO: 307 - protein, 18 aa, Artificial sequence
  synthetic
DKLKAFYDKVFEWAKEAF

SEQ ID NO: 308 - protein, 18 aa, Artificial sequence
  synthetic
DKWKAVYDKFAEAFKEFL

SEQ ID NO: 309 - protein, 18 aa, Artificial sequence
  synthetic
EKLKAFYEKVFEWAKEAF

SEQ ID NO: 310 - protein, 18 aa, Artificial sequence
  synthetic
EKWKAVYEKFAEAFKEFL

SEQ ID NO: 311 - protein, 18 aa, Artificial sequence
  synthetic
DWLKAFVDKFAEKFKEAY

SEQ ID NO: 312 - protein, 18 aa, Artificial sequence
  synthetic
EKWKAVYEKFAEAFKEFL

SEQ ID NO: 313 - protein, 18 aa, Artificial sequence
  synthetic
DWLKAFVYDKVFKLKEFF

SEQ ID NO: 314 - protein, 18 aa, Artificial sequence
  synthetic
EWLKAFVYEKVFKLKEFF

SEQ ID NO: 315 - protein, 18 aa, Artificial sequence
  synthetic
DWLRAFYDKVAEKLKEAF

SEQ ID NO: 316 - protein, 18 aa, Artificial sequence
  synthetic
EWLRAFYEKVAEKLKEAF

SEQ ID NO: 317 - protein, 18 aa, Artificial sequence
  synthetic
DWLKAFYDRVAEKLKEAF

SEQ ID NO: 318 - protein, 18 aa, Artificial sequence
  synthetic
EWLKAFYERVAEKLKEAF

SEQ ID NO: 319 - protein, 18 aa, Artificial sequence
  synthetic
DWLKAFYDKVAERLKEAF

SEQ ID NO: 320 - protein, 18 aa, Artificial sequence
  synthetic
EWLKAFYEKVAERLKEAF

SEQ ID NO: 321 - protein, 18 aa, Artificial sequence
  synthetic
DWLKAFYDKVAEKLREAF

SEQ ID NO: 322 - protein, 18 aa, Artificial sequence
  synthetic
EWLKAFYEKVAEKLREAF

SEQ ID NO: 323 - protein, 18 aa, Artificial sequence
  synthetic
DWLKAFYDRVAERLKEAF

SEQ ID NO: 324 - protein, 18 aa, Artificial sequence
  synthetic
EWLKAFYERVAERLKEAF

SEQ ID NO: 325 - protein, 18 aa, Artificial sequence
  synthetic
DWLRAFYDKVAEKLREAF

SEQ ID NO: 326 - protein, 18 aa, Artificial sequence
  synthetic
EWLRAFYEKVAEKLREAF

SEQ ID NO: 327 - protein, 18 aa, Artificial sequence
  synthetic
DWLRAFYDRVAEKLKEAF

SEQ ID NO: 328 - protein, 18 aa, Artificial sequence
  synthetic
EWLRAFYERVAEKLKEAF

SEQ ID NO: 329 - protein, 18 aa, Artificial sequence
  synthetic
DWLKAFYDKVAERLREAF

SEQ ID NO: 330 - protein, 18 aa, Artificial sequence
  synthetic
EWLKAFYEKVAERLREAF

SEQ ID NO: 331 - protein, 18 aa, Artificial sequence
  synthetic
DWLRAFYDKVAERLKEAF

SEQ ID NO: 332 - protein, 18 aa, Artificial sequence
  synthetic
EWLRAFYEKVAERLKEAF

SEQ ID NO: 333 - protein, 18 aa, Artificial sequence
  synthetic
FAEKFKEAVKDYFAKFWD

SEQ ID NO: 334 - protein, 18 aa, Artificial sequence
  synthetic
DWFKAFYDKVAEKFKEAF

SEQ ID NO: 335 - protein, 18 aa, Artificial sequence
  synthetic
DWLKAFYDKVAEKLKEAF

SEQ ID NO: 336 - protein, 34 aa, Artificial sequence
  synthetic
PALEDLRQGLLPVLESFKVFLSALEEYTKKLNTQ

SEQ ID NO: 337 - protein, 26 aa, Artificial sequence
  synthetic
WDRVKDLATVYVDVLKDSGRDYVSQF

SEQ ID NO: 338 - protein, 21 aa, Artificial sequence
  synthetic
  X (20)..(20): Xaa is Pyrrolysine
LKLLDNWDSVTSTFSKLREXL

SEQ ID NO: 339 - protein, 22 aa, Artificial sequence
  synthetic
  X (4)..(4): Xaa is Pyrrolysine
  X (19)..(19): Xaa is Pyrrolysine
PVTXEFWDNLEKETEGLRXEMS

SEQ ID NO: 340 - protein, 11 aa, Artificial sequence
  synthetic
KDLEEVKAKVQ

SEQ ID NO: 341 - protein, 11 aa, Artificial sequence
  synthetic
  X (11)..(11): Xaa is Pyrrolysine
KDLEEVKAKVX

SEQ ID NO: 342 - protein, 22 aa, Artificial sequence
  synthetic
PYLDDFQKKWQEEMELYRQKVE

SEQ ID NO: 343 - protein, 22 aa, Artificial sequence
  synthetic
  X (18)..(18): Xaa is Pyrrolysine
PLRAELQEGARQKLHELXEKLS

SEQ ID NO: 344 - protein, 22 aa, Artificial sequence
  synthetic
PLGEEMRDRARAHVDALRTHLA

SEQ ID NO: 345 - protein, 22 aa, Artificial sequence
  synthetic
PYSDELRQRLAARLEALKENGG

SEQ ID NO: 346 - protein, 22 aa, Artificial sequence
  synthetic
ARLAEYHAKATEHLSTLSEKAK

SEQ ID NO: 347 - protein, 11 aa, Artificial sequence
  synthetic
  X (8)..(8): Xaa is Pyrrolysine
PALEDLRXGLL

SEQ ID NO: 348 - protein, 22 aa, Artificial sequence
  synthetic
PVLESFKVSFLSALEEYTKKLN

SEQ ID NO: 349 - protein, 21 aa, Artificial sequence
  synthetic
PVLESFVSFLSALEEYTKKLN

SEQ ID NO: 350 - protein, 22 aa, Artificial sequence
  synthetic
PVLESFKVSFLSALEEYTKKLN

SEQ ID NO: 351 - protein, 24 aa, Artificial sequence
  synthetic
TVLLLTICSLEGALVRRQAKEPCV

SEQ ID NO: 352 - protein, 12 aa, Artificial sequence
  synthetic
QTVTDYGKDLME

SEQ ID NO: 353 - protein, 20 aa, Artificial sequence
  synthetic
  X (8)..(8): Xaa is Pyrrolysine
KVKSPELXAEAKSYFEKSKE

SEQ ID NO: 354 - protein, 24 aa, Artificial sequence
  synthetic
  X (20)..(20): Xaa is Pyrrolysine
VLTLALVAVAGARAEVSADXVATV

SEQ ID NO: 355 - protein, 22 aa, Artificial sequence
  synthetic
  X (11)..(11): Xaa is Pyrrolysine
  X (17)..(18): Xaa is Pyrrolysine
NNAKEAVEHLXKSELTXXLNAL

SEQ ID NO: 356 - protein, 25 aa, Artificial sequence
  synthetic
  X (18)..(18): Xaa is Pyrrolysine
LPVLVWLSIVLEGPAPAXGTPDVSS

SEQ ID NO: 357 - protein, 26 aa, Artificial sequence
  synthetic
LPVLVVVLSIVLEGPAPAQGTPDVSS

SEQ ID NO: 358 - protein, 21 aa, Artificial sequence
  synthetic
ALDKLKEFGNTLEDKARELIS

SEQ ID NO: 359 - protein, 22 aa, Artificial sequence
  synthetic
VVALLALLASARASEAEDASLL

SEQ ID NO: 360 - protein, 25 aa, Artificial sequence
  synthetic
  X (24)..(24): Xaa is Pyrrolysine
HLRKLRKRLLRDADDLQKRLAVYXA

SEQ ID NO: 361 - protein, 22 aa, Artificial sequence
  synthetic
AQAWGERLRARMEEMGSRTRDR

SEQ ID NO: 362 - protein, 20 aa, Artificial sequence
  synthetic
LDEVKEQVAEVRAKLEEQAQ

SEQ ID NO: 363 - protein, 18 aa, Artificial sequence
  synthetic
DWLKAFYDKVAEKLKEAF

SEQ ID NO: 364 - protein, 37 aa, Artificial sequence
  synthetic
DWLKAFYDKVAEKLKEAFPDWAKAAYDKAAEKAKEAA

SEQ ID NO: 365 - protein, 21 aa, Artificial sequence
  synthetic
PVLDLFRELLNELLEALKQKL

SEQ ID NO: 366 - protein, 22 aa, Artificial sequence
  synthetic
PVLDLFRELLNELLEALKQKLA

SEQ ID NO: 367 - protein, 22 aa, Artificial sequence
  synthetic
PVLDLFRELLNELLEALKQKLK

SEQ ID NO: 368 - protein, 22 aa, Artificial sequence
  synthetic
PVLDLFRELLNELLEALKQKLA

SEQ ID NO: 369 - protein, 22 aa, Artificial sequence
  synthetic
PVLDLFRELLNELLEALKKLLK

SEQ ID NO: 370 - protein, 22 aa, Artificial sequence
  synthetic
PVLDLFRELLNELLEALKKLLA

SEQ ID NO: 371 - protein, 22 aa, Artificial sequence
  synthetic
PLLDLFRELLNELLEALKKLLA